(19) United States
(12) Patent Application Publication
(10) Pub. No.: US 2012/0283217 A1
(43) Pub. Date: Nov. 8, 2012

(54) MODULATORS OF AMYLOID-BETA PRODUCTION

(75) Inventors: Mark A. Findeis, Belmont, MA (US); Steffen P. Creaser, Cambridge, MA (US)

(73) Assignee: SATORI PHARMACEUTICALS, INC., Cambridge, MA (US)

(21) Appl. No.: 13/550,125

(22) Filed: Jul. 16, 2012

Related U.S. Application Data
(63) Continuation of application No. 12/515,648, filed on Jul. 6, 2009, now abandoned.
(60) Provisional application No. 60/860,130, filed on Nov. 20, 2006.

(51) Int. Cl. C07J 53/00; C07J 51/00; A61K 31/695; A61K 31/566; C07J 17/00; A61K 31/58; C07J 9/00; C07J 21/00; C07J 7/00; A61K 31/57; C07J 41/00; C12N 5/071; A61P 25/28; A61P 25/16; A61P 25/00; A61P 3/10; A61P 27/12; A61P 21/00; A61P 43/00; A61K 31/575

(52) U.S. Cl. 514/63; 552/510; 514/182; 552/505; 514/180; 540/116; 514/174; 552/553; 514/178; 552/555; 540/31; 514/173; 552/606; 552/585; 514/181; 540/4; 540/114; 435/375

(57) ABSTRACT

As described herein, the present invention provides compounds useful for treating or lessening the severity of a neurodegenerative disorder. The present invention also provides methods of treating or lessening the severity of such disorders wherein said method comprises administering to a patient a compound of the present invention, or composition thereof. Said method is useful for treating or lessening the severity of, for example, Alzheimer's disease.

### CROSS-REFERENCE TO RELATED APPLICATIONS

This application claims priority to U.S. provisional application Ser. No. 60/860,130, filed Nov. 20, 2006, the entirety of which is hereby incorporated by reference.

### FIELD OF THE INVENTION

The present invention relates to compounds that modulate amyloid-beta peptide production, processes for their preparation, and uses thereof.

### BACKGROUND OF THE INVENTION

The central role of the long form of amyloid-beta peptide (A-beta), in particular Aβ(1-42), in Alzheimer's disease has been established through a variety of histopathological, genetic and biochemical studies. See Selkoe, D J, Physiol. Rev. 2001, 81:741-766, Alzheimer's disease: genes, proteins, and therapy, and Younkin S G, J Physiol Paris. 1998, 92:289-92, The role of Aβ(1-42) in Alzheimer's disease. Specifically, it has been found that deposition in the brain of Aβ(1-42) is an early and invariant feature of all forms of Alzheimer's disease. In fact, this occurs before a diagnosis of Alzheimer's disease is possible and before the deposition of the shorter primary form of A-beta, Aβ(1-40). See Parvathy S, et al. Arch Neural. 2001, 58:2025-32, Correlation between Abeta(x-40)-, Abeta(x-42)-, and Abeta(x-43)-containing amyloid plaques and cognitive decline. Further implication of Aβ(1-42) in disease etiology comes from the observation that mutations in presenilin (gamma secretase) genes associated with early onset familial forms of Alzheimer's disease uniformly result in increased levels of Aβ(1-42). See Ishii K, et al. Neurosci Lett. 1997, 228:17-20, Increased A beta(1-42(43))-plaque deposition in early-onset familial Alzheimer's disease brains with the deletion of exon 9 and the missense point mutation (H163R) in the PS-1 gene. Additional mutations in the amyloid precursor protein APP raise total Aβ and in some cases raise Aβ(1-42) alone. See Kosaka T, et al. Neurology, 48:741-5, The beta APP717 Alzheimer mutation increases the percentage of plasma amyloid-beta protein ending at A beta42(43). Although the various APP mutations may influence the type, quantity, and location of Aβ deposited, it has been found that the predominant and initial species deposited in the brain parenchyma is long Aβ(Mann). See Mann D M, et al. Am J Pathol. 1996, 148:1257-66, Predominant deposition of amyloid-beta 42(43) in plaques in cases of Alzheimer's disease and hereditary cerebral hemorrhage associated with mutations in the amyloid precursor protein gene.

In early deposits of Aβ, when most deposited protein is in the form of amorphous or diffuse plaques, virtually all of the Aβ is of the long form. See Gravina S A, et al. J Biol Chem, 270:7013-6, Amyloid beta protein (A beta) in Alzheimer's disease brain. Biochemical and immunocytochemical analysis with antibodies specific for forms ending at A beta 40 or A beta 42(43); Iwatsubo T, et al. Am J Pathol. 1996, 149:1823-30, Full-length amyloid-beta (1-42(43)) and amino-terminally modified and truncated amyloid-beta 42(43) deposit in diffuse plaques; and Roher A E, et al. Proc Natl Acad Sci USA. 1993, 90:10836-40, beta-Amyloid-(1-42) is a major component of cerebrovascular amyloid deposits: implications for the pathology of Alzheimer disease. These initial deposits of Aβ(1-42) then are able to seed the further deposition of both long and short forms of Aβ. See Tamaoka A, et al. Biochem Biophys Res Commun. 1994, 205:834-42, Biochemical evidence for the long-tail form (A beta 1-42/43) of amyloid beta protein as a seed molecule in cerebral deposits of Alzheimer's disease.

In transgenic animals expressing Aβ deposits were associated with elevated levels of Aβ(1-42), and the pattern of deposition is similar to that seen in human disease with Aβ(1-42) being deposited early followed by deposition of Aβ(1-40). See Rockenstein E, et al. J Neurosci Res. 2001, 66:573-82, Early formation of mature amyloid-beta protein deposits in a mutant APP transgenic model depends on levels of Abeta(1-42); and Terai K, et al. Neuroscience 2001, 104:299-310, beta-Amyloid deposits in transgenic mice expressing human beta-amyloid precursor protein have the same characteristics as those in Alzheimer's disease. Similar patterns and timing of deposition are seen in Down's Syndrome patients in which Aβ expression is elevated and deposition is accelerated. See Iwatsubo T, et al. Ann Neurol. 1995, 37:294-9, Amyloid beta protein (A beta) deposition: A beta 42(43) precedes A beta 40 in Down syndrome.

Accordingly, selective lowering of Aβ(1-42) thus emerges as a disease-specific strategy for reducing the amyloid forming potential of all forms of Aβ, slowing or stopping the formation of new deposits of Aβ, inhibiting the formation of soluble toxic oligomers of Aβ, and thereby slowing or halting the progression of neurodegeneration.

### SUMMARY OF THE INVENTION

As described herein, the present invention provides compounds useful as modulators of amyloid-beta production. Such compounds are useful for treating or lessening the severity of a neurodegenerative disorder. The present invention also provides methods of treating or lessening the severity of such disorders wherein said method comprises administering to a patient a compound of the present invention, or composition thereof. Said method is useful for treating or lessening the severity of, for example, Alzheimer's disease.

### DETAILED DESCRIPTION OF CERTAIN EMBODIMENTS OF THE INVENTION

### 1. General Description of Compounds of the Invention

According to one embodiment, the present invention provides a compound of formula I:

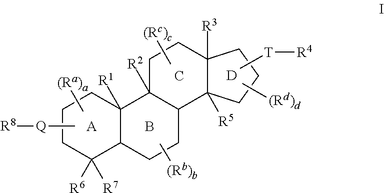

or a pharmaceutically acceptable salt thereof, wherein:
each of Ring A, Ring B, Ring C, and Ring D is independently saturated, partially unsaturated or aromatic;
R¹ and R² are each independently halogen, R, OR, a suitably protected hydroxyl group, SR, a suitably protected thiol group, N(R)₂, or a suitably protected amino group, or R¹ and R² are taken together with their intervening atoms to form a 3-7 membered saturated, partially unsaturated, or aryl ring having 0-2 heteroatoms independently selected from nitrogen, oxygen, or sulfur;
each R is independently hydrogen, an optionally substituted C₁₋₆ aliphatic group, or an optionally substituted 3-8 membered saturated, partially unsaturated, or aryl ring having 0-4 heteroatoms independently selected from nitrogen, oxygen, or sulfur, wherein:
   two R on the same nitrogen atom are optionally taken together with said nitrogen atom to form a 3-8 membered saturated, partially unsaturated, or aryl ring having 1-4 heteroatoms independently selected from nitrogen, oxygen, or sulfur;
R³ and R⁵ are each independently selected from halogen, R, OR, a suitably protected hydroxyl group, SR, a suitably protected thiol group, SO₂R, OSO₂R, N(R)₂, a suitably protected amino group, NR(CO)R, NR(CO)(CO)R, NR(CO)N(R)₂, NR(CO)OR, (CO)OR, O(CO)R, (CO)N(R)₂, or O(CO)N(R)₂, wherein R³ and R^{d} optionally form an epoxide or R⁵ and R^{d} optionally form an epoxide;
R⁶ and R⁷ are each independently selected from halogen, R, OR, SR, or N(R)₂, or R⁶ and R⁷ are taken together with their intervening atoms to form a 3-7 membered saturated, partially unsaturated, or aryl ring having 0-2 heteroatoms independently selected from nitrogen, oxygen, or sulfur;
T is independently a valence bond or an optionally substituted straight or branched, saturated or unsaturated, C₁₋₁₀ bivalent hydrocarbon chain wherein up to two methylene units of T are optionally and independently replaced by —O—, —N(R^{x})—, —S—, —C(O)—, —S(O)—, or —S(O)₂—, wherein two adjacent methylene units of T are optionally taken together with their intervening atoms to form an epoxide;
R⁴ is CN, C(R′)₃, C(R′)₂C(R″)₃, R, OR, a suitably protected hydroxyl group, SR, a suitably protected thiol group, SO₂R, OSO₂R, N(R)₂, a suitably protected amino group, NR(CO)R, NR(CO)(CO)R, NR(CO)N(R)₂, NR(CO)OR, (CO)OR, O(CO)R, (CO)N(R)₂, or O(CO)N(R)₂;
each of R′ and R″ is independently selected from R, OR, SR, SO₂R, OSO₂R, N(R)₂, NR(CO)R, NR(CO)(CO)R, NR(CO)N(R)₂, NR(CO)OR, (CO)OR, O(CO)R, (CO)N(R)₂, or O(CO)N(R)₂;
each of a, b, c, and d is 0-2;
each of R^{a}, R^{b}, and R^{c} is independently halogen, CN, R, OR, a suitably protected hydroxyl group, SR, a suitably protected thiol group, SO₂R, OSO₂R, N(R)₂, a suitably protected amino group, NR(CO)R, NR(CO)(CO)R, NR(CO)N(R)₂, NR(CO)OR, (CO)OR, O(CO)R, (CO)N(R)₂, O(CO)N(R)₂, ═O, ═S, or ═NH(R), wherein:
   two R^{a} groups, or an R^{a} group and R¹, on adjacent carbon atoms are optionally taken together with their intervening atoms to form an epoxide;
   two R^{b} groups, or an R^{b} group and R⁵, on adjacent carbon atoms are optionally taken together with their intervening atoms to form an epoxide; or
   two R^{c} groups, or an R^{c} group and either R² or R³, on adjacent carbon atoms are optionally taken together with their intervening atoms to form an epoxide;
each R^{d} is independently halogen, CN, R, OR, a suitably protected hydroxyl group, SR, a suitably protected thiol group, SO₂R, OSO₂R, N(R)₂, a suitably protected amino group, NR(CO)R, NR(CO)(CO)R, NR(CO)N(R)₂, NR(CO)OR, (CO)OR, O(CO)R, (CO)N(R)₂, O(CO)N(R)₂, ═O, ═S, or ═NH(R), wherein:
   two R^{d} groups on adjacent carbon atoms of Ring D are optionally taken together with their intervening atoms to form an epoxide;
   two R^{d} groups on the same carbon atom of Ring D are taken together to form an optionally substituted, straight or branched C₁₋₁₀ alkylidene group; or
   two R^{d} groups on the same carbon atom of Ring D are taken together to form an optionally substituted, saturated or unsaturated, 4-7 membered ring, having 0-2 heteroatoms independently selected from oxygen, nitrogen, or sulfur, wherein said ring formed thereby is Spiro fused to Ring D;
Q is a valence bond or an optionally substituted straight or branched, saturated or unsaturated, C₁₋₆ bivalent hydrocarbon chain wherein up to two methylene units of Q are optionally and independently replaced by —O—, —N(R)—, —S—, —C(O)—, —S(O)—, or —S(O)₂—; and
R⁸ is R, a suitably protected hydroxyl group, a suitably protected thiol group, a suitably protected amino group, an optionally substituted 3-8 membered saturated, partially unsaturated, or aryl monocyclic ring having 0-4 heteroatoms independently selected from nitrogen, oxygen, or sulfur, an optionally substituted 8-10 membered saturated, partially unsaturated, or aryl bicyclic ring having 0-4 heteroatoms independently selected from nitrogen, oxygen, or sulfur, a detectable moiety, a polymer residue, a peptide, or a sugar-containing or sugar-like moiety.

### 2. Definitions

Compounds of this invention include those described generally above, and are further illustrated by the embodiments, sub-embodiments, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry,” Thomas Sorrell, University Science Books, Sausalito: 1999, and “March's Advanced Organic Chemistry,” 5^{th} E a Ed.: Smith, M. B. and March, J., John Wiley & Sons, New York: 2001, the entire contents of which are hereby incorporated by reference.

As defined generally above, each of Ring A, Ring B, Ring C, and Ring D is independently saturated, partially unsaturated or aromatic. It will be appreciated that compounds of the present invention are contemplated as chemically feasible compounds. Accordingly, it will be understood by one of ordinary skill in the art that when any of Ring A, Ring B, Ring C, and Ring D is unsaturated, then certain substituents on that ring will be absent in order to satisfy general rules of valency. For example, if Ring C is unsaturated at the bond between Ring C and Ring D, then R³ and R⁵ will be absent. Alternatively, if Ring A is unsaturated at the bond between Ring A and Ring B, then R¹ will be absent. All combinations of saturation and unsaturation of any of Ring A, Ring B, Ring C, and Ring D are contemplated by the present invention. Thus, in order to satisfy general rules of valency, and depending on the degree of saturation or unsaturation of any of Ring A, Ring B, Ring C, and Ring D, the requisite presence or absence of each of R¹, R², R³, R⁴, R⁵, R⁶, R⁷, R⁸, R^{a}, R^{b}, R^{c}, R^{d}, -T-R⁴, and -Q-R⁸ is contemplated accordingly.

As described herein, compounds of the invention may optionally be substituted with one or more substituents, such as are illustrated generally above, or as exemplified by particular classes, subclasses, and species of the invention. It will be appreciated that the phrase “optionally substituted” is used interchangeably with the phrase “substituted or unsubstituted.” In general, the term “substituted,” whether preceded by the term “optionally” or not, refers to the replacement of hydrogen radicals in a given structure with the radical of a specified substituent. Unless otherwise indicated, an optionally substituted group may have a substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds.

The term “stable,” as used herein, refers to compounds that are not substantially altered when subjected to conditions to allow for their production, detection, and preferably their recovery, purification, and use for one or more of the purposes disclosed herein. In some embodiments, a stable compound or chemically feasible compound is one that is not substantially altered when kept at a temperature of 40° C. or less, in the absence of moisture or other chemically reactive conditions, for at least a week.

The term “aliphatic” or “aliphatic group,” as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1-10 aliphatic carbon atoms. In some embodiments, aliphatic groups contain 1-6 aliphatic carbon atoms. In yet other embodiments aliphatic groups contain 1-4 aliphatic carbon atoms. In some embodiments, “cycloaliphatic” (or “carbocycle” or “cycloalkyl”) refers to a monocyclic C₃-C₈ hydrocarbon or bicyclic C₈-C₁₂ hydrocarbon that is completely saturated or that contains one or more units of unsaturation, but which is not aromatic, that has a single point of attachment to the rest of the molecule wherein any individual ring in said bicyclic ring system has 3-7 members. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl. In other embodiments, an aliphatic group may have two geminal hydrogen atoms replaced with oxo (a bivalent carbonyl oxygen atom ═O), or a ring-forming substituent, such as —O-(straight or branched alkylene or alkylidene)-O— to form an acetal or ketal.

In certain embodiments, exemplary aliphatic groups include, but are not limited to, ethynyl, 2-propynyl, 1-propenyl, 2-butenyl, 1,3-butadienyl, 2-pentenyl, vinyl (ethenyl), allyl, isopropenyl, methyl, ethyl, propyl, isopropyl, cyclopropyl, butyl, isobutyl, sec-butyl, tert-butyl, pentyl, isopentyl, sec-pentyl, neo-pentyl, tert-pentyl, cyclopentyl, hexyl, isohexyl, sec-hexyl, cyclohexyl, 2-methylpentyl, tert-hexyl, 2,3-dimethylbutyl, 3,3-dimethylbutyl, 1,3-dimethylbutyl, and 2,3-dimethyl but-2-yl.

As used herein, the term “alkylidene” refers to a bivalent hydrocarbon group formed by mono or dialkyl substitution of a methylene. In certain embodiments, an alkylidene group has 1-10 carbon atoms. In other embodiments, an alkylidene group has 2-6, 1-5, 2-4, or 1-3 carbon atoms. Such groups include propylidene (═CHCH₂CH₃), ethylidene (═CHCH₃), and isopropylidene (═CH(CH₃)CH₃), and the like.

The term “heterocycle,” “heterocyclyl,” “heterocycloaliphatic,” or “heterocyclic” as used herein means non-aromatic, monocyclic, bicyclic, or tricyclic ring systems in which one or more ring members is an independently selected heteroatom. In some embodiments, the “heterocycle,” “heterocyclyl,” “heterocycloaliphatic,” or “heterocyclic” group has three to fourteen ring members in which one or more ring members is a heteroatom independently selected from oxygen, sulfur, nitrogen, or phosphorus, and each ring in the system contains 3 to 7 ring members.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl).

The term “unsaturated,” as used herein, means that a moiety has one or more units of unsaturation.

The term “aryl” used alone or as part of a larger moiety as in “aralkyl,” “aralkoxy,” or “aryloxyalkyl,” refers to monocyclic, bicyclic, and tricyclic ring systems having a total of five to fourteen ring members, wherein at least one ring in the system is aromatic and wherein each ring in the system contains 3 to 7 ring members. The term “aryl” may be used interchangeably with the term “aryl ring.” The term “aryl” also refers to heteroaryl ring systems as defined hereinbelow.

The term “heteroaryl,” used alone or as part of a larger moiety as in “heteroaralkyl” or “heteroarylalkoxy”, refers to monocyclic, bicyclic, and tricyclic ring systems having a total of five to fourteen ring members, wherein at least one ring in the system is aromatic, at least one ring in the system contains one or more heteroatoms, and wherein each ring in the system contains 3 to 7 ring members. The term “heteroaryl” may be used interchangeably with the term “heteroaryl ring” or the term “heteroaromatic.”

An aryl (including aralkyl, aralkoxy, aryloxyalkyl and the like) or heteroaryl (including hctcroaralkyl and heteroarylalkoxy and the like) group may contain one or more substituents. Suitable substituents on the unsaturated carbon atom of an aryl or heteroaryl group are selected from halogen; N₃, CN, R^{∘}; OR^{∘}; SR^{∘}; 1,2-methylene-dioxy; 1,2-ethylenedioxy; phenyl (Ph) optionally substituted with R^{∘}; —O(Ph) optionally substituted with R^{∘}; (CH₂)₁₋₂(Ph), optionally substituted with R^{∘}; CH═CH(Ph), optionally substituted with R^{∘}; NO₂; CN; N(R^{∘})₂; NR^{∘}C(O)R^{∘}; NR^{∘}C(O)N(R^{∘})₂; NR^{∘}CO₂R^{∘}; —NR^{∘}NR^{∘}C(O)R^{∘}; NR^{∘}NR^{∘}C(O)N(R^{∘})₂; NR^{∘}NR^{∘}CO₂R^{∘}; C(O)C(O)R^{∘}; C(O)CH₂C(O)R^{∘}; CO₂R^{∘}; C(O)R^{∘}; C(O)N(R^{∘})₂; OC(O)N(R^{∘})₂; S(O)₂R^{∘}; SO₂N(R^{∘})₂; S(O)R^{∘}; NR^{∘}SO₂N(R^{∘})₂; NR^{∘}SO₂R^{∘}; C(═S)N(R^{∘})₂; C(═NH)—N(R^{∘})₂; or (CH₂)₀₋₂NHC(O)R^{∘}wherein each independent occurrence of R^{∘}is selected from hydrogen, optionally substituted C₁₋₆ aliphatic, an unsubstituted 5-6 membered heteroaryl or heterocyclic ring, phenyl, O(Ph), or CH₂(Ph), or, notwithstanding the definition above, two independent occurrences of R^{∘}, on the same substituent or different substituents, taken together with the atom(s) to which each R^{∘}group is bound, form a 3-8 membered cycloalkyl, heterocyclyl, aryl, or heteroaryl ring having 0-4 heteroatoms independently selected from nitrogen, oxygen, or sulfur. Optional substituents on the aliphatic group of R^{∘}are selected from N₃, CN, NH₂, NH(C₁₋₄ aliphatic), N(C₁₋₄ aliphatic)₂, halogen, C₁₋₄ aliphatic, OH, O(C₁₋₄ aliphatic), NO₂, CN, CO₂H, CO₂(C₁₋₄ aliphatic), O(haloC₁₋₄ aliphatic), or haloC₁₋₄ aliphatic, wherein each of the foregoing C₁₋₄ aliphatic groups of R^{∘}is unsubstituted.

An aliphatic or heteroaliphatic group or a non-aromatic heterocyclic ring may contain one or more substituents. Suitable substituents on the saturated carbon of an aliphatic or heteroaliphatic group, or of a non-aromatic heterocyclic ring are selected from those listed above for the unsaturated carbon of an aryl or heteroaryl group and additionally include the following: ═O, ═S, ═PPh₃, ═NNHR*, ═NN(R*)₂, ═NNHC(O)R*, ═NNHCO₂(alkyl), ═NNHSO₂(alkyl), or ═NR*, where each R* is independently selected from hydrogen or an optionally substituted C₁₋₆ aliphatic. Optional substituents on the aliphatic group of R* are selected from NH₂, NH(C₁₋₄ aliphatic), N(C₁₋₄ aliphatic)₂, halogen, C₁₋₄ aliphatic, OH, O(C₁₋₄ aliphatic), NO₂, CN, CO₂H, CO₂(C₁₋₄ aliphatic), O(halo C₁₋₄ aliphatic), or halo(C₁₋₄ aliphatic), wherein each of the foregoing C₁₋₄ aliphatic groups of R* is unsubstituted.

Optional substituents on the nitrogen of a non-aromatic heterocyclic ring are selected from R⁺, N(R⁺)₂, C(O)R⁺, CO₂R⁺, C(O)C(O)R⁺, C(O)CH₂C(O)R⁺, SO₂R⁺, SO₂N(R⁺)₂, C(═S)N(R⁺)₂, C(═NH)—N(R⁺)₂, or NR⁺SO₂R⁺; wherein R⁺is hydrogen, an optionally substituted C₁₋₆ aliphatic, optionally substituted phenyl, optionally substituted O(Ph), optionally substituted CH₂(Ph), optionally substituted (CH₂)₁₋₂(Ph); optionally substituted CH═CH(Ph); or an unsubstituted 5-6 membered heteroaryl or heterocyclic ring having one to four heteroatoms independently selected from oxygen, nitrogen, or sulfur, or, notwithstanding the definition above, two independent occurrences of R⁺, on the same substituent or different substituents, taken together with the atom(s) to which each R⁺ group is bound, form a 3-8-membered cycloalkyl, heterocyclyl, aryl, or heteroaryl ring having 0-4 heteroatoms independently selected from nitrogen, oxygen, or sulfur. Optional substituents on the aliphatic group or the phenyl ring of R⁺ are selected from NH₂, NH(C₁₋₄ aliphatic), N(C₁₋₄ aliphatic)₂, halogen, C₁₋₄ aliphatic, OH, O(C₁₋₄ aliphatic), NO₂, CN, CO₂H, CO₂(C₁₋₄ aliphatic), O(halo C₁₋₄ aliphatic), or halo(C₁₋₄ aliphatic), wherein each of the foregoing C₁₋₄ aliphatic groups of R⁺ is unsubstituted.

As detailed above, in some embodiments, two independent occurrences of R^{∘}(or R⁺, or any other variable similarly defined herein), are taken together with the atom(s) to which each variable is bound to form a 3-8-membered cycloalkyl, heterocyclyl, aryl, or heteroaryl ring having 0-4 heteroatoms independently selected from nitrogen, oxygen, or sulfur. Exemplary rings that are formed when two independent occurrences of R^{∘}(or R⁺, or any other variable similarly defined herein) are taken together with the atom(s) to which each variable is bound include, but are not limited to the following: a) two independent occurrences of R^{∘}(or R⁺, or any other variable similarly defined herein) that are bound to the same atom and are taken together with that atom to form a ring, for example, N(R^{∘})₂, where both occurrences of R^{∘}are taken together with the nitrogen atom to form a piperidin-1-yl, piperazin-1-yl, or morpholin-4-yl group; and b) two independent occurrences of R^{∘}(or R⁺, or any other variable similarly defined herein) that are bound to different atoms and are taken together with both of those atoms to form a ring, for example where a phenyl group is substituted with two occurrences of OR^{∘}

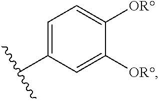

these two occurrences of R^{∘}are taken together with the oxygen atoms to which they are bound to form a fused 6-membered oxygen containing ring:

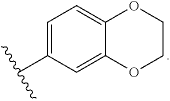

It will be appreciated that a variety of other rings can be formed when two independent occurrences of R^{∘}(or R¹, or any other variable similarly defined herein) are taken together with the atom(s) to which each variable is bound and that the examples detailed above are not intended to be limiting.

As used herein, the term “detectable moiety” is used interchangeably with the term “label” and relates to any moiety capable of being detected, e.g., primary labels and secondary labels. Primary labels, such as radioisotopes (e.g., ³²P, ³³P, ³⁵S, or ¹⁴C), or mass-tags, and fluorescent labels are signal generating reporter groups which can be detected without further modifications.

The term “secondary label” as used herein refers to moieties such as biotin and various protein antigens that require the presence of a second intermediate for production of a detectable signal. For biotin, the secondary intermediate may include streptavidin-enzyme conjugates. For antigen labels, secondary intermediates may include antibody-enzyme conjugates. Some fluorescent groups act as secondary labels because they transfer energy to another group in the process of nonradiative fluorescent resonance energy transfer (FRET), and the second group produces the detected signal.

The terms “fluorescent label,” “fluorescent dye,” and “fluorophore” as used herein refer to moieties that absorb light energy at a defined excitation wavelength and emit light energy at a different wavelength. Examples of fluorescent labels include, but are not limited to: Alexa Fluor dyes (Alexa Fluor 350, Alexa Fluor 488, Alexa Fluor 532, Alexa Fluor 546, Alexa Fluor 568, Alexa Fluor 594, Alexa Fluor 633, Alexa Fluor 660 and Alexa Fluor 680), AMCA, AMCA-S, BODIPY dyes (BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665), Carboxyrhodamine 6G, carboxy-X-rhodamine (ROX), Cascade Blue, Cascade Yellow, Coumarin 343, Cyanine dyes (Cy3, Cy5, Cy3.5, Cy5.5), Dansyl, Dapoxyl, Dialkylaminocoumarin, 4′,5′-Dichloro-2′,7′-dimethoxy-fluorescein, DM-NERF, Eosin, Erythrosin, Fluorescein, FAM, Hydroxycoumarin, IRDyes (IRD40, IRD 700, IRD 800), JOE, Lissamine rhodamine B, Marina Blue, Methoxycoumarin, Naphthofluorescein, Oregon Green 488, Oregon Green 500, Oregon Green 514, Pacific Blue, PyMPO, Pyrene, Rhodaminc B, Rhodaminc 6G, Rhodaminc Green, Rhodaminc Red, Rhodol Green, 2′,4′,5′,7′-Tetra-bromosulfone-fluorescein, Tetramethyl-rhodamine (TMR), Carboxytetramethylrhodamine (TAMRA), Texas Red, and Texas Red-X.

The term “mass-tag” as used herein refers to any moiety that is capable of being uniquely detected by virtue of its mass using mass spectrometry (MS) detection techniques. Examples of mass-tags include electrophore release tags such as N-[3-[4′-[(p-Methoxytetrafluorobenzyl)oxy]phenyl]-3-methylglyceronyl] isonipecotic Acid, 4′-[2,3,5,6-Tetrafluoro-4-(p entafluorophenoxyl)]methyl acetophenone, and their derivatives. The synthesis and utility of these mass-tags is described in U.S. Pat. Nos. 4,650,750, 4,709,016, 5,360,8191, 5,516,931, 5,602,273, 5,604,104, 5,610,020, and 5,650,270. Other examples of mass-tags include, but are not limited to, nucleotides, dideoxynucleotides, oligonucleotides of varying length and base composition, oligopeptides, oligosaccharides, and other synthetic polymers of varying length and monomer composition. A large variety of organic molecules, both neutral and charged (biomolecules or synthetic compounds) of an appropriate mass range (100-2000 Daltons) may also be used as mass-tags.

The term “substrate,” as used herein refers to any material or macromolecular complex to which a functionalized end-group of a compound of the present invention can be attached. Examples of commonly used substrates include, but are not limited to, glass surfaces, silica surfaces, plastic surfaces, metal surfaces, surfaces containing a metallic or chemical coating, membranes (e.g., nylon, polysulfone, silica), micro-beads (e.g., latex, polystyrene, or other polymer), porous polymer matrices (e.g., polyacrylamide gel, polysaccharide, polymethacrylate), and macromolecular complexes (e.g., protein, polysaccharide).

Unless otherwise stated, structures depicted herein are also meant to include all isomeric (e.g., enantiomeric, diastereomeric, and geometric (or conformational) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of the present compounds are within the scope of the invention.

Unless otherwise stated, all tautomeric forms of the compounds of the invention are within the scope of the invention.

Additionally, unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C- or ¹⁴C-enriched carbon are within the scope of this invention. Such compounds are useful, for example, as analytical tools or probes in biological assays.

### 3. Description of Exemplary Compounds

As defined generally above, R¹ and R² of formula I are each independently halogen, R, OR, a suitably protected hydroxyl group, SR, a suitably protected thiol group, N(R)₂, or a suitably protected amino group, or R¹ and R² are taken together to form a 3-7 membered saturated, partially unsaturated, or aryl ring having 0-2 heteroatoms independently selected from nitrogen, oxygen, or sulfur. In certain embodiments, R¹ and R² of formula I are each independently R or OR. In other embodiments, R¹ and R² of formula I are each independently R, wherein R is hydrogen or an optionally substituted C₁₋₆ aliphatic group. According to another aspect of the present invention, R¹ and R² of formula I are taken together to form a 3-6 membered saturated, partially unsaturated, or aryl ring having 0-2 heteroatoms independently selected from nitrogen, oxygen, or sulfur. Yet another aspect of the present invention provides a compound of formula I wherein R¹ and R² are taken together to form a 3-6 membered saturated carbocyclic ring. In other embodiments, R¹ and R² of formula I are taken together to form a cyclopropyl ring. Thus, according to yet another aspect of the present invention, a compound of formula I-a is provided:

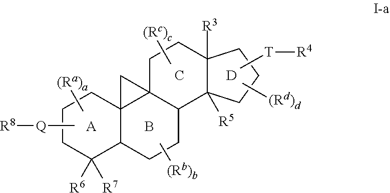

or a pharmaceutically acceptable salt thereof, wherein each variable is defined above and in classes and subclasses described above and herein for compounds of formula I.

In other embodiments, the present invention provides a compound of formula I-b, I-c, or I-d:

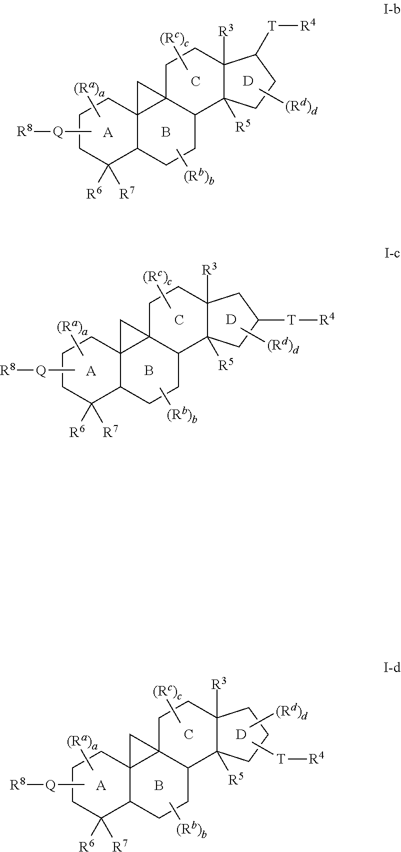

or a pharmaceutically acceptable salt thereof, wherein each variable is defined above and in classes and subclasses described above and herein for compounds of formula I.

In certain embodiments, the d variable of formula I is 0-1. In other embodiments, the d variable of formula I is 1. As defined generally above, the R^{d} group of formula I is each R^{d} is independently halogen, CN, R, OR, a suitably protected hydroxyl group, SR, a suitably protected thiol group, SO₂R, OSO₂R, N(R)₂, a suitably protected amino group, NR(CO)R, NR(CO)(CO)R, NR(CO)N(R)₂, NR(CO)OR, (CO)OR, O(CO)R, (CO)N(R)₂, O(CO)N(R)₂, ═O, ═S, or ═NH(R), wherein two R^{d} groups on adjacent carbon atoms of Ring D are optionally taken together with their intervening atoms to form an epoxide, or two R^{d} groups on the same carbon atom of Ring D are taken together to form an optionally substituted, straight or branched C₁₋₁₀ alkylidene group. In certain embodiments, R^{d} is R, OR, CN, ═O, or a suitably protected hydroxyl group. In other embodiments, R^{d} is hydrogen, OR, or ═O. In still other embodiments, R^{d} is OR or ═O.

In other embodiments, d is 2 and the two R^{d} groups are taken together with their intervening atoms to form an epoxide. Such compounds include those of formula I-e and I-f:

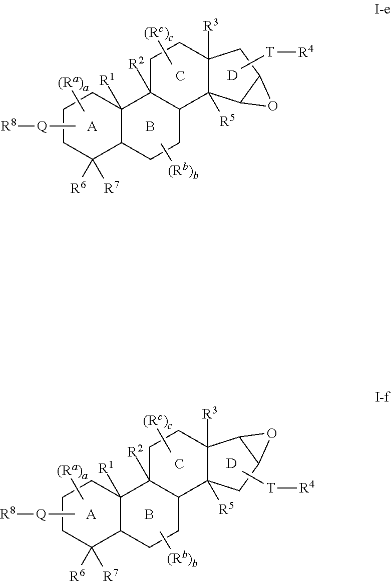

or a pharmaceutically acceptable salt thereof, wherein each variable is defined above and in classes and subclasses described above and herein.

In certain embodiments, d is 2 and two R^{d} groups on the same carbon atom are taken together to form an optionally substituted, straight or branched C₁₋₁₀ alkylidene group. Such alkylidene substituents on Ring D include those depicted in Table 1.

In other embodiments, d is 2 and two R^{d} groups on the same carbon atom of Ring D are taken together to form an optionally substituted, saturated or unsaturated, 4-7 membered ring, having 0-2 heteroatoms independently selected from oxygen, nitrogen, or sulfur, wherein said ring formed thereby is Spiro fused to Ring D. In certain embodiments, two R^{d} groups on the same carbon atom of Ring D are taken together to form an optionally substituted, saturated or unsaturated, 5-6 membered ring, having 0-1 heteroatoms independently selected from oxygen, nitrogen, or sulfur. In other embodiments, two R^{d} groups on the same carbon atom of Ring D are taken together to form an optionally substituted, 5 membered ring, having 1 oxygen atom. According to other aspects, the present invention provides a compound of formula I-g:

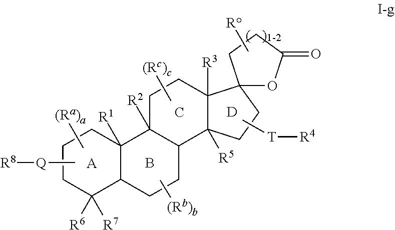

or a pharmaceutically acceptable salt thereof, wherein each variable is defined above and in classes and subclasses described above and herein.

As defined generally above, the T group of formula I is a valence bond or an optionally substituted straight or branched, saturated or unsaturated, C₁₋₁₀ bivalent hydrocarbon chain wherein up to two methylene units of T are optionally and independently replaced by —O—, —N(R^{x})—, —S—, —C(O)—, —S(O)—, or —S(O)₂—, wherein two adjacent methylene units of T are optionally taken together with their intervening atoms to form an epoxide. In certain embodiments, T is a valence bond or a straight or branched C₁₋₄ bivalent hydrocarbon chain wherein one methylene unit of T is optionally replaced by —O—, —N(R)—, or —S—. In some embodiments, T is a straight or branched C₁₋₄ bivalent hydrocarbon chain wherein one or two methylene units of T is replaced by —C(O)—, —O—, —N(R)—, or —S—. In other embodiments, T is a valence bond or a straight or branched C₁₋₄ bivalent hydrocarbon chain. In still other embodiments, T is a valence bond. In still other embodiments, T is a straight or branched C₁₋₄ bivalent hydrocarbon chain, wherein two adjacent methylene units of T are taken together to form an epoxide. Exemplary T groups of formula I include —CH(CH₃)CH₂CH₂C(═O)CH(CH₃)—, —CH(CH₃)CH₂CH₂CH═CH—, —CH(CH₃)CH₂CH₂—, ═CH—, —CH(CH₃)CH₂C(═O)—, and —CH(CH₃)CH═CH—.

As defined generally above, the R⁴ group of formula I is CN, C(R′)₃, C(R′)₂C(R″)₃, R, OR, a suitably protected hydroxyl group, SR, a suitably protected thiol group, SO₂R, OSO₂R, N(R)₂, a suitably protected amino group, NR(CO)R, NR(CO)(CO)R, NR(CO)N(R)₂, NR(CO)OR, (CO)OR, O(CO)R, (CO)N(R)₂, or O(CO)N(R)₂. In certain embodiments, R⁴ is R, halogen, OR, C(O)OR, or CN. In other embodiments, R⁴ is hydrogen. Exemplary R⁴ groups include, iodo, chloro, bromo, CN, OH, OMe, OEt, C(O)OMe, and C(O)OH.

When the R⁴ group of formula I is C(R′)₃ or C(R′)₂C(R″)₃, each R′ and R″ is independently selected from R, OR, SR, SO₂R, OSO₂R, N(R)₂, NR(CO)R, NR(CO)(CO)R, NR(CO)N(R)₂, NR(CO)OR, (CO)OR, O(CO)R, (CO)N(R)₂, or O(CO)N(R)₂. In certain embodiments, each R′ and R″ is independently R, OR, OC(O)R, SR, or N(R)₂. In other embodiments, each R′ and R″ is independently R, OR, or OC(O)R. Exemplary R′ and R″ groups include hydrogen, CH₃, OH, and OC(O)CH₃.

As defined generally above, the R⁴ group of formula I is, inter alia, a suitably protected hydroxyl group, a suitably protected thiol group, or a suitably protected amino group. Hydroxyl protecting groups are well known in the art and include those described in detail in Protecting Groups in Organic Synthesis, T. W. Greene and P. G. M. Wuts, 3^{Id} edition, John Wiley & Sons, 1999, the entirety of which is incorporated herein by reference. Examples of suitably protected hydroxyl groups of the R⁴ group of formula I further include, but are not limited to, esters, allyl ethers, ethers, silyl ethers, alkyl ethers, arylalkyl ethers, and alkoxyalkyl ethers. Examples of such esters include formates, acetates, carbonates, and sulfonates. Specific examples include formate, benzoyl formate, chloroacetate, trifluoroacetate, methoxyacetate, triphenylmethoxyacetate, p-chlorophenoxyacetate, 3-phenylpropionate, 4-oxopentanoate, 4,4-(ethylenedithio)pentanoate, pivaloate (trimethylacetyl), crotonate, 4-methoxy-crotonate, benzoate, p-benzylbenzoate, 2,4,6-trimethylbenzoate, carbonates such as methyl, 9-fluorenylmethyl, ethyl, 2,2,2-trichloroethyl, 2-(trimethylsilyl)ethyl, 2-(phenylsulfonyl)ethyl, vinyl, allyl, and p-nitrobenzyl. Examples of such silyl ethers include trimethylsilyl, triethylsilyl, t-butyldimethylsilyl, t-butyldiphenylsilyl, triisopropylsilyl, and other trialkylsilyl ethers. Alkyl ethers include methyl, benzyl, p-methoxybenzyl, 3,4-dimethoxybenzyl, trityl, t-butyl, allyl, and allyloxycarbonyl ethers or derivatives. Alkoxyalkyl ethers include acetals such as methoxymethyl, methylthiomethyl, (2-methoxyethoxy)methyl, benzyloxymethyl, beta-(trimethylsilyl)ethoxymethyl, and tetrahydropyranyl ethers. Examples of arylalkyl ethers include benzyl, p-methoxybenzyl (MPM), 3,4-dimethoxybenzyl, O-nitrobenzyl, p-nitrobenzyl, p-halobenzyl, 2,6-dichlorobenzyl, p-cyanobenzyl, 2- and 4-picolyl.

Thiol protecting groups are well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Suitably protected thiol groups of the R⁴ moiety of formula I include, but are not limited to, disulfides, thioethers, silyl thioethers, thioesters, thiocarbonates, thiocarbamates, and the like. Examples of such groups include, but are not limited to, alkyl thioethers, benzyl and substituted benzyl thioethers, triphenylmethyl thioethers, and trichlorocthoxycarbonyl, to name but a few.

According to another aspect of the present invention, the R⁴ moiety of formula I is a thiol protecting group that is removable under neutral conditions e.g. with AgNO₃, HgCl₂, and the like. Other neutral conditions include reduction using a suitable reducing agent. Suitable reducing agents include dithiothreitol (DTT), mercaptoethanol, dithionite, reduced glutathione, reduced glutaredoxin, reduced thioredoxin, substituted phosphines such as tris carboxyethyl phosphine (TCEP), and any other peptide or organic based reducing agent, or other reagents known to those of ordinary skill in the art. According to yet another aspect of the present invention, the R⁴ moiety of formula I is a thiol protecting group that is “photocleavable”. Such suitable thiol protecting groups are known in the art and include, but are not limited to, a nitrobenzyl group, a tetrahydropyranyl (THP) group, a trityl group, —CH₂SCH₃ (MTM), dimethylmethoxymethyl, or —CH₂—S—S-pyridin-2-yl. One of ordinary skill in the art would recognize that many of the suitable hydroxyl protecting groups, as described herein, are also suitable as thiol protecting groups.

In certain embodiments, the R⁴ group of formula I is a suitably protected amino group. Amino protecting groups are well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Suitably protected amino groups of said R⁴ moiety further include, but are not limited to, aralkylamines, carbamates, cyclic imides, allyl amines, amides, and the like. Examples of such groups include t-butyloxycarbonyl (BOC), ethyloxycarbonyl, methyloxycarbonyl, trichloroethyloxycarbonyl, allyloxycarbonyl (Alloc), benzyloxocarbonyl (CBZ), allyl, phthalimide, benzyl (Bn), fluorenylmethylcarbonyl (Fmoc), formyl, acetyl, chloroacetyl, dichloroacetyl, trichloroacetyl, phenylacetyl, trifluoroacetyl, benzoyl, and the like. In certain embodiments, the amino protecting group of the R⁴ moiety is phthalimido. In still other embodiments, the amino protecting group of the R⁴ moiety is a tert-butyloxycarbonyl (BOC) group.

As defined generally above, the R³ and R⁵ groups of formula I are each independently selected from halogen, R, OR, a suitably protected hydroxyl group, SR, a suitably protected thiol group, SO₂R, OSO₂R, N(R)₂, a suitably protected amino group, NR(CO)R, NR(CO)(CO)R, NR(CO)N(R)₂, NR(CO)OR, (CO)OR, O(CO)R, (CO)N(R)₂, or O(CO)N(R)₂, wherein R³ and R^{d} optionally form an epoxide or R⁵ and R^{d} optionally form an epoxide. In certain embodiments, R³ and R⁵ are each independently R, OR, or a suitably protected hydroxyl group. In other embodiments, only one of R³ and R⁵ is hydrogen. In still other embodiments, neither of R³ and R⁵ is hydrogen. According to other aspects, either R³ and R^{d} form an epoxide or R⁵ and R^{d} form an epoxide. According to other embodiments, R³ and R^{c}, or R⁵ and R^{b}, are taken together with their intervening atoms to form an epoxide.

As defined generally above, b is 0-2 and R^{b} is halogen, CN, R, OR, a suitably protected hydroxyl group, SR, a suitably protected thiol group, SO₂R, OSO₂R, N(R)₂, a suitably protected amino group, NR(CO)R, NR(CO)(CO)R, NR(CO)N(R)₂, NR(CO)OR, (CO)OR, O(CO)R, (CO)N(R)₂, O(CO)N(R)₂, ═O, ═S, or ═NH(R), or two R^{b} groups on adjacent carbon atoms are optionally taken together with their intervening atoms to form an epoxide. In certain embodiments, b is 1. In other embodiments, the present invention provides a compound wherein b is 1 and said compound is of formula I-h:

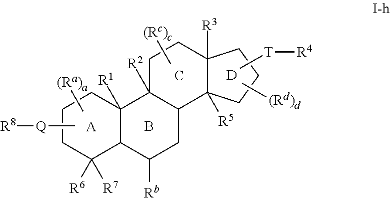

or a pharmaceutically acceptable salt thereof, wherein each variable is defined above and in classes and subclasses described above and herein.

In certain embodiments, the R^{b} group of formula I-h is other than hydrogen. In other embodiments, the R^{b} group of formula I-h is ═O, R, OR, or a suitably protected hydroxyl group.

As defined generally above, the Q group of formula I is a valence bond or an optionally substituted straight or branched, saturated or unsaturated, C₁₋₆ bivalent hydrocarbon chain wherein up to two methylene units of Q are optionally and independently replaced by —O—, —N(R)—, —S—, —S(O)—, or —S(O)₂—. In certain embodiments, Q is a an optionally substituted straight or branched, saturated or unsaturated, C₁₋₂ bivalent hydrocarbon chain wherein up to one methylene unit of Q is optionally replaced by —O—, —N(R)—, or —S—. In other embodiments, Q is —O—. In still other embodiments, Q is —N(R)— or (S).

As defined generally above, the R⁸ group of formula I is R, a suitably protected hydroxyl group, a suitably protected thiol group, a suitably protected amino group, an optionally substituted 3-8 membered saturated, partially unsaturated, or aryl monocyclic ring having 0-4 heteroatoms independently selected from nitrogen, oxygen, or sulfur, an optionally substituted 8-10 membered saturated, partially unsaturated, or aryl bicyclic ring having 0-4 heteroatoms independently selected from nitrogen, oxygen, or sulfur, a detectable moiety, a polymer residue, a peptide, or a sugar-containing or sugar-like moiety.

In certain embodiments, the R⁸ group of formula I is a sugar-containing group. Such sugar-containing groups are well known to one of ordinary skill in the art and include those described in detail in “Essentials of Glycobiology” Edited by Varki, A., et al., Cold Spring Harbor Laboratory Press. Cold Spring Harbor, N.Y. 2002. In certain embodiments, the R⁸ group of formula I is a glycoside. Exemplary R⁸ groups include arabinopyranosides and xylopyranosides. In certain embodiments, the R⁸ group of formula I is a xylopyranoside. In certain embodiments, the R⁸ group of formula I is an arabinopyranoside. In still other embodiments, the R⁸ group of formula I is

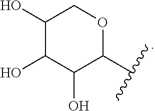

According to another embodiment, the R⁸ group of formula I is

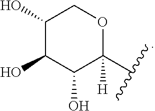

Yet another embodiment provides a compound of formula I wherein R⁸ is

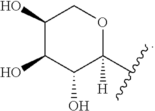

According to another aspect of the present invention, the R⁸ group of formula I is a sugar-mimetic. Such sugar-mimetics are well known to one of ordinary skill in the art and include those described in detail in “Essentials of Glycobiology.” For example, sugar-mimetic groups contemplated by the present invention include cyclitols and the like. In certain embodiments, R⁸ is a cyclitol moiety, wherein said cyclitol is a cycloalkane containing one hydroxyl group on each of three or more ring atoms, as defined by IUPAC convention. In other embodiments, such cyclitol moieties include inositols such as scyllo-inositol.

In addition, suitable sugar-like moieties of the R⁸ group of formula I include acyclic sugar groups. Such groups include linear alkylols and erythritols, to name but a few. It will be appreciated that sugar groups can exist in either cyclic or acyclic form. Accordingly, acyclic forms of a sugar group are contemplated by the present invention as a suitable sugar-like moiety of the R⁸ group of formula I.

In certain embodiments, the R⁸ group of formula I is a detectable moiety. In other embodiments, the R⁸ group of formula I is a fluorescent label, fluorescent dye, or fluorophorc as defined herein, supra.

According to another aspect of the present invention, the R⁸ group of formula I is a polymer residue. Polymer residues are well known in the art and include those described in detail in “Chemistry of Protein Conjugation and Cross-Linking” Shan S. Wong, CRC Press. Boca Raton, Fla. 1991. Suitable polymer residues of the R⁸ group of formula I include poly(alkylene oxides), such as PEG, poly(amino acids), and other polymer residues capable of conjugation to a compound of the present invention.

As defined generally above, the R⁸ group of formula I is, inter cilia, a suitably protected hydroxyl group, a suitably protected thiol group, or a suitably protected amino group. Hydroxyl protecting groups are well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Examples of suitable hydroxyl protecting groups of the R⁸ group of formula I further include, but are not limited to, esters, allyl ethers, ethers, silyl ethers, alkyl ethers, arylalkyl ethers, and alkoxyalkyl ethers. Examples of such esters include formates, acetates, carbonates, and sulfonates. Specific examples include formate, benzoyl formate, chloroacetate, trifluoroacetate, methoxyacetate, triphenylmethoxyacetate, p-chlorophenoxyacetate, 3-phenylpropionate, 4-oxopentanoate, 4,4-(ethylenedithio)pentanoate, pivaloate (trimethylacetyl), crotonate, 4-methoxy-crotonate, benzoate, p-benzylbenzoate, 2,4,6-trimethylbenzoate, carbonates such as methyl, 9-fluorenylmethyl, ethyl, 2,2,2-trichloroethyl, 2-(trimethylsilyl)ethyl, 2-(phenylsulfonyl)ethyl, vinyl, allyl, and p-nitrobenzyl. Examples of such silyl ethers include trimethylsilyl, triethylsilyl, t-butyldimethylsilyl, t-butyldiphenylsilyl, triisopropylsilyl, and other trialkylsilyl ethers. Alkyl ethers include methyl, benzyl, p-methoxybenzyl, 3,4-dimethoxybenzyl, trityl, t-butyl, allyl, and allyloxycarbonyl ethers or derivatives. Alkoxyalkyl ethers include acetals such as methoxymethyl, methylthiomethyl, (2-methoxyethoxy)methyl, benzyloxymethyl, beta-(trimethylsilyl)ethoxymethyl, and tetrahydropyranyl ethers. Examples of aryl alkyl ethers include benzyl, p-methoxybenzyl (MPM), 3,4-dimethoxybenzyl, O-nitrobenzyl, p-nitrobenzyl, p-halobenzyl, 2,6-dichlorobenzyl, p-cyanobenzyl, 2- and 4-picolyl. In certain embodiments, R⁸ is acetyl. In other embodiments, R⁸ forms a silyl ether with the oxygen atom to which it is attached.

Thiol protecting groups are well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Suitable thiol protecting groups of the R⁸ moiety of formula I include, but are not limited to, disulfides, thioethers, silyl thioethers, thioesters, thiocarbonates, thiocarbamates, and the like. Examples of such groups include, but are not limited to, alkyl thioethers, benzyl and substituted benzyl thioethers, triphenylmethyl thioethers, trichloroethoxycarbonyl, to name but a few.

According to another aspect of the present invention, the R⁸ moiety of formula I is a thiol protecting group that is removable under neutral conditions e.g. with AgNO₃, HgCl₂, and the like. Other neutral conditions include reduction using a suitable reducing agent. Suitable reducing agents include dithiothreitol (DTT), mercaptoethanol, dithionite, reduced glutathione, reduced glutaredoxin, reduced thioredoxin, substituted phosphines such as tris carboxyethyl phosphine (TCEP), and any other peptide or organic based reducing agent, or other reagents known to those of ordinary skill in the art. According to yet another aspect of the present invention, the R⁸ moiety of formula I is a thiol protecting group that is “photocleavable.” Such suitable thiol protecting groups are known in the art and include, but are not limited to, a nitrobenzyl group, a tetrahydropyranyl (THP) group, a trityl group, —CH₂SCH₃ (MTM), dimethylmethoxymethyl, or —CH₂—S—S-pyridin-2-yl. One of ordinary skill in the art would recognize that many of the suitable hydroxyl protecting groups, as described herein, are also suitable as thiol protecting groups.

In certain embodiments, the R⁸ group of formula I is a suitably protected amino group. Amino protecting groups are well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Suitable amino protecting groups of said R⁸ moiety further include, but are not limited to, aralkylamines, carbamates, cyclic imides, allyl amines, amides, and the like. Examples of such groups include t-butyloxycarbonyl (BOC), ethyloxycarbonyl, methyloxycarbonyl, trichloroethyloxycarbonyl, allyloxycarbonyl (Alloc), benzyloxocarbonyl (CBZ), allyl, phthalimide, benzyl (Bn), fluorenylmethylcarbonyl (Fmoc), formyl, acetyl, chloroacetyl, dichloroacetyl, trichloroacetyl, phenylacetyl, trifluoroacetyl, benzoyl, and the like. In certain embodiments, the amino protecting group of the R⁸ moiety is phthalimido. In still other embodiments, the amino protecting group of the R⁸ moiety is a tert-butyloxycarbonyl (BOC) group.

As described generally above, the present invention provides a compound of formula I:

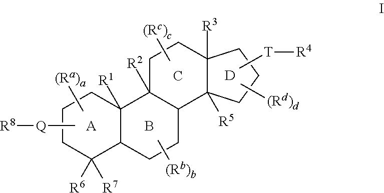

or a pharmaceutically acceptable salt thereof, wherein each variable is defined above and in classes and subclasses described above and herein. In certain embodiments, the present invention provides a compound of formula I having the stereochemistry as depicted in formula II:

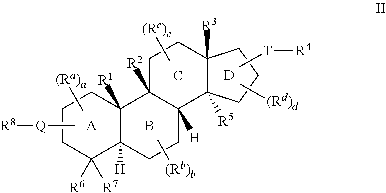

or a pharmaceutically acceptable salt thereof, wherein each variable is defined above and in classes and subclasses described above and herein for compounds of formula I.

Unless otherwise specified, the stereochemistry of compounds of formula I contemplated by the invention is as depicted in formula II.

As defined generally above, each of Ring A, Ring B, Ring C, and Ring D is independently saturated, partially unsaturated or aromatic. In certain embodiments, Ring A is aromatic and therefore R¹ and R⁶ are absent, thus forming a compound of formula III:

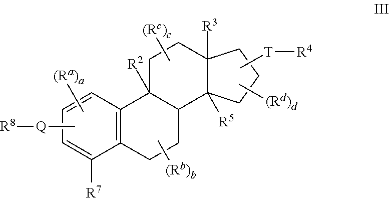

or a pharmaceutically acceptable salt thereof, wherein each variable is defined above and in classes and subclasses described above and herein for compounds of formula I.

In other embodiments, the present invention provides a compound of formula III-a:

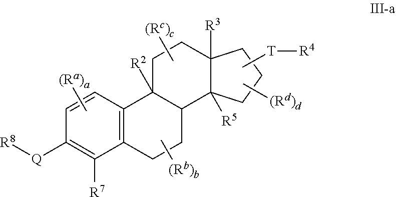

or a pharmaceutically acceptable salt thereof, wherein each variable is defined above and in classes and subclasses described above and herein for compounds of formula I.

According to another embodiment, Ring B is unsaturated and the present invention provides a compound of formula IV:

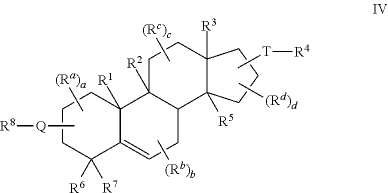

or a pharmaceutically acceptable salt thereof, wherein each variable is defined above and in classes and subclasses described above and herein for compounds of formula I.

In other embodiments, the present invention provides a compound of formula IV-a:

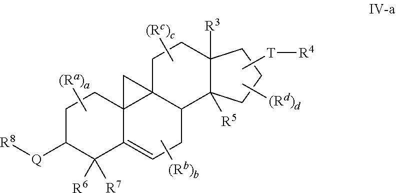

or a pharmaceutically acceptable salt thereof, wherein each variable is defined above and in classes and subclasses described above and herein for compounds of formula I.

In certain embodiments, the present invention provides a compound of formula V-a, V-b, V-c, or V-d:

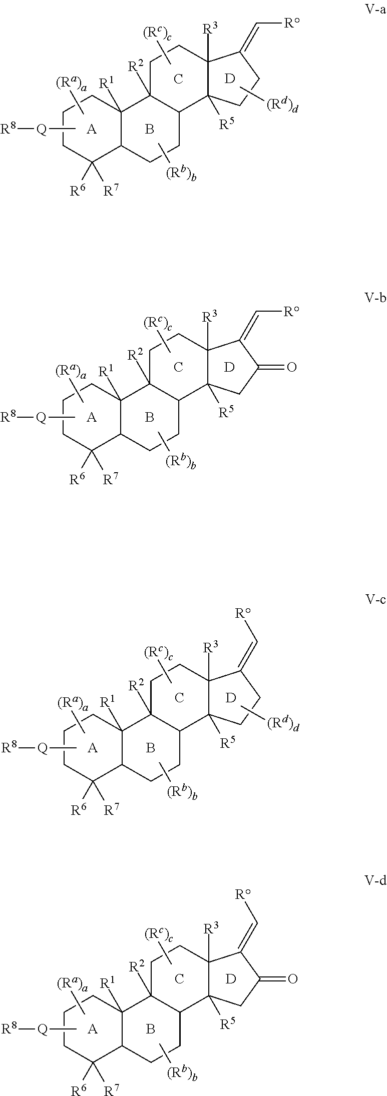

or a pharmaceutically acceptable salt thereof, wherein each variable is defined above and in classes and subclasses described above and herein for compounds of formula I.

In other embodiments, the present invention provides a compound of formula VI-a or VI-b:

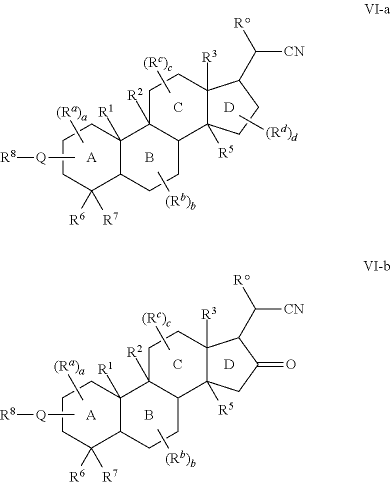

or a pharmaceutically acceptable salt thereof, wherein each variable is defined above and in classes and subclasses described above and herein for compounds of formula I.

Exemplary compounds of the present invention are set forth in Table 1, below. Additional exemplary compounds of the present invention are set forth in the Examples section, infra.

TABLE 1
Exemplary Compounds of Formula I

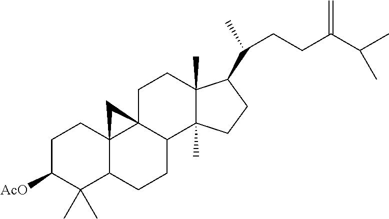

I-1

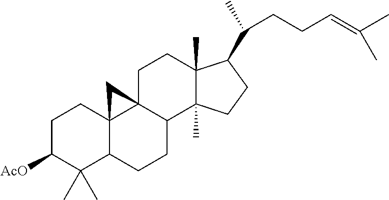

I-2

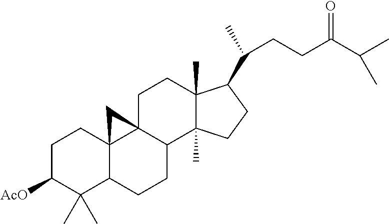

I-3

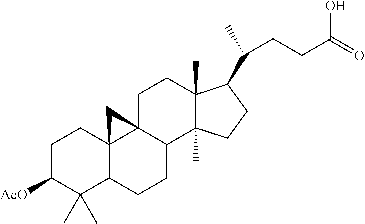

I-4

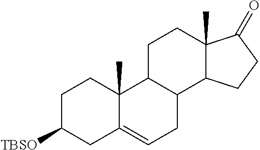

I-5

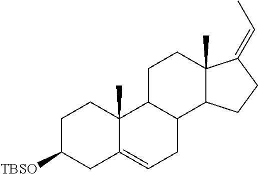

I-6

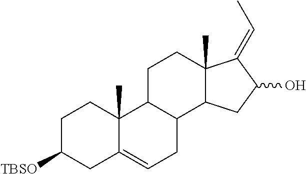

I-7

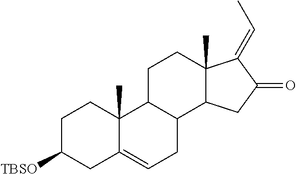

I-8

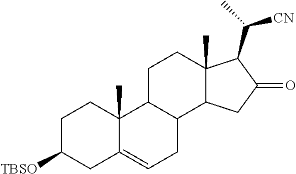

I-9

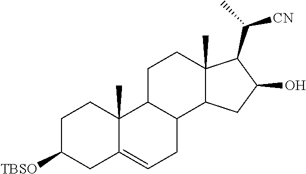

I-10

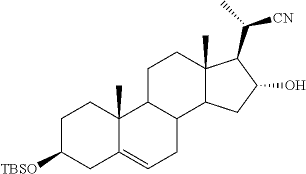

I-11

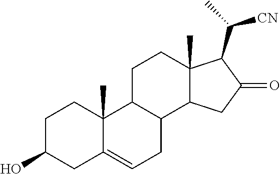

I-12

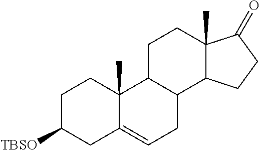

I-13

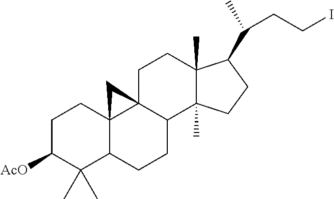

I-14

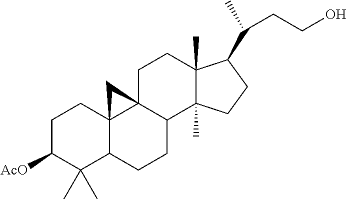

I-15

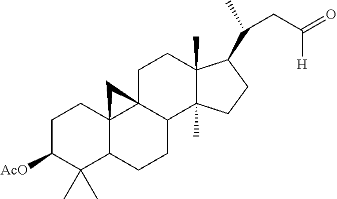

I-16

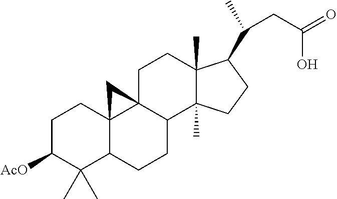

I-17

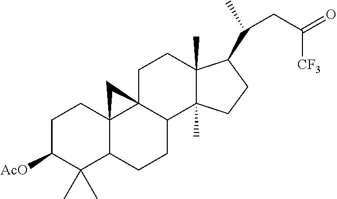

I-18

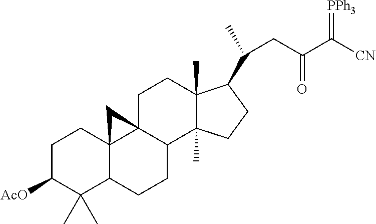

I-19

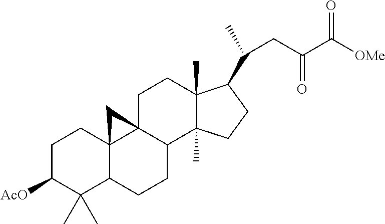

I-20

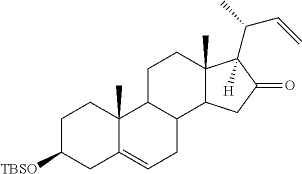

I-21

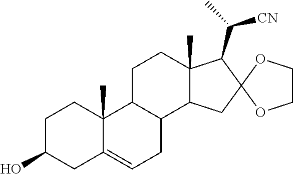

I-22

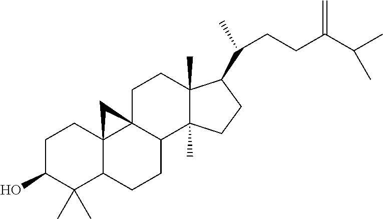

I-23

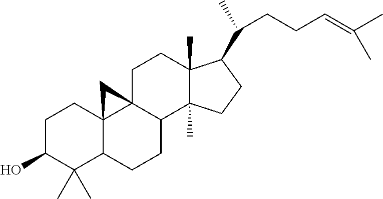

I-24

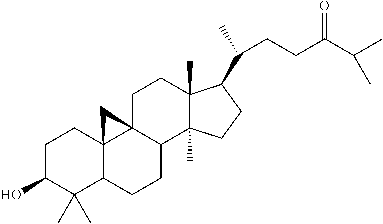

I-25

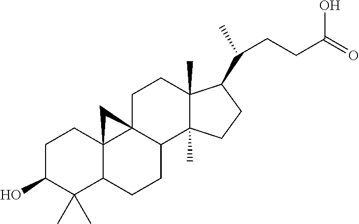

I-26

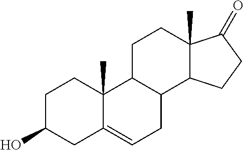

I-27

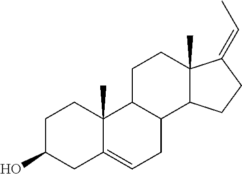

I-28

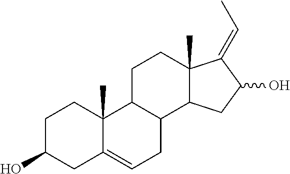

I-29

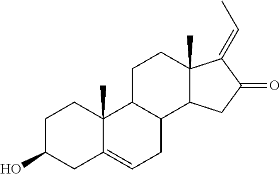

I-30

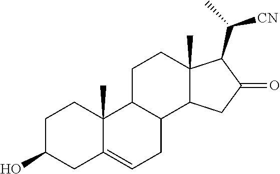

I-31

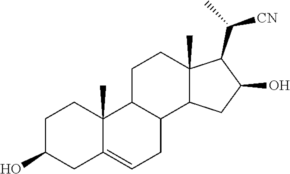

I-32

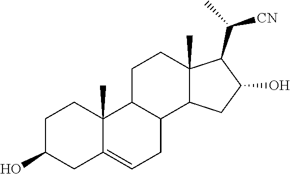

I-33

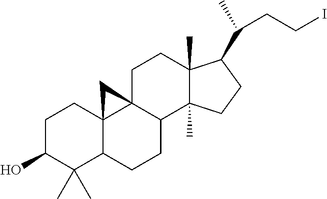

I-34

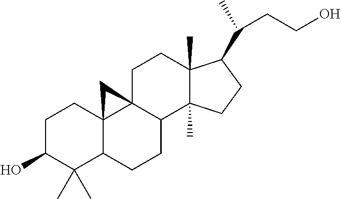

I-35

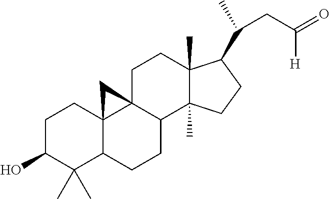

I-36

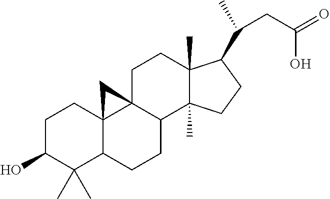

I-37

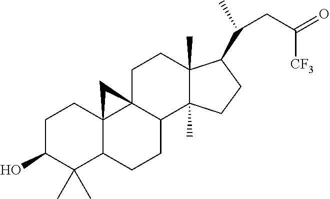

I-38

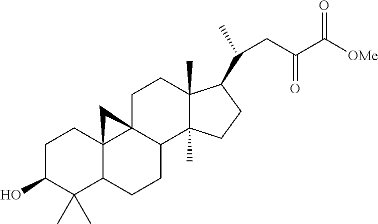

I-39

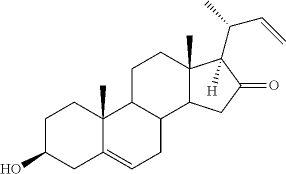

I-40

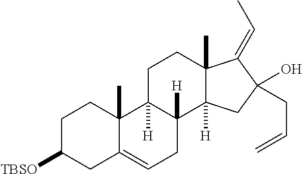

I-41

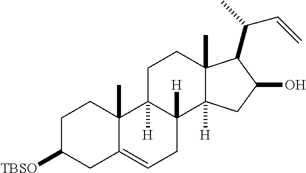

I-42

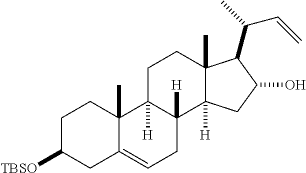

I-43

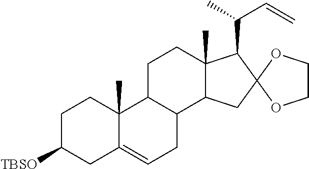

I-44

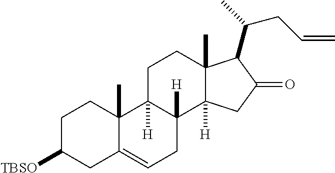

I-45

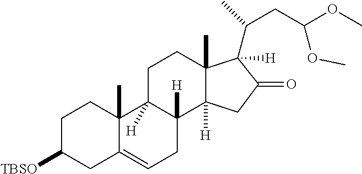

I-46

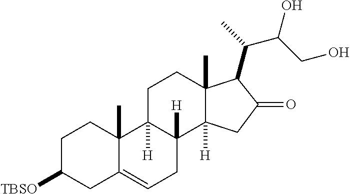

I-47

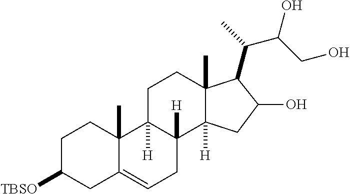

I-48

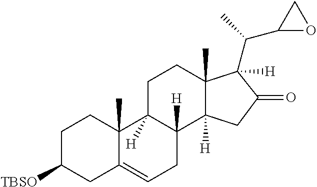

I-49

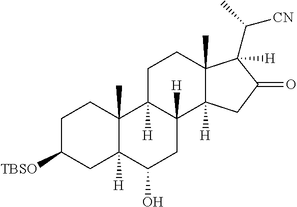

I-50

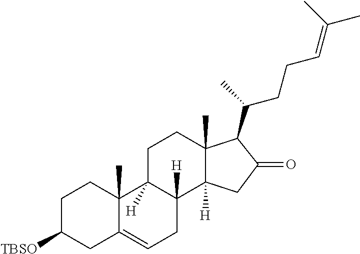

I-51

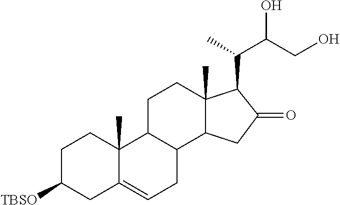

I-52

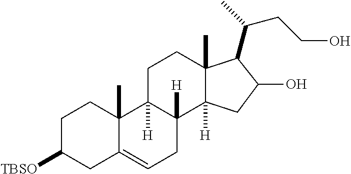

I-53

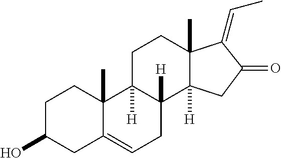

I-54

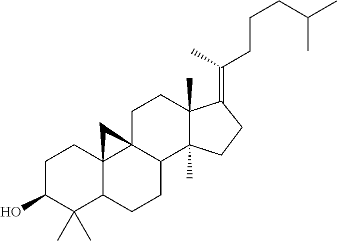

I-55

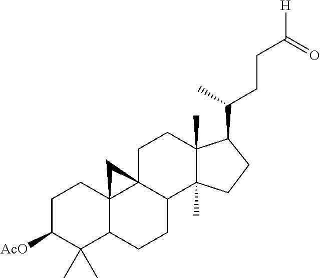

I-56

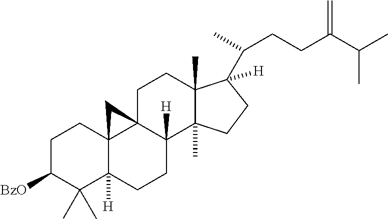

I-57

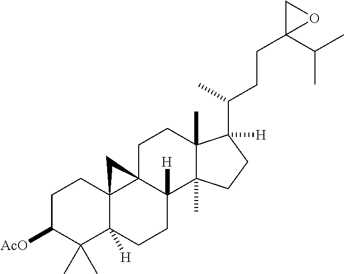

I-58

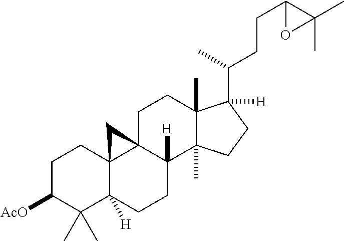

I-59

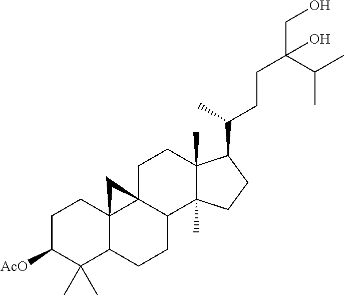

I-60

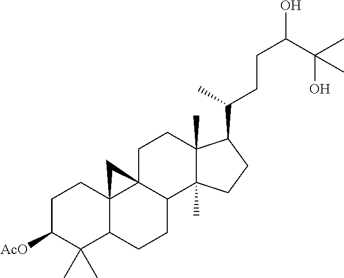

I-61

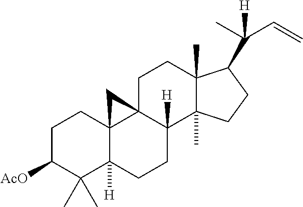

I-62

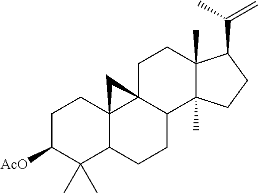

I-63

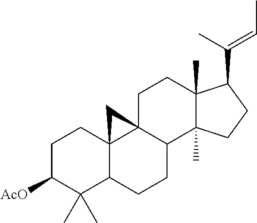

I-64

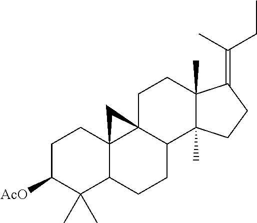

I-65

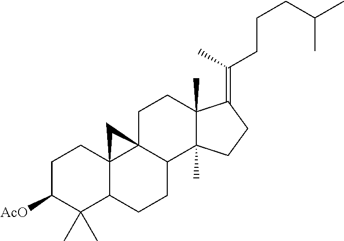

I-66

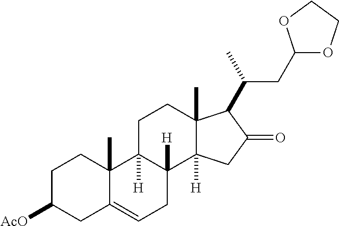

I-67

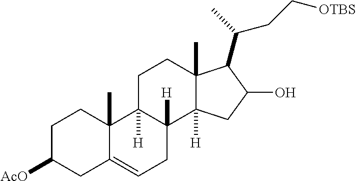

I-68

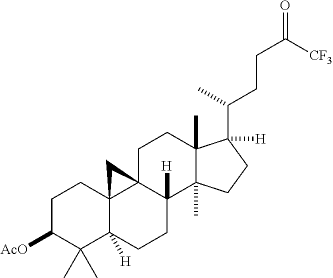

I-69

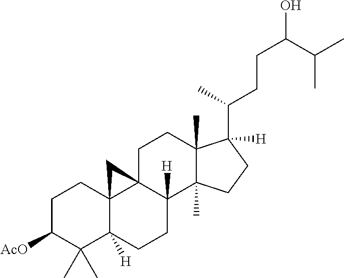

I-70

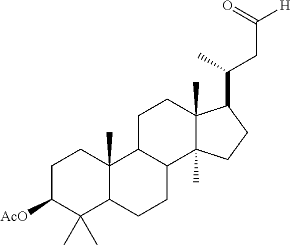

I-71

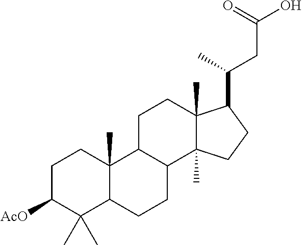

I-72

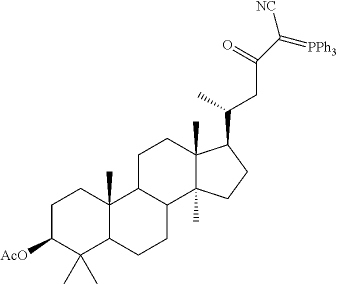

I-73

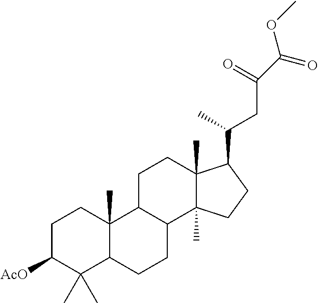

I-74

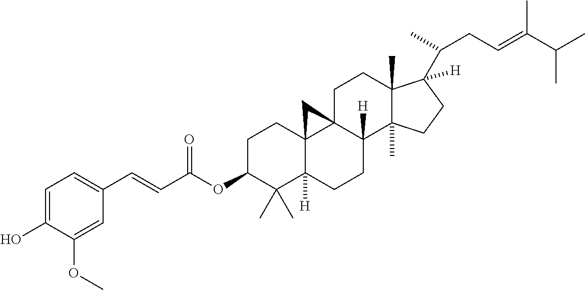

I-75

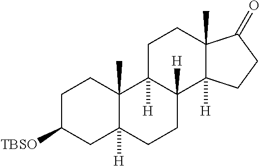

I-76

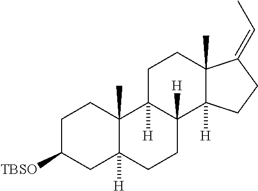

I-77

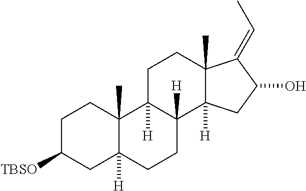

I-78

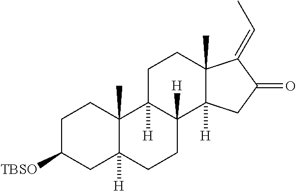

I-79

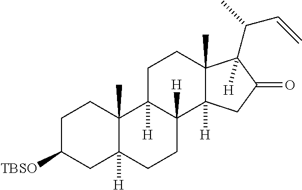

I-80

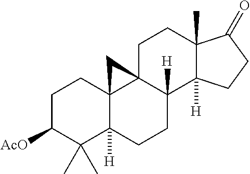

I-81

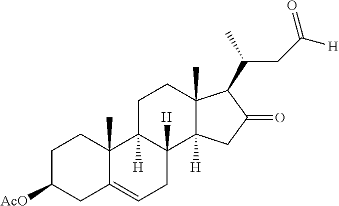

I-82

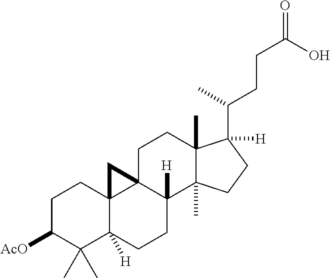

I-83

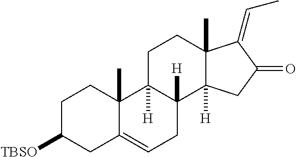

I-84

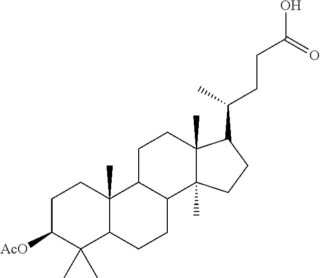

I-85

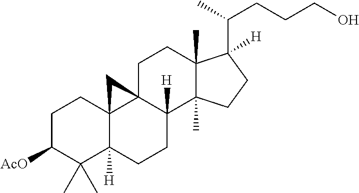

I-86

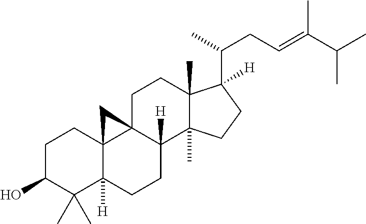

I-87

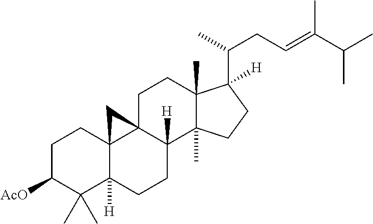

I-88

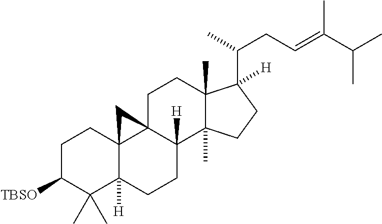

I-89

### 4. General Methods of Providing the Present Compounds

The compounds of this invention may be prepared in general by synthetic and/or semi-synthetic methods known to those skilled in the art for analogous compounds and by the schemes and methods described in detail in the Examples, below.

### 5. Uses, Formulation and Administration Pharmaceutically Acceptable Compositions

According to another aspect of the present invention, pharmaceutically acceptable compositions are provided, wherein these compositions comprise any of the compounds as described herein, and optionally comprise a pharmaceutically acceptable carrier, adjuvant or vehicle. In certain embodiments, these compositions optionally further comprise one or more additional therapeutic agents.

It will also be appreciated that certain of the compounds of present invention can exist in free form for treatment, or where appropriate, as a pharmaceutically acceptable salt thereof.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. A “pharmaceutically acceptable salt” means any non-toxic salt or salt of an ester of a compound of this invention that, upon administration to a recipient, is capable of providing, either directly or indirectly, a compound of this invention or a pharmaceutically active metabolite or residue thereof. As used herein, the term “pharmaceutically active metabolite or residue thereof” means that a metabolite or residue thereof is also a pharmaceutically active compound in accordance with the present invention.

Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, malcic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecyl sulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄ alkyl)₄ salts. This invention also envisions the quaternization of any basic nitrogen-containing groups of the compounds disclosed herein. Water or oil-soluble or dispersable products may be obtained by such quaternization. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, alkyl sulfonate and aryl sulfonate.

The compositions of the present invention may additionally comprise a pharmaceutically acceptable carrier, adjuvant, or vehicle, which, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutically acceptable compositions and known techniques for the preparation thereof. Except insofar as any conventional carrier medium is incompatible with the compounds of the invention, such as by producing any undesirable biological effect or otherwise interacting in a deleterious manner with any other component(s) of the pharmaceutically acceptable composition, its use is contemplated to be within the scope of this invention. Some examples of materials which can serve as pharmaceutically acceptable carriers include, but are not limited to, ion exchangers, alumina, aluminum stearate, lecithin, serum proteins, such as human scrum albumin, buffer substances such as phosphates, glycine, sorbic acid, or potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, polyacrylates, waxes, polyethylene-polyoxypropylene-block polymers, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The compositions provided by the present invention can be employed in combination therapies, that is the present compositions can be administered concurrently with, prior to, or subsequent to, one or more other desired therapeutic agents or medical procedures. The particular combination of therapies (therapeutic agents or procedures) to employ in a combination regimen will take into account compatibility of the desired therapeutic agents and/or procedures and the desired therapeutic effect to be achieved. It will also be appreciated that the therapies employed may achieve a desired effect for the same disorder (for example, a compound described herein may be administered concurrently with another therapeutic agent used to treat the same disorder), or they may achieve different effects (e.g., control of any adverse effects).

For example, known agents useful for treating neurodegenerative disorders may be combined with the compositions of this invention to treat neurodegenerative disorders, such as Alzheimer's disease. Examples of such known agents useful for treating neurodegenerative disorders include, but are not limited to, treatments for Alzheimer's disease such as acetylcholinesterase inhibitors, including donepezil, memantine (and related compounds as NMDA inhibitors), Exelon®; treatments for Parkinson's disease such as L-DOPA/carbidopa, entacapone, ropinrole, pramipexole, bromocriptine, pergolide, trihexephendyl, and amantadine; agents for treating Multiple Sclerosis (MS) such as beta interferon (e.g., Avonex® and Rebif), Copaxone®, and mitoxantrone; riluzole, and anti-Parkinsonian agents. For a more comprehensive discussion of updated therapies useful for treating neurodegenerative disorders, see, a list of the FDA approved drugs at http://www.fda.gov, and The Merck Manual, Seventeenth Ed. 1999, the entire contents of which are hereby incorporated by reference.

In other embodiments, the compounds of the present invention are combined with other agents useful for treating neurodegenerative disorders, such as Alzheimer's disease, wherein such agents include beta-secretase inhibitors, gamma-secretase inhibitors, aggregation inhibitors, farnesyl transferase inhibitors, metal chelators, antioxidants, and neuroprotectants.

As used herein, the term “combination,” “combined,” and related terms refers to the simultaneous or sequential administration of therapeutic agents in accordance with this invention. For example, a compound of the present invention may be administered with another therapeutic agent simultaneously or sequentially in separate unit dosage forms or together in a single unit dosage form. Accordingly, the present invention provides a single unit dosage form comprising a compound of formula I, an additional therapeutic agent, and a pharmaceutically acceptable carrier, adjuvant, or vehicle.

Other examples of agents the inhibitors of this invention may also be combined with include, without limitation: treatments for asthma such as albuterol and Singulair®; agents for treating schizophrenia such as zyprexa, risperdal, seroquel, and haloperidol; anti-inflammatory agents such as corticosteroids, TNF blockers, IL-1 RA, azathioprine, cyclophosphamide, and sulfasalazine; immunomodulatory and immunosuppressive agents such as cyclosporin, tacrolimus, rapamycin, mycophenolate mofetil, interferons, corticosteroids, cyclophosphamide, azathioprinc, and sulfasalazinc; neurotrophic factors such as acetylcholinesterase inhibitors, MAO inhibitors, interferons, anti-convulsants, ion channel blockers, agents for treating cardiovascular disease such as beta-blockers, ACE inhibitors, diuretics, nitrates, calcium channel blockers, and statins; agents for treating liver disease such as corticosteroids, cholestyramine, interferons, and anti-viral agents; agents for treating blood disorders such as corticosteroids, anti-leukemic agents, and growth factors; and agents for treating immunodeficiency disorders such as gamma globulin.

The amount of additional therapeutic agent present in the compositions of this invention will be no more than the amount that would normally be administered in a composition comprising that therapeutic agent as the only active agent. In certain embodiments, the amount of additional therapeutic agent in the present compositions will range from about 50% to 100% of the amount normally present in a composition comprising that agent as the only therapeutically active agent.

In an alternate embodiment, the methods of this invention that utilize compositions that do not contain an additional therapeutic agent, comprise the additional step of separately administering to said patient an additional therapeutic agent. When these additional therapeutic agents are administered separately they may be administered to the patient prior to, sequentially with or following administration of the compositions of this invention.

The pharmaceutically acceptable compositions of this invention can be administered to humans and other animals orally, rectally, parenterally, intracisternally, intravaginally, intraperitoneally, topically (as by powders, ointments, or drops), bucally, as an oral or nasal spray, or the like, depending on the severity of the disorder being treated. In certain embodiments, the compounds of the invention may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

Present compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

### Uses of Compounds and Pharmaceutically Acceptable Compositions

Compounds of the present invention are useful for modulating and/or inhibiting amyloid-beta (1-42) peptide production in a patient. Accordingly, the compounds of the present invention are useful for treating, or lessening the severity of, disorders associated with amyloid-beta (1-42) peptide production in a patient.

Compounds and compositions, according to the method of the present invention, may be administered using any amount and any route of administration effective for treating or lessening the severity of a neurodegenerative disorder. The exact amount required will vary from subject to subject, depending on the species, age, and general condition of the subject, the severity of the infection, the particular agent, its mode of administration, and the like.

In certain embodiments, the present invention provides a method for modulating and/or inhibiting amyloid-beta (1-42) peptide production in a patient, wherein said method comprises administering to said patient a compound of formula I, II, III, IV, or V, or a pharmaceutically acceptable composition comprising said compound. In other embodiments, the present invention provides a method of selectively modulating and/or inhibiting amyloid-beta (1-42) peptide production in a cell, wherein said method comprises administering to said patient a compound of formula I, II, III, IV, or V, or a pharmaceutically acceptable composition thereof. In still other embodiments, the present invention provides a method of reducing amyloid-beta (1-42) peptide levels in a patient, wherein said method comprises administering to said patient a compound of formula I, II, III, IV, or V, or a pharmaceutically acceptable composition thereof. In other embodiments, the present invention provides a method for reducing amyloid-beta (1-42) peptide levels in a cell, comprising contacting said cell with a compound of formula I, II, III, IV, or V. Another embodiment provides a method for reducing amyloid-beta (1-42) in a cell without substantially reducing amyloid-beta (1-40) peptide levels in the cell, comprising contacting said cell with a compound of formula I, II, III, IV, or V. Yet another embodiment provides a method for reducing amyloid-beta (1-42) in a cell and increasing at least one of amyloid-beta (1-37) and amyloid-beta (1-39) in the cell, comprising contacting said cell with a compound of formula I, II, III, IV, or V.

As used herein, the term “reducing” or “reduce” refers to the relative decrease in the amount of an amyloid-beta achieved by administering a compound of formula I, II, III, IV, or V, as compared to the amount of that amyloid-beta in the absence of administering a formula I, II, III, IV, or V. By way of example, a reduction of amyloid-beta (1-42) means that the amount of amyloid-beta (1-42) in the presence of a compound of formula I, II, III, IV, or V, is lower than the amount of amyloid-beta (1-42) in the absence of a compound of formula I, II, III, IV, or V.

In still other embodiments, the present invention provides a method for selectively reducing amyloid-beta (1-42) peptide levels in a patient, wherein said method comprises administering to said patient a compound of formula I, II, III, IV, or V, or a pharmaceutically acceptable composition thereof. In certain embodiments, the present invention provides a method for reducing amyloid-beta (1-42) peptide levels in a patient without substantially reducing amyloid-beta (1-40) peptide levels, wherein said method comprises administering to said patient a compound of formula I, II, III, IV, or V, or a pharmaceutically acceptable composition thereof.

In certain embodiments, the present invention provides a method for reducing amyloid-beta (1-42) peptide levels in a patient and increasing at least one of amyloid-beta (1-37) and amyloid-beta (1-39), wherein said method comprises administering to said patient a compound of formula I, II, III, IV, or V, or a pharmaceutically acceptable composition thereof.

The term “increasing” or “increase,” as used herein in reference to an amount of an amyloid-beta, refers to the relative rise in the amount of an amyloid-beta achieved by administering a compound of formula I, II, III, IV, or V (or contacting a cell with a compound of formula I, II, III, IV, or V) as compared to the amount of that amyloid-beta in the absence of administering a compound of formula I, II, III, IV, or V (or contacting a cell with a compound of formula I, II, III, IV, or V). By way of example, an increase of amyloid-beta (1-37) means that the amount of amyloid-beta (1-37) in the presence of a compound of formula I, II, III, IV, or V, is higher than the amount of amyloid-beta (1-37) in the absence of a compound of formula I, II, III, IV, or V. For instance, the relative amounts of either of amyloid-beta (1-37) and amyloid-beta (1-39) can be increased either by an increased production of either of amyloid-beta (1-37) and amyloid-beta (1-39) or by a decreased production of longer amyloid-beta peptides, e.g., amyloid-beta (1-40) and/or amyloid-beta (1-42). In addition, it will be appreciated that the term “increasing” or “increase,” as used herein in reference to an amount of an amyloid-beta, also refers to the absolute rise in the amount of an amyloid-beta achieved by administering a compound of formula I, II, III, IV, or V. Thus, in certain embodiments, the present invention provides a method for increasing the absolute level of at least one of amyloid-beta (1-37) and amyloid-beta (1-39), wherein said method comprises administering to said patient a compound of formula I, II, III, IV, or V, or a pharmaceutically acceptable composition thereof. In other embodiments, the present invention provides a method for increasing the level of at least one of amyloid-beta (1-37) and amyloid-beta (1-39), wherein the increase is relative to the amount of longer amyloid-beta peptides, e.g., amyloid-beta (1-40) and/or amyloid-beta (1-42), or total amyloid-beta, wherein said method comprises administering to said patient a compound of formula I, II, III, IV, or V, or a pharmaceutically acceptable composition thereof.

One of ordinary skill in the art will appreciate that overall ratio of amyloid-beta peptides is significant where selective reduction of amyloid-beta (1-42) is especially advantageous. In certain embodiments, the present compounds reduce the overall ratio of amyloid-beta (1-42) peptide to amyloid-beta (1-40) peptide. Accordingly, another aspect of the present invention provides a method for reducing the ratio of amyloid-beta (1-42) peptide to amyloid-beta (1-40) peptide in a patient, comprising administering to said patient a compound of formula I, II, III, IV, or V, or a pharmaceutically acceptable composition thereof. In certain embodiments, the ratio of amyloid-beta (1-42) peptide to amyloid-beta (1-40) peptide is reduced from a range of about 0.1 to about 0.4 to a range of about 0.05 to about 0.08.

In other embodiments, the present invention provides a method for reducing the ratio of amyloid-beta (1-42) peptide to amyloid-beta (1-40) peptide in a cell, comprising contacting the cell with a compound of formula I, II, III, IV, or V. In certain embodiments, the ratio of amyloid-beta (1-42) peptide to amyloid-beta (1-40) peptide is reduced from a range of about 0.1 to about 0.4 to a range of about 0.05 to about 0.08.

According to one aspect, the present invention provides a method for treating or lessening the severity of a disorder associated with amyloid-beta (1-42) peptide, wherein said method comprises administering to said patient a compound of formula I, II, III, IV, or V, or a pharmaceutically acceptable composition thereof. Such disorders include neurodegenerative disorders such as Alzheimer's disease (familial and sporadic), Parkinson's disease, and Down's syndrome.

Such disorders also include inclusion body myositis (deposition of A-beta in peripheral muscle, resulting in peripheral neuropathy), cerebral amyloid angiopathy (amyloid in the blood vessels in the brain), and mild cognitive impairment.

“High A-beta42” is a measurable condition that precedes symptomatic disease, especially in familial patients, based on plasma, CSF measurements, and/or genetic screening. This concept is analogous to the relationship between elevated cholesterol and heart disease. Thus, another aspect of the present invention provides a method for preventing a disorder associated with elevated amyloid-beta (1-42) peptide, wherein said method comprises administering to said patient a compound of formula I, II, III, IV, or V, or a pharmaceutically acceptable composition thereof.

In other embodiments, the present invention provides a method for treating diseases where A-beta amyloidosis may be an underlying aspect or a co-existing and exacerbating factor, wherein said method comprises administering to said patient a compound of formula I, II, III, IV, or V, or a pharmaceutically acceptable composition thereof.

In still other embodiments, the present invention provides a method for treating a disorder in a patient, wherein said method comprises administering to said patient a compound of formula I, II, III, IV, or V, or a pharmaceutically acceptable composition thereof, and wherein said disorder is Lewy body dementia (associated with deposition of alpha-synuclein into Lewy bodies in cognitive neurons; α-synuclein is more commonly associated with deposits in motor neurons and the etiology of Parkinson's disease), Parkinson's disease, cataract (where a-beta is aggregating in the eye lens), Tauopathies (e.g. frontotemporal dementia), Huntington's disease, ALS/Lou Gerhig's disease, Type 2 diabetes (IAPP aggregates in pancreatic islets, is similar in size and sequence to A-beta and having type 2 diabetes increases risk of dementia), Transthyretin amyloid disease (TTR, an example of this disease is in heart muscle contributing to cardiomyopathy), prion disease, and Creutzfeldt-Jakob disease (CJD).

In other embodiments, the present invention provides a method for treating or lessening the severity of Alzheimer's disease in a patient, wherein said method comprises administering to said patient a compound of formula I, II, III, IV, or V, or a pharmaceutically acceptable composition thereof.

Without wishing to be bound by any particular theory, it is believed that the present compounds are modulators of gamma-secretase which selectively reduce levels of amyloid-beta (1-42). Accordingly, another embodiment of the present invention provides a method of modulating gamma-secretase in a patient, comprising administering to said patient a compound of formula I, II, III, IV, or V, or pharmaceutically acceptable composition thereof. In certain embodiments, the present compounds are inhibitors of gamma-secretase. Said method is useful for treating or lessening the severity of any disorder associated with gamma-secretase. Such disorders include, without limitation, neurodegenerative disorders, e.g. Alzheimer's disease.

The Notch/Delta signaling pathway is highly conserved across species and is widely used during both vertebrate and invertebrate development to regulate cell fate in the developing embryo. See Gaiano and Fishell, “The Role of Notch in Promoting Glial and Neural Stem Cell Fates” Annu. Rev. Neurosci. 2002, 25:471-90. Notch interacts with the gamma-secretase complex and has interactions with a variety of other proteins and signaling pathways. Notchl competes with the amyloid precursor protein for gamma-secretase and activation of the Notch signaling pathway down-regulates PS-1 gene expression. See Lleo et al., “Notch1 Competes with the Amyloid Precursor Protein for γ-Secretase and Down-regulates Presenilin-1 Gene Expression” Journal of Biological Chemistry 2003, 48:47370-47375. Notch receptors are processed by gamma-secretase acting in synergy with T cell receptor signaling and thereby sustain peripheral T cell activation. Notchl can directly regulate Tbx21 through complexes formed on the Tbx21 promoter. See Minter et al., “Inhibitors of γ-secretase block in vivo and in vitro T helper type 1 polarization by preventing Notch upregulation of Tbx21,” Nature Immunology 2005, 7:680-688. In vitro, gamma-secretase inhibitors extinguished expression of Notch, interferon-gamma and Tbx21 in TH1-polarized CD4+ cells. In vivo, administration of gamma-secretase inhibitors substantially impeded TH1-mediated disease progression in the mouse experimental autoimmune encephalomyelitis model of multiple sclerosis suggesting the possibility of using such compounds to treat TH1-mediated autoimmunity See Id. Inhibition of gamma-secretase can alter lymphopoiesis and intestinal cell differentiation (Wong et al., “Chronic Treatment with the γ-Secretase Inhibitor LY-411,575 Inhibits β-Amyloid Peptide Production and Alters Lymphopoiesis and Intestinal Cell Differentiation” Journal of Biological Chemistry 2004, 26:12876-12882), including the induction of goblet cell metaplasia. See Milano et al., “Modulation of Notch Processing by γ-Secretase Inhibitors Causes Intestinal Goblet Cell Metaplasia and Induction of Genes Known to Specify Gut Secretory Lineage Differentiation” Toxicological Sciences 2004, 82:341-358.

Strategies that can alter amyloid precursor protein (“APP”) processing and reduce the production of pathogenic forms of amyloid-beta without affecting Notch processing are highly desirable. Moreover, as described above, the inhibition of gamma-secretase has been shown in vitro and in vivo to inhibit the polarization of Th cells and is therefore useful for treating disorders associated with Th1 cells. Th1 cells are involved in the pathogenesis of a variety of organ-specific autoimmune disorders, Crohn's disease, Helicobacter pylori-induced peptic ulcer, acute kidney allograft rejection, and unexplained recurrent abortions, to name a few.

According to one embodiment, the invention relates to a method of inhibiting the formation of Th1 cells in a patient comprising the step of administering to said patient a compound of the present invention, or a composition comprising said compound. In certain embodiments, the present invention provides a method for treating one or more autoimmune disorders, including irritable bowel disorder, Crohn's disease, rheumatoid arthritis, psoriasis, Helicobacter pylori-induced peptic ulcer, acute kidney allograft rejection, multiple sclerosis, or systemic lupus erythematosus, wherein said method comprises administering to said patient a compound of formula I, II, III, IV, or V, prepared according to methods of the present invention, or a pharmaceutically acceptable composition comprising said compound.

In certain embodiments, the present invention provides a method for modulating and/or inhibiting amyloid-beta peptide production, without affecting Notch processing, in a patient, wherein said method comprises administering to said patient a compound of formula I, II, III, IV, or V, or a pharmaceutically acceptable composition comprising said compound.

In certain embodiments, the present invention provides a method for inhibiting amyloid-beta (1-42) peptide production, without affecting Notch processing, in a patient, wherein said method comprises administering to said patient a compound of formula I, II, III, IV, or V, or a pharmaceutically acceptable composition comprising said compound.

In certain embodiments, the present invention provides a method for reducing amyloid-beta (1-42) peptide levels in a patient and increasing at least one of amyloid-beta (1-37) and amyloid-beta (1-39), without affecting Notch processing, wherein said method comprises administering to said patient a compound of formula I, II, III, IV, or V, or a pharmaceutically acceptable composition thereof.

Accordingly, another aspect of the present invention provides a method for reducing the ratio of amyloid-beta (1-42) peptide to amyloid-beta (1-40) peptide in a patient, without affecting Notch processing, comprising administering to said patient a compound of formula I, II, III, IV, or V, or a pharmaceutically acceptable composition thereof. In certain embodiments, the ratio of amyloid-beta (1-42) peptide to amyloid-beta (1-40) peptide is reduced from a range of about 0.1 to about 0.4 to a range of about 0.05 to about 0.08.

The compounds of the invention are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of the present invention will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts. The term “patient,” as used herein, means an animal, preferably a mammal, and most preferably a human.

### EXEMPLIFICATION

The invention is further illustrated by the following examples which are not intended to be limiting. The reagents used in the reactions described below are either commercially available or those whose synthesis would be readily known to those skilled in the art. Methods and intermediates for preparing compounds of the present invention include those known to one of ordinary skill in the art. One of ordinary skill in the art will appreciate that the Examples and Schemes set forth below can be readily modified to prepare other compounds of the present invention.

Compound numbers utilized below correspond to the compound numbers recited in Table 1, supra.

General comments. Melting points are uncorrected. ¹H NMR spectra were measured at 400 MHz in CDCl₃. Chemical shifts are downfield from trimethylsilane (TMS) as internal standards, and J values are in hertz. Mass spectra were obtained on API-2000, with ESI technique. All solvents used were reagent grade. Dichloromethane (DCM) was freshly distilled over CaH₂. Tetrahydrofuran (THF) and diethyl ether were freshly distilled over sodium-benzophenone ketyl. Gamma-oryzanol was purchased from ChemPacific Corporation (Baltimore, Md., USA).

Other abbreviations include: Ac₂O (acetic anhydride), DMAP (dimethylaminopyridine), PhI(OAc)₂ (iodosobenzene diacetate), PDC (pyridinium dichromate), TFAA (trifluoroacetic acid), DMDO (dimethyldioxirane), RB (round-bottom), TLC (thin layer chromatography), MeOH (methanol), MeOD (methanol d-4), i-PrOH (isopropanol), TBDMS (tert-butyldimethylsilyl-), TBS (tert-butyldimethylsilyl-), DHEA (dehydroepiandrosterone), TBHP (tert-butylhydroperoxide), DMSO (dimethylsulfoxide), KOt-Bu (potassium tert-butoxide), MS (mass spectrometry), EtOAc (ethyl acetate), M.P. (melting point), EtPPh₃I (ethyltriphenylphosphonium iodide), Et₃N (triethyl amine), mCPBA (meta-chloroperbenzoic acid), BF₃.OEt₂ (trifluoroborane etherate), EtOH (ethanol), RT (room temperature), NMR (nuclear magnetic resonance).

### Example 1

Compounds I-1, I-2, I-3, and I-4 were prepared according to the scheme below.

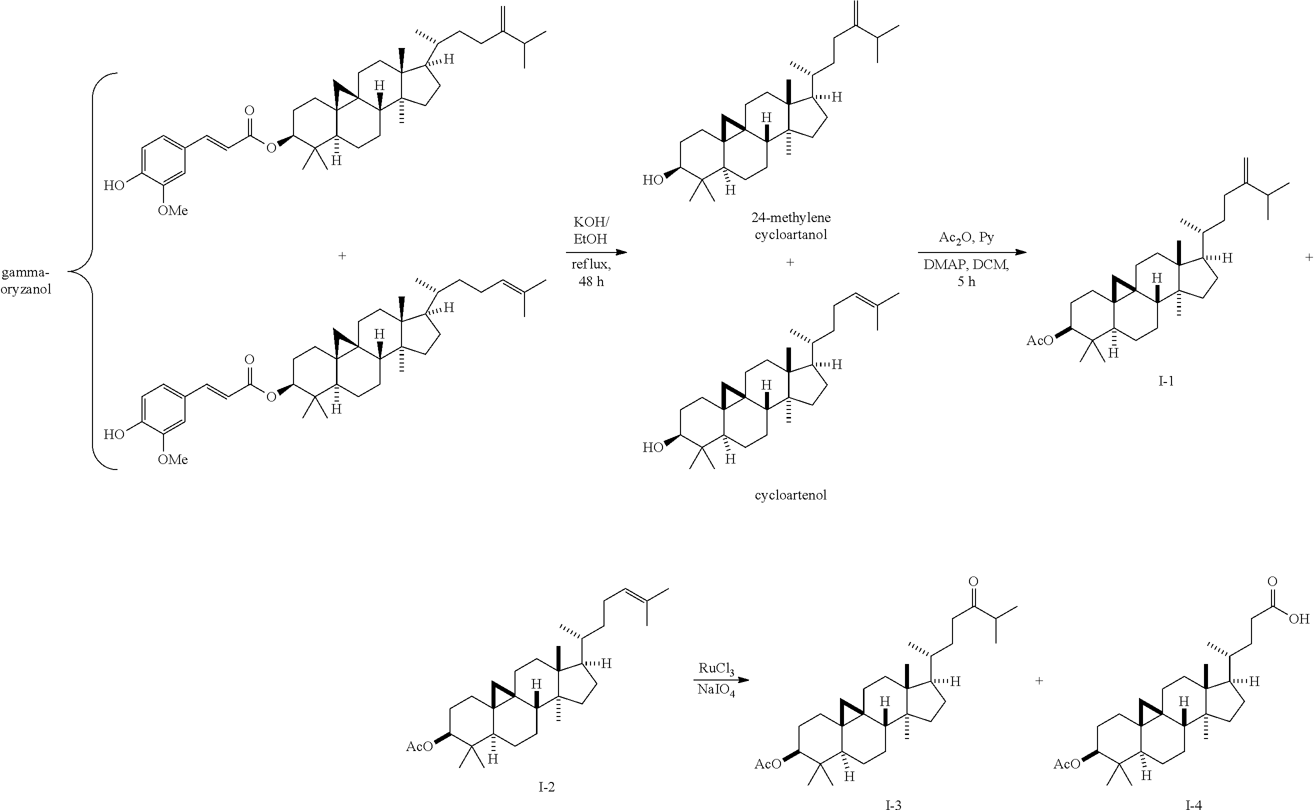

### 24-Methylene cycloartanol and cycloartenol

To a stirred solution of and 24-methylene cycloartanol-3-ferulate ester and cycloartenol-3-ferulate ester (gamma-oryzanol, 5 g, 8.11 mmol) in EtOH (35 mL) was added 10 N KOH in EtOH (8.4 mL) at RT. The resulting reaction mixture was warmed to 80° C. and stirred for 48 h. The reaction mixture was cooled to room temperature and the solvent was reduced in vacuo until the appearance of a white precipitate was observed. The precipitate was isolated by vacuum filtration and the resulting cake was washed with water (4×50 mL) and then dissolved in ether (40 mL). The organic solution was then washed with water (2×20 mL), dried (Na₂SO₄), and reduced in vacuo. The product was purified by silica gel chromatography [EtOAc/Hexane (1:4)] to give a mixture of 24-methylene cycloartanol and cycloartenol as a white solid (2.6 g, 73%); mp 98-100° C. ¹H NMR (δ): 0.32 (d, J=4.1, 2H), 0.54 (d, J=4.1, 2H), 0.72-0.84 (m, 7H), 0.84-0.91 (m, 11H), 0.95 (br s, 10H), 1.01 (d, J=2.2, 3H), 1.02 (d, J=2.0, 3H), 1.05-1.17 (m, 5H), 1.19-1.35 (m, 13H), 1.35-1.43 (m, 2H), 1.44-1.65 (m, 20H), 1.67 (s, 3H), 1.71-1.77 (m, 2H), 1.80-2.08 (m, 7H), 2.08-2.18 (m, 1H), 2.18-2.28 (m, 1H), 3.25-3.32 (m, 2H), 4.65 (s, 1H), 4.70 (s, 1H), 5.09 (br t, J=7.0, 1H). MS (m/z): 441 & 427 (M+H)⁺.

### 3-O-Acetyl-24-methylene cycloartanol (I-1) and 3-O-acetylcycloartenol (I-2)

To a stirred solution of 24-methylene cycloartanol and cycloartenol (5 g, 11.34 mmol) in dry pyridine (25 mL), was added a catalytic amount of DMAP (14 mg) followed by the drop-wise addition of Ac₂O (1.6 mL, 17 mmol). The solution was then stirred for 4 h before being diluted with ether (25 mL), cooled to 0° C., and then quenched with aq. 2 N HCl (75 mL). The organic layer was separated and washed with satd. CuSO₄ (2×25 mL), dried (Na₂SO₄), and reduced in vacuo. The product was purified by silica gel chromatography [EtOAc/Hexane (1:9)] to give a mixture of 3-O-Acetyl-24-methylene cycloartanol (I-1) and 3-O-acetylcycloartenol (I-2) as a white solid (4.8 g, 88%); mp 102-104° C. ¹H NMR (δ): 0.33 (d, J=4.1, 2H), 0.56 (d, J=4.1, 2H), 0.70-0.93 (m, 24H), 0.94 (s, 3H), 0.95 (s, 3H), 1.01 (d, J=2.3, 3H), 1.02 (d, J=2.3, 3H), 1.06-1.13 (m, 5H), 1.18-1.42 (m, 14H), 1.46-1.61 (m, 18H), 1.67 (br s, 3H), 1.69-1.80 (m, 2H), 1.80-2.02 (m, 6H), 2.04 (br s, 6H), 2.05-2.19 (m, 1H), 2.18-2.26 (m, 1H), 4.55 (dd, J=5.0, 10.7, 2H), 4.65 (s, 1H), 4.70 (s, 1H), 5.06-5.12 (m, 1H). MS (m/z): 505 & 491 (M+Na)⁺.

### 24-Oxocycloartanol-3-β-O-acetate (I-3) and 3-β-O-acetyl-25,26,27-Trinorcycloartan-24-oic acid (I-4)

To a stirred solution of 3-O-Acetyl-24-methylene cycloartanol (I-1) and 3-O-acetylcycloartenol (I-2) (1 g, 2.07 mmol) in CHCl₃/CH₃CN (1:1, 40 mL) was added a solution of NaIO₄ (2.3 g, 16.6 mmol) in H₂O (25 mL) followed by the addition of RuCl₃ (43 mg, 0.2 mmol). The reaction mixture was stirred for 48 h and then vacuum filtered through a pad of Celite. The Celite bed was washed with CH₂Cl₂ and the organic and aqueous filtrates were separated. The aqueous filtrate was extracted with CH₂Cl₂ (3×20 mL) and the combined organic solutions were washed with brine (50 mL), dried (Na₂SO₄), and reduced in vacuo. The product was purified by silica gel chromatography [EtOAc/Hexane (1:4)] to give the (I-3) (470 mg, 46%) and (I-4) (86 mg, 9%) as separate white solids. (I-3); mp 119-121° C. ¹H NMR (δ): 0.33 (d, J=4.1, 1H), 0.56 (d, J=4.1, 1H), 0.74-0.86 (m, 7H), 0.87 (s, 3H), 0.88 (s, 3H), 0.94 (s, 3H), 1.07 (s, 3H), 1.08 (s, 3H), 1.02-1.12 (m, 2H), 1.16-1.40 (m, 9H), 1.45-1.62 (m, 6H), 1.70-1.80 (m, 2H), 1.85-2.00 (m, 2H), 2.04 (s, 3H), 2.32-2.43 (m, 1H), 2.43-2.53 (m, 1H), 2.60 (septet, J=6.9, 1H), 4.54 (dd, J=5.6, 10.9, 1H). MS (m/z): 507 (M+Na)⁺. (I-4); mp 210-212° C. ¹H NMR (δ) (MeOD): 0.40 (d, J=4.2, 1H), 0.60 (d, J=4.2, 1H), 0.8-1.02 (m, 16H), 1.1-1.21 (m, 2H), 1.22-1.49 (m, 9H), 1.5-1.78 (m, 9H), 1.80-1.87 (m, 1H), 1.90-2.10 (m, 2H), 2.02 (s, 3H), 2.15-2.24 (m, 1H), 2.28-2.29 (m, 1H), 4.50-4.54 (m, 1H).

Alternatively, this transformation was achieved by treatment with ozone in CH₂Cl₂-HCO₂H (25:1 v/v), followed by oxidative workup with Jones reagent.

### Example 2

Using the methods described in Example 1, compound I-3 was prepared. Compound I-70 was then prepared according to the scheme below.

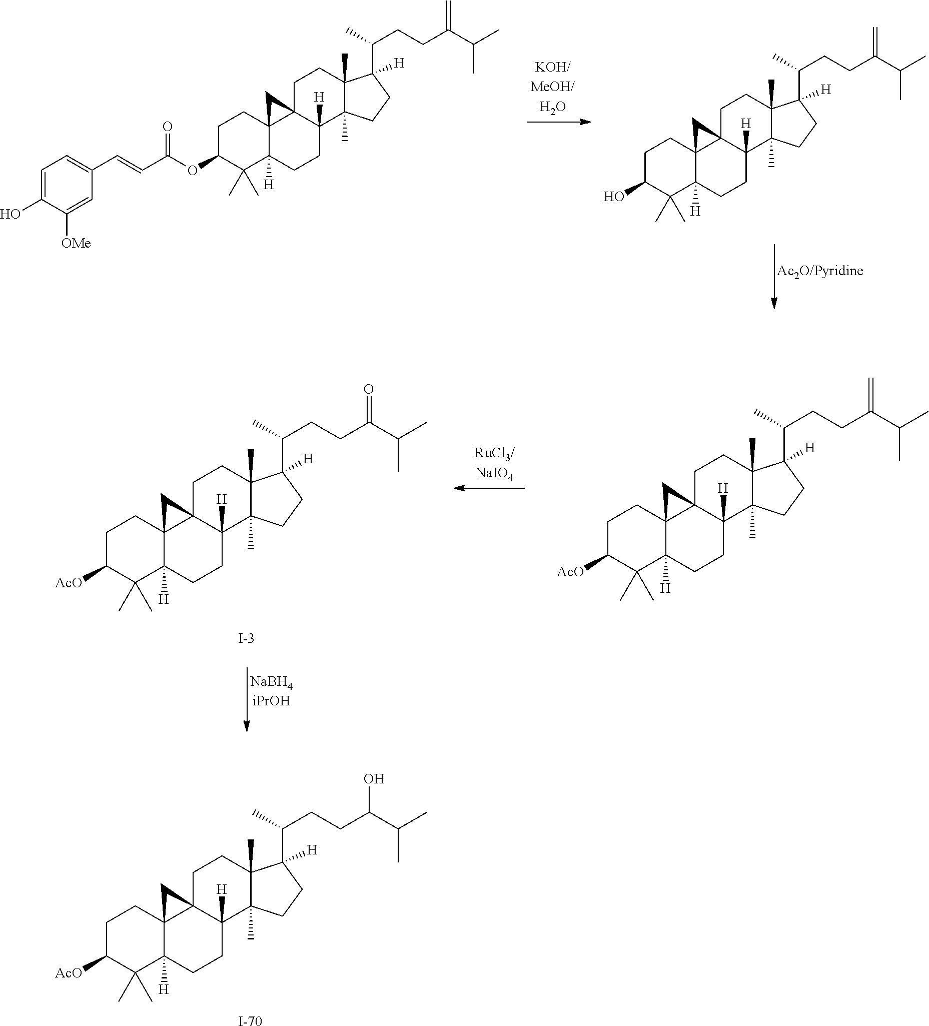

### 24-Hydroxy cycloartanol-3-β-O-acetate (I-70)

To a stirred solution of 24-oxocycloartanol-3-β-O-acetate (I-3) (100 mg, 0.2 mmol) in i-PrOH (10 mL) at 0° C. was added NaBH₄ (24 mg, 6.2 mmol). The reaction mixture was stirred for 12 h at RT and the solution was then quenched by the slow addition of ice-cold H₂O (10 mL). The solution was further diluted with EtOAc (25 mL) and the organic and aqueous layers were separated. The aqueous layer was extracted with EtOAc (3×10 mL) and the combined organic layers were dried (Na₂SO₄) and reduced in vacuo. The product was purified by silica gel chromatography [EtOAc/Hexane (1:4)] to give 24-hydroxy cycloartanol-3-acetate I-70 as a white solid (75 mg, 75%); mp 126-128° C. ¹H NMR (δ): 0.33 (d, J=4.0, 1H), 0.56 (d, J=4.0, 1H), 0.74-0.98 (m, 25H), 1.00-1.80 (m, 20H), 1.84-2.04 (m, 2H), 2.04 (s, 3H), 3.26-3.37 (m, 1H), 4.55 (dd, J=5.4, 10.3, 1H). MS (m/z): 509 (M+Na)⁺.

### Example 3

Using the methods described in Example 1, I-4 was prepared. Compounds I-14 and I-15 were then prepared according to the scheme below.

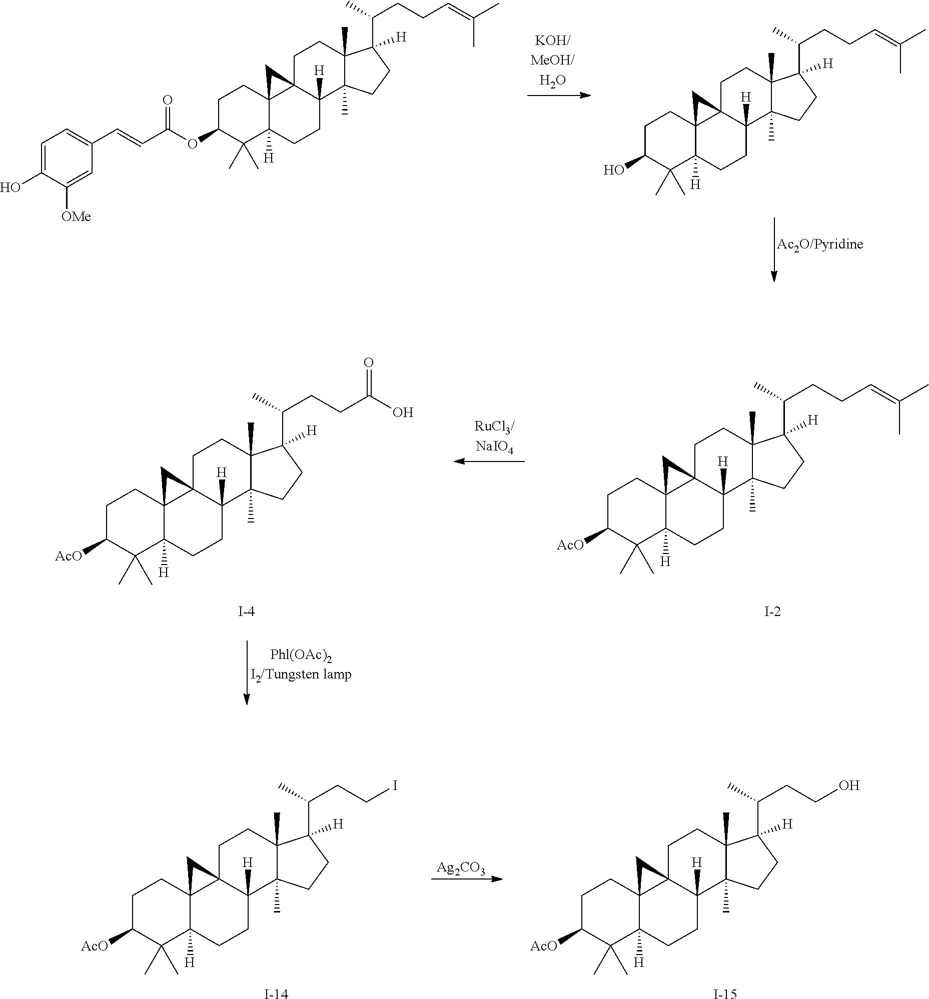

### 3β-Acetoxy-9,19-Cyclo-4,4,14-trimethyl-24-norchol-23-iodo (I-14)

To a stirred solution of acid I-4 (200 mg, 0.43 mmol) in CCl₄ (40 mL), were added in succession, iodine (121 mg, 0.47 mmol) and iodosobenzene diacetate (130 mg, 0.52 mmol). The resulting reaction mixture was irradiated with a 200 W tungsten bulb for about 12 h. The lamp was proximate enough to reflux the reaction mixture. After 12 h, the same quantities of iodine and iodosobenzene diacetate were added and the mixture irradiated for another 12 h following which it was cooled to RT and diluted with a satd. solution of Na₂S₂O₃ (25 mL) and the layers were separated. The organic layer was once again washed with Na₂S₂O₃ (25 mL) and dried (Na₂SO₄) and reduced in vacuo. The product was purified by silica gel chromatography [EtOAc/Hexane (1:19)] to give the iodide I-14 as a white solid (125 mg, 54%); mp 147-149° C.; MS (m/z): 563 (M+NH₄)¹.

### 3β-Acetoxy-9,19-Cyclo-4,4,14-trimethyl-24-norchol-23-ol (I-15)

To a vigorously stirred solution of iodide I-14 (125 mg, 0.23 mmol) in acetone/water (19:1) was added Ag₂CO₃ (127 mg, 0.46 mmol). The resulting mixture was stirred at RT for about 30 min before being refluxed overnight. This mixture was then cooled to RT and filtered through a short pad of anhyd. Na₂SO₄. The filter bed was washed with ether (60 mL). The combined filtrate was concentrated and purified by silica gel chromatography [EtOAc/Hexane (1:9)] to give alcohol I-15 as a white solid (60 mg, 60%); mp 136-138° C.; MS (m/z): 453(M+Na)⁺.

### Example 4

Compound I-17 was prepared directly from compound I-15 according to the scheme below. Alternatively, compounds I-16 and I-17 may be prepared from compound I-15 in a step-wise fashion by oxidation with PDC followed by treatment with Jones reagent.

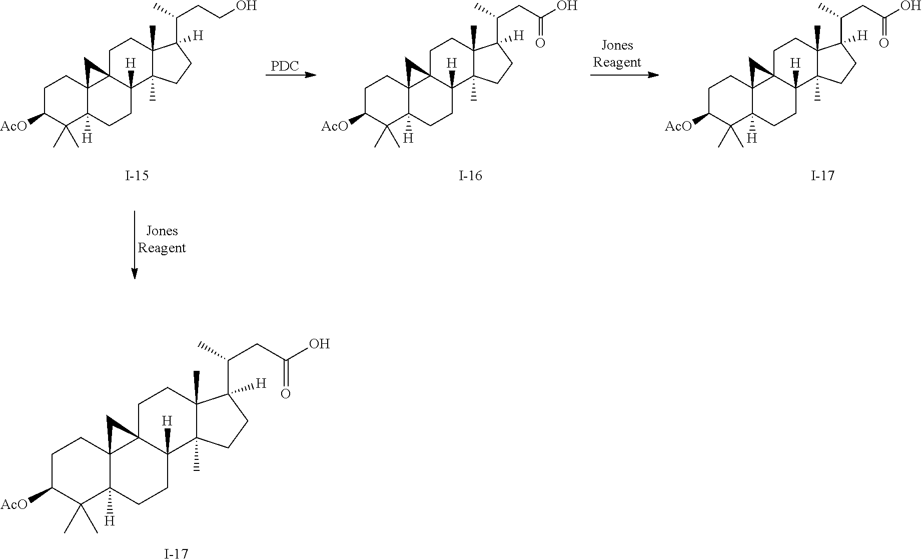

### 3β-Acetoxy-9,19-Cyclo-4,4,14-trimethyl-24-norchol-23-oic acid (I-17)

To a stirred solution of alcohol I-15 (50 mg, 0.11 mmol) in acetone (15 mL) was added in a drop-wise fashion Jones reagent until the yellow color persisted. The resulting solution was stirred for an additional 20 min during which the consumption of the starting material was found to be complete. The reaction was quenched by the addition of a large excess of i-PrOH (50 mL). This mixture was concentrated in vacuo and the green mass was purified by silica gel chromatography [EtOAc/Hexane (1:3)] to yield acid I-17 as a white solid (45 mg, 88%); mp 222-224° C.; MS (m/z): 443(M−H)¹.

### Example 5

The scheme below depicts the reduction of compound I-16 to form the hydroxyl compound I-15.

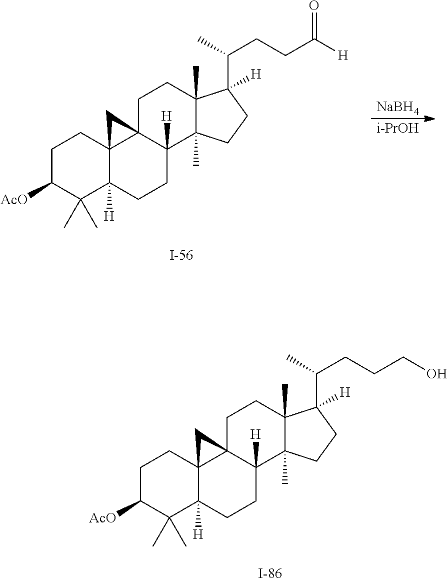

### 3β-Acetoxy-9,19-Cyclo-4,4,14-trimethyl-24-oxo-cholane (I-86)

To a stirred solution of I-56 (100 mg, 0.22 mmole) in i-PrOH (10 mL) was added in portions NaBH₄ (25 mg, 0.67 mmole), at 0° C. The resulting solution was allowed to stir overnight, after which time it was quenched by the addition of a ice-cold water (20 mL). This mixture was diluted with EtOAc (15 mL) and the layers were separated. The aqueous phase was extracted with EtOAc (2×15 mL) and the combined organic extracts was dried (Na₂SO₄) and concentrated. Silica gel chromatography [EtOAc/Hexane (1:4)] yielded I-86 as a white solid (75 mg, 75%); mp 144-146° C. MS (m/z): 462 (M+Na)⁺.

### Example 6

Compound I-18 was prepared from I-17 according to the scheme below.

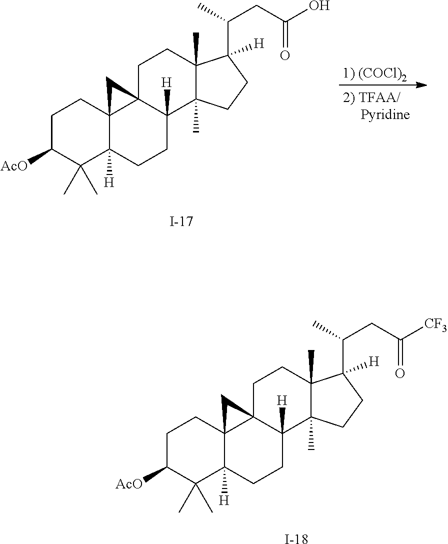

### 3β-Acetoxy-9,19-Cyclo-4,4,14-trimethyl-23-oxo-24-trifluorocholane (I-18)

This compound was prepared from acid I-17 using the method of Wong, M-K et al. J. Org. Chem. 2003, 68:6321-6328. Investigation on the Regioselectivities of Intramolecular Oxidation of Unactivated C—H Bonds by Dioxiranes Generated in situ.

### Example 7

Compounds I-19 and I-20 were prepared from compound I-17 as depicted below.

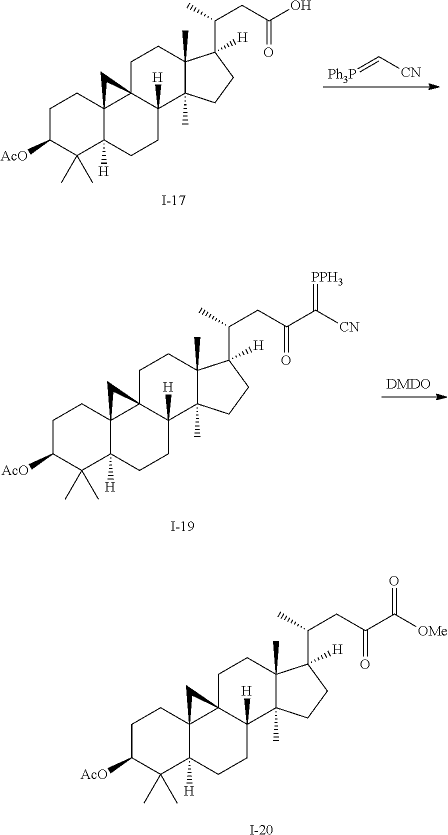

### 3β-Acetoxy-4,4,13,14-tetramethyl-tetradecahydro-cyclopropa[9,10]cyclopenta[a]phenanthren-17-yl)-2-oxo-pentanoic acid methyl ester (I-20)

Acid I-17 was converted to the corresponding cyanophosphorane ketone using the literature method [Wong, Man-Kin; Chung, Nga-Wai; He, Lan; Wang, Xue-Chao; Yan, Zheng; Tang, Yeung-Chiu; Yang, Dan. Journal of Organic Chemistry 2003, 68: 6321-6328; Yang, Dan; Wong, Man-Kin; Wang, Xue-Chao; Tang, Yeung-Chiu. J. Am. Chem. Soc. 1998, 120: 6611-6612; the entirety of each of which is incorporated herein by reference] and then oxidized to obtain methoxycarbonyl ketone I-20. A 100 ml two-necked RB flask equipped with gas out-let and solid addition bulb funnel was charged with 3.2 gm of NaHCO₃ in 5.4 ml of water and 3.5 ml of acetone. Cautiously, 6.7 gm of oxonc was added through solid addition bulb funnel in very small portions. DMDO liberated was collected/trapped in a 100 ml oven dried 2-necked RB flask equipped with gas inlet and N₂ assembly at −78° C. To the resultant solution of DMDO was added drop-wise a solution of 5 ml methanol containing 70 mg of cyanophosphorane at −78° C. over a period of 15 min. Reaction mixture was allowed to warm to ambient temperature and stirred over-night. The progress of the reaction was monitored by analytical silica gel TLC plate using 20% ethyl acetate in n-hexane, visualized in Hanessian's stain, R_{f} observed for required compound I-20 was 0.8 and 0.1 for starting material I-19. After completion of reaction, solvent was evaporated under reduced pressure. Crude product was purified by silica-gel (#100-200) column chromatography; the pure product I-20, was eluted with 4% ethyl acetate in n-hexane as a white solid, 30 mg (yield: 64%), M.P: 120-121° C. (un-corrected).

### Example 8

Compounds I-76, I-77, I-78, I-79 and I-80 were prepared from androsterone as depicted below.

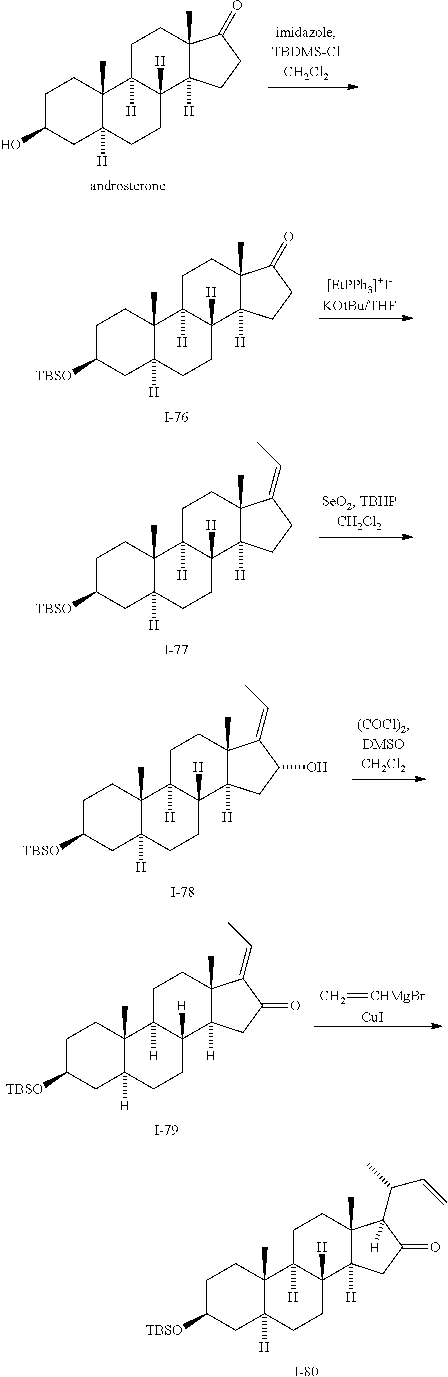

### 3-O-t-Butyldimethylsilyl-androsterone (I-76)

A 50 ml flask was charged with androsterone (0.50 g, 1.72 mmol) and dichloromethane (5 ml), the resulting solution was cooled to 0° C. and imidazole (0.28 g, 4.12 mmol) was added in small portions. Reaction mixture was stirred for 10 min and followed by addition of TBDMS-Cl (0.311 g, 2.06 mmol) in small portions. Catalytic amount of DMAP (0.021 g, 0.172 mmol) was added and resulting mixture was allowed to warm to ambient temperature and further stirred for 12 hrs. Water (10 ml) was added to the reaction mixture and extracted with CH₂Cl₂ (3×10 ml). Combined organic portion was washed with water (10 ml), brine (10 ml), dried over sodium sulfate, filtered and concentrated under reduced pressure. Crude product obtained was purified by silica gel (60-120 mesh) column chromatography eluting with ethyl acetate:hexane (1:99). Yield, 0.503 gm (72.2%). TLC: Ethyl acetate-hexane (10:90). Rf, 0.4. M.P.-159-162° C.

### 3-t-Butyldimethylsilyloxy-pregn-17(20)-ene (I-77)

A two neck 50 ml flask was charged with t-BuOK (2.21 g, 19.72 mmol) and dry THF (20 ml) and the resulting solution was cooled to 0° C. and treated with EtPPh₃I (6.022 g, 14.4 mmol) in small portions over a period of 10 min. Resulting mixture was warmed to ambient temperature and stirred for 2 h, followed by treatment with a solution of compound I-76 (1 g, 2.4 mmol) in dry THF (10 ml) in small portions over a period of 10 min. Reaction mixture was warmed to 55° C. and allowed to stir at the same temp overnight. Solvent was evaporated under reduced pressure and residue obtained was triturated with hexane (5×100 ml) Combined hexane portions were concentrated to obtain crude product, which was purified by column chromatography on silica gel (100-200 mesh) eluting with ethyl acetate-hexane (1:99). Yield, 0.724 g (70%). TLC: Ethyl acetate-hexane (5:95). Rf, 0.6. M.P.: 136-137° C.

### 3-t-Butyldimethylsilyloxy-16-hydroxy-pregn-17(20)-ene (I-78)

A two neck 25 ml flask was charged with SeO₂ (27 mg, 0.244 mmol) and CH₂Cl₂ (5 ml) and mixture cooled to 0° C. tert-butyl hydroperoxide (0.062 ml, 7.7 M) was added in small portion over a period of 5 min. The resulting mixture was stirred at 0° C. until the SeO₂ dissolved completely. This was followed by addition of a solution of compound I-77 (0.200 g, 0.479 mmol) in CH₂Cl₂ to the reaction mixture. The resulting solution was stirred at ambient temperature for 3 hrs. Water (5 ml) was added to the reaction mixture which was then extracted with CH₂Cl₂ (2×10 ml). Combined dichloromethane portions were washed with water (2×10 ml), brine (10 ml), dried over sodium sulfate and concentrated to obtain crude product which was purified by silica gel (100-200 mesh) column chromatography eluting with ethyl acetate-hexane (5:95). Yield, 0.153 g (74.0%). TLC: Ethyl acetate-hexane (20:80). Rf, 0.6. M.P.: 146-147° C.

In certain embodiments, the SeO₂-mediated oxidation forms a mixture of diastereomers comprising both the (R) and (S) stereochemistry at the oxidized carbon. In certain embodiments, the SeO₂-mediated oxidation is stereoselective. In certain embodiments, the SeO₂-mediated oxidation forms stereoselectively an (R) stereocenter at the oxidized carbon. In certain embodiments, the SeO₂-mediated oxidation forms stereoselectively an (S) stereocenter at the oxidized carbon.

### 3-t-Butyldimethylsilyloxy-16-oxopregn-17(20)-ene (I-79)

To a solution of oxalyl chloride (0.83 ml) in dry CH₂Cl₂ (10 ml) at −78° C., was added a solution of DMSO (1.8 ml) in dry CH₂Cl₂ (10 ml) in small portions over a period of 5 min. The resulting solution was stirred at −78° C. for 15 min. Solution of I-78 (2.8 g) in dry CH₂Cl₂ (15 ml) was added in small portions to the above solution at −78° C. The reaction mixture was stirred at −78° C. for 30 min. Triethylamine (4.46 ml) was added to the reaction mixture and allowed to warm to −20° C. and stirred at −20° C. for 30 min. To the reaction mixture at −20° C. was added water (25 ml) in small portions and the resulting mixture was extracted with CH₂Cl₂ (3×20 ml). Combined organic portions were washed with water (2×15 ml), brine (20 ml), dried over sodium sulfate and concentrated to obtain crude product which was purified by silica gel (100-200 mesh) column chromatography eluting with ethyl acetate-hexane (2:98). Yield, 1.5 g (55%). TLC: ethyl acetate-hexane (20:80). Rf, 0.7. M.P.: 210-212° C.

### 3-t-Butyldimethylsilyloxy-16-oxo-24-norchol-22-ene (I-80)

A 50 ml two neck flask was charged with solution of compound I-79 (0.500 g, 1.16 mmol) in dry THF (6 ml), CuI (0.220 g, 1.16 mmol) was added. The resulting solution was degassed, cooled to 0° C. and treated with 1 M THF solution of vinyl magnesium bromide (3.48 ml) in small portions. The reaction mixture was stirred at 40° C. for 2 hr and then treated with a saturated solution of NH₄Cl (25 ml) and the aqueous portion was extracted with CH₂Cl₂ (3×15 ml). The combined dichloromethane portion was washed with water (2×15 ml), brine (20 ml), dried over sodium sulfate and concentrated to obtain crude product, which was purified by silica gel (100-200 mesh) column chromatography eluting with ethyl acetate-hexane (0.5:99.5). Yield, 271 mg (51%). TLC: Ethyl acetate-hexane (1:9). Rf, 0.7. M.P.: 132-134° C.

### Example 9

Compounds I-5, I-6, I-7, and I-8 were prepared from dehydroepiandrosterone (DHEA) according to the scheme below.

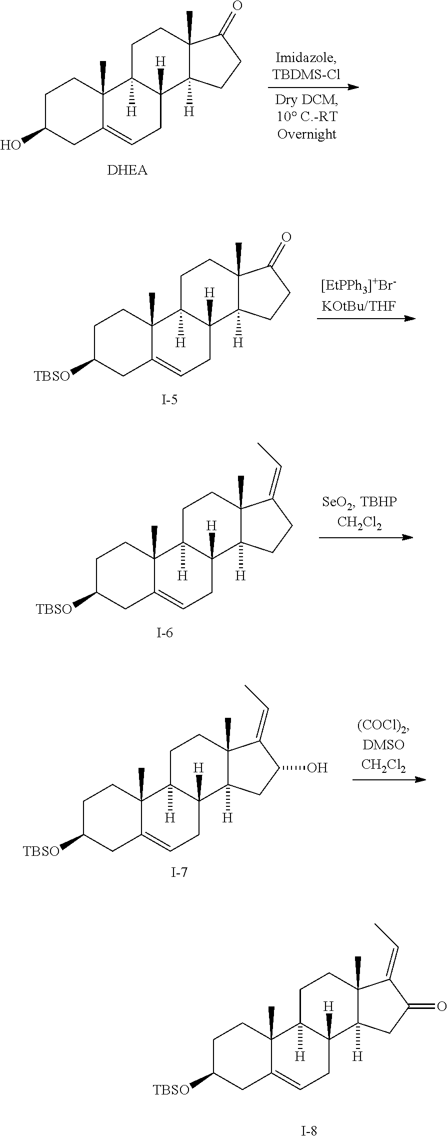

A flask was charged with DHEA (1.0 g, 3.467 mmol) and dichloromethane (10 ml) the mixture was cooled to 0° C. and imidazole (0.28 g, 4.16 mmol) was added. Reaction mixture was stirred for 10 min. Then TBDMS-Cl (0.627 g, 4.16 mmol) was added. Resulting mixture was stirred at R.T. for 12 hrs. TLC showed complete consumption of starting material. Water (20 ml) was added to the reaction mixture and extracted with DCM (3×20 ml). Combined organic layer was given water (15 ml) & brine (20 ml) wash. Dried on sodium sulphate, filtered and concentrated under reduced pressure. Crude product was purified by column chromatography using silica gel (60-120 mesh). Compound I-5 was eluted in ethyl acetate:hexane (1:99). Yield, 1.1 g (79%). TLC: Ethyl acetate-hexane (1:9). Rf, 0.4.

### Example 10

Compounds I-5, I-6, I-7, I-8, and I-21 may also be synthesized from DHEA using the following route.

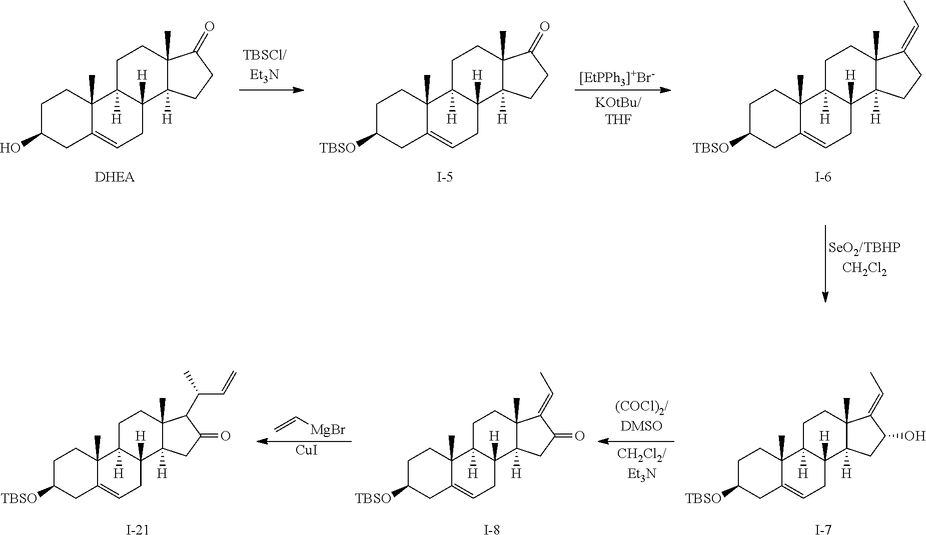

### Example 11

Compounds I-60, I-61, I-56, I-62, I-65, I-81 and I-83 were synthesized according to the following schemes.

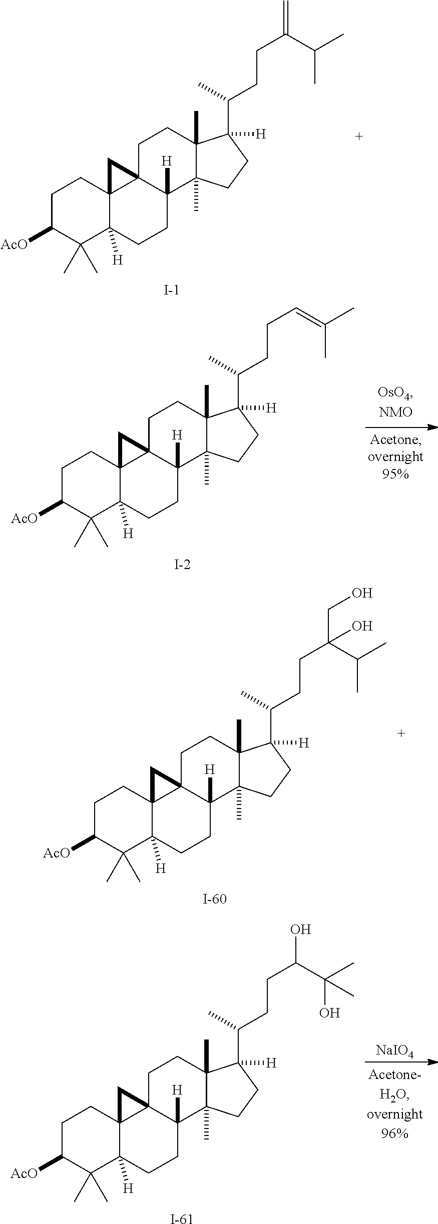

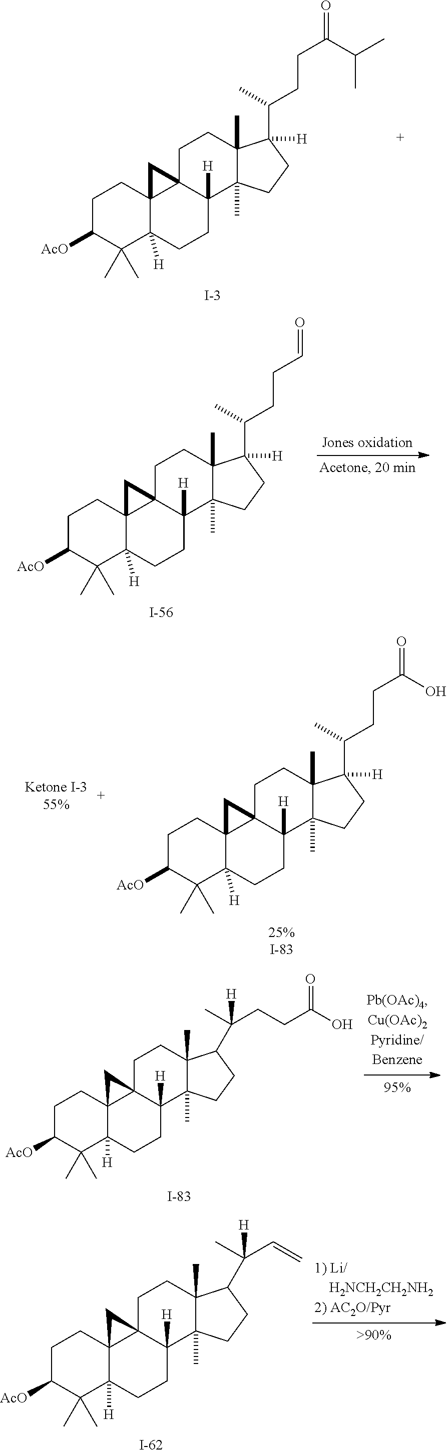

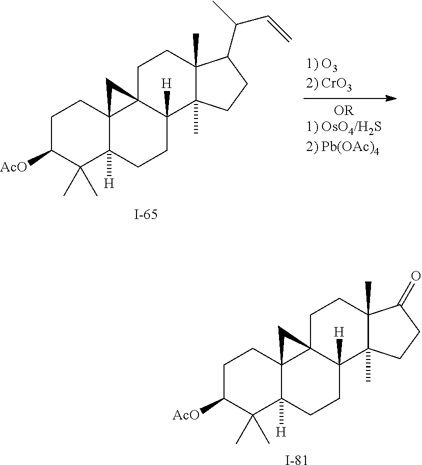

### Example 12

Compounds 1-9, I-41, I-45, I-46, and I-50 may be synthesized from compound I-8 according to the following schemes.

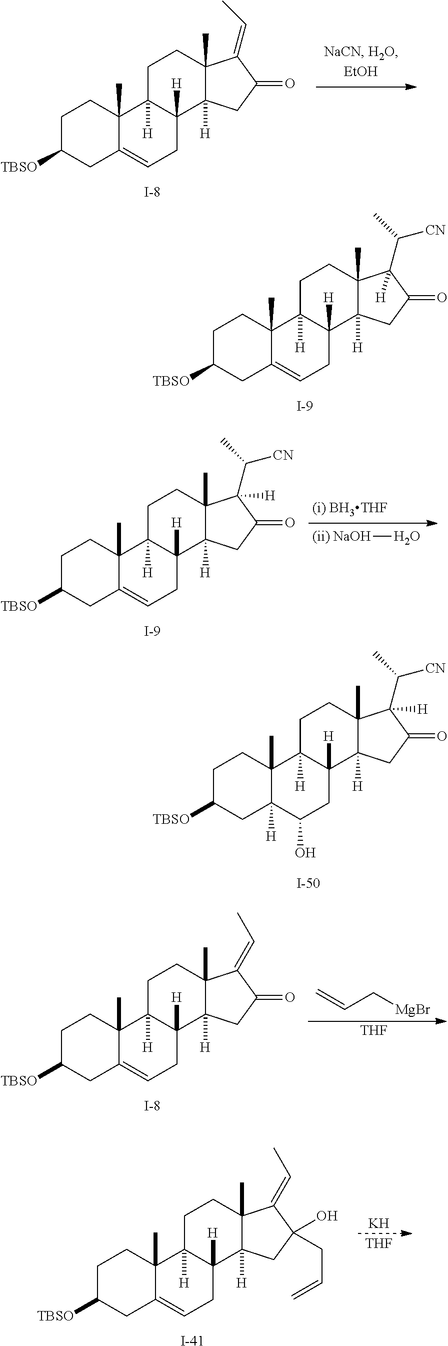

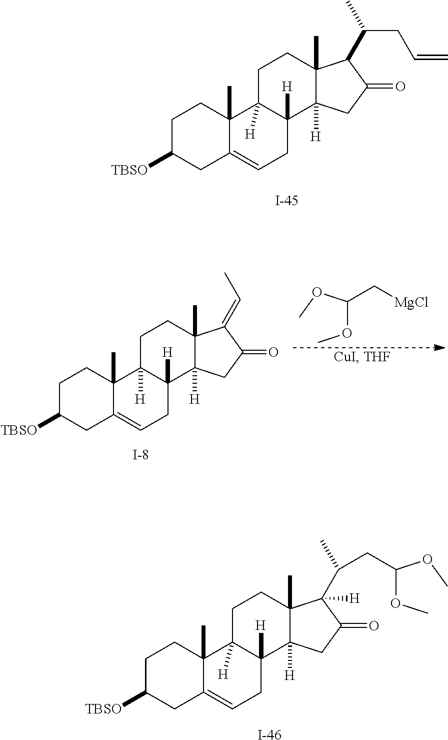

### Example 13

Compound I-47 may be synthesized from compound I-21 according to the following scheme.

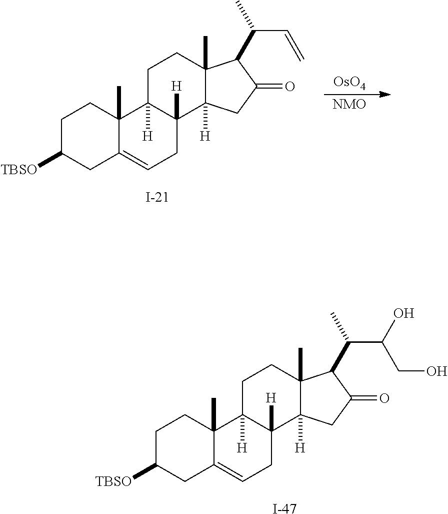

### Example 14

Compounds I-63, I-64, and I-65 may be synthesized from compound I-83 according to the following scheme.

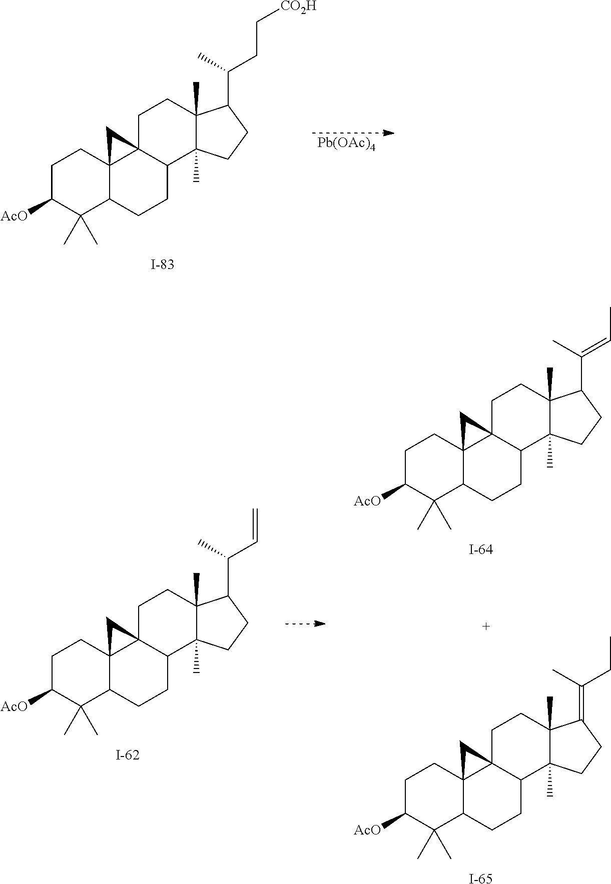

### Example 15

Other compounds of the present invention may be prepared in accordance with the schemes set forth below.

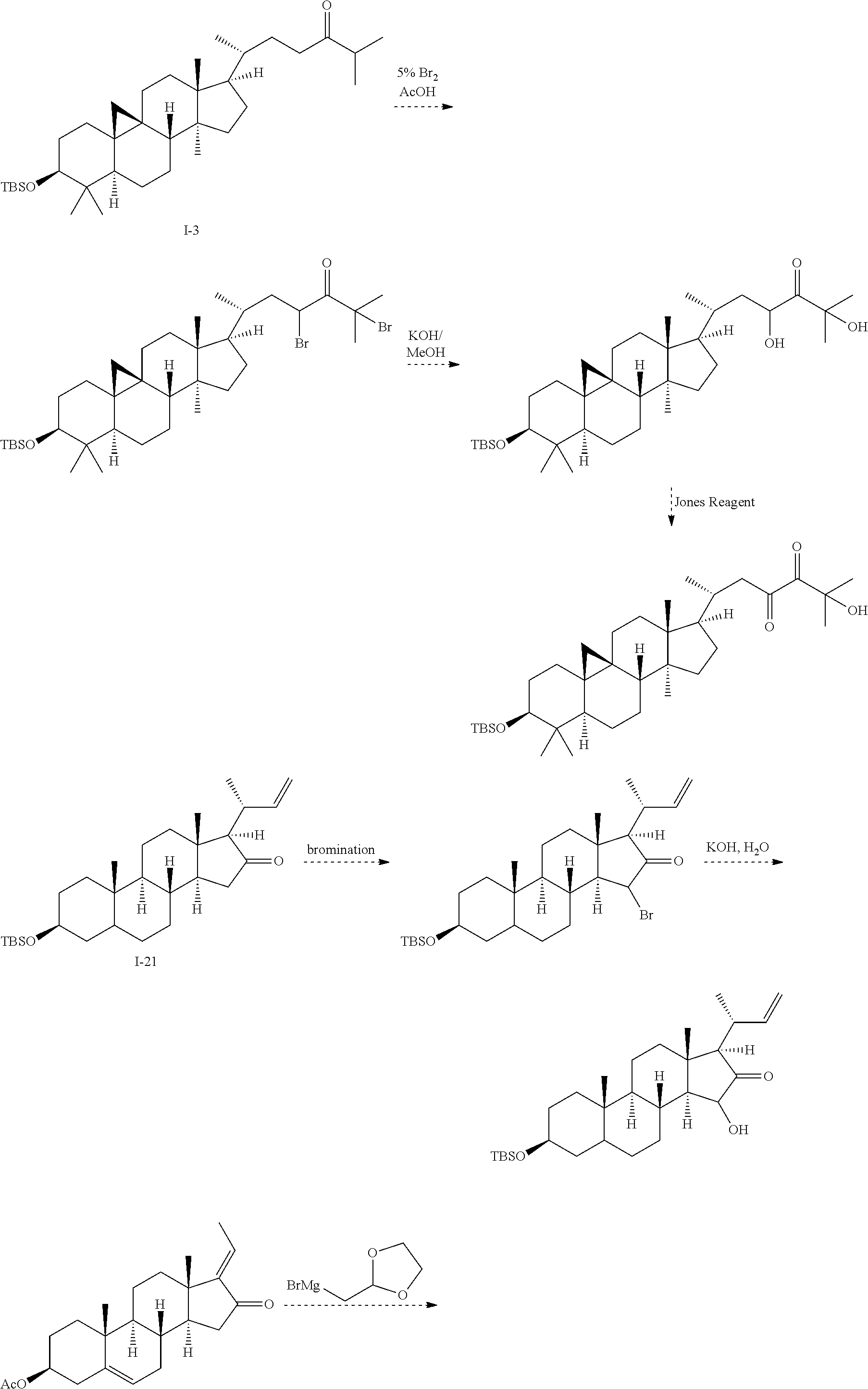

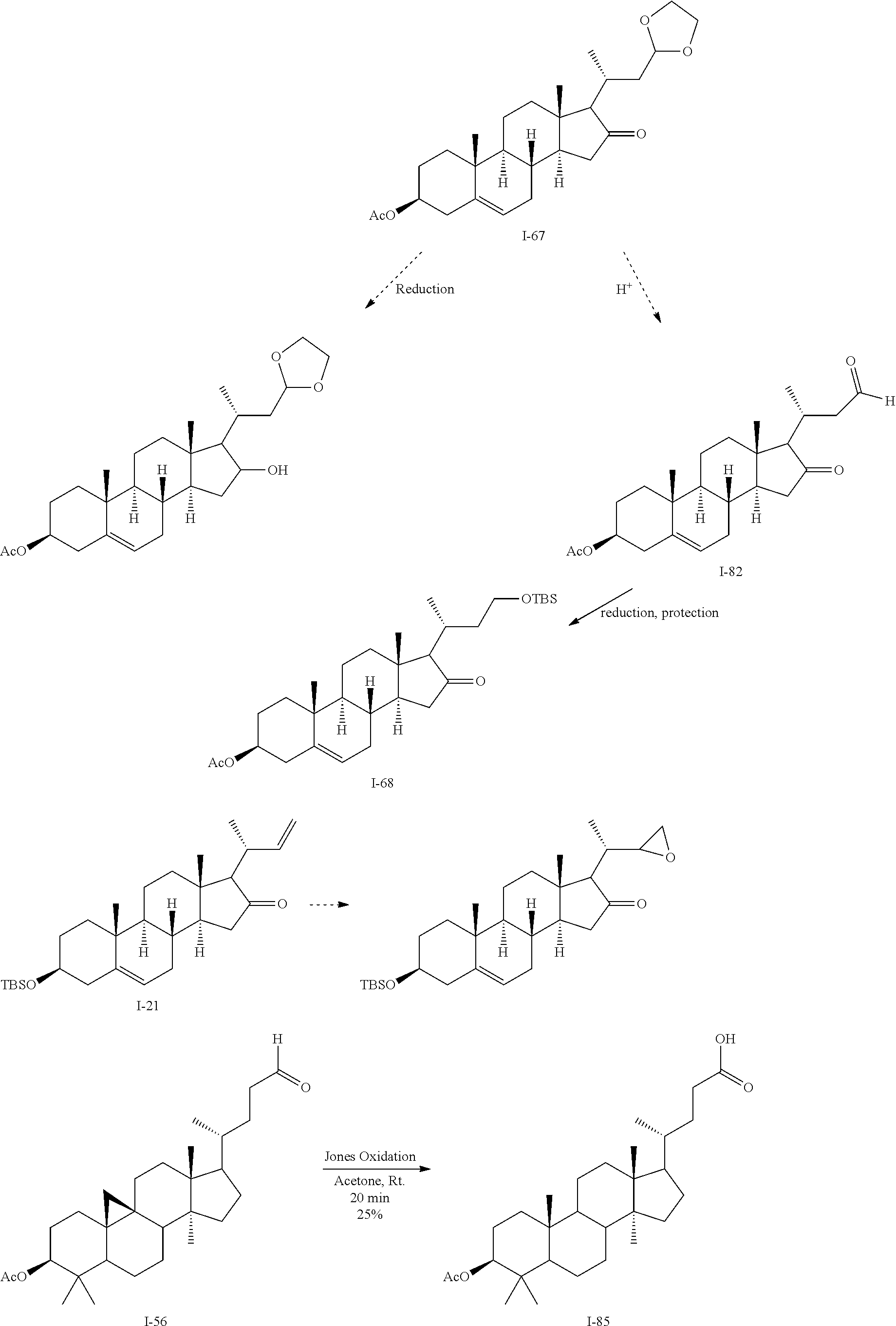

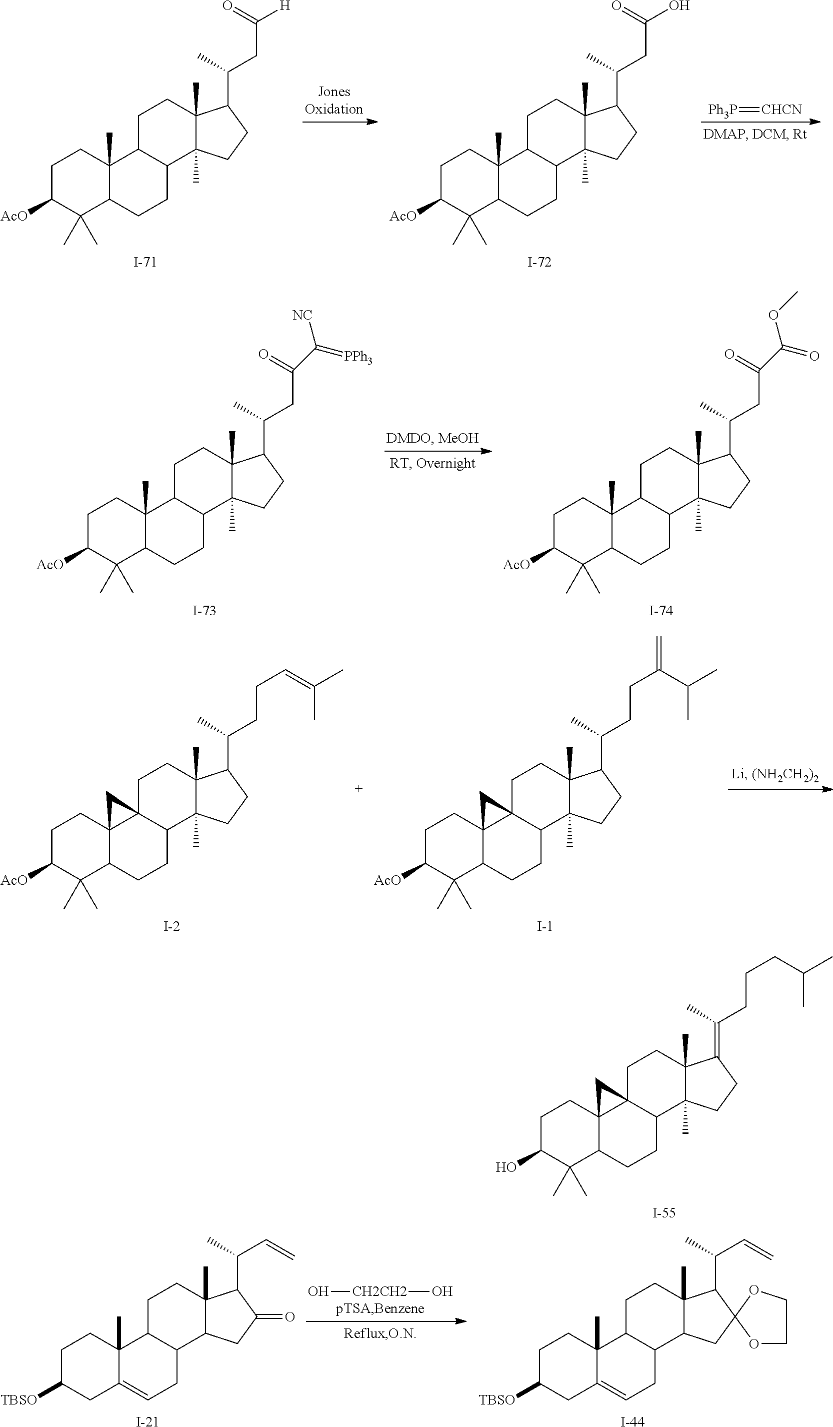

### Example 16

Compounds of the present invention may be prepared from compound A in accordance with the schemes set forth below. Compound B is prepared from compound A as described in Corey E J, et al. J Am Chem. Soc. 1994, 116, 3149-3150.

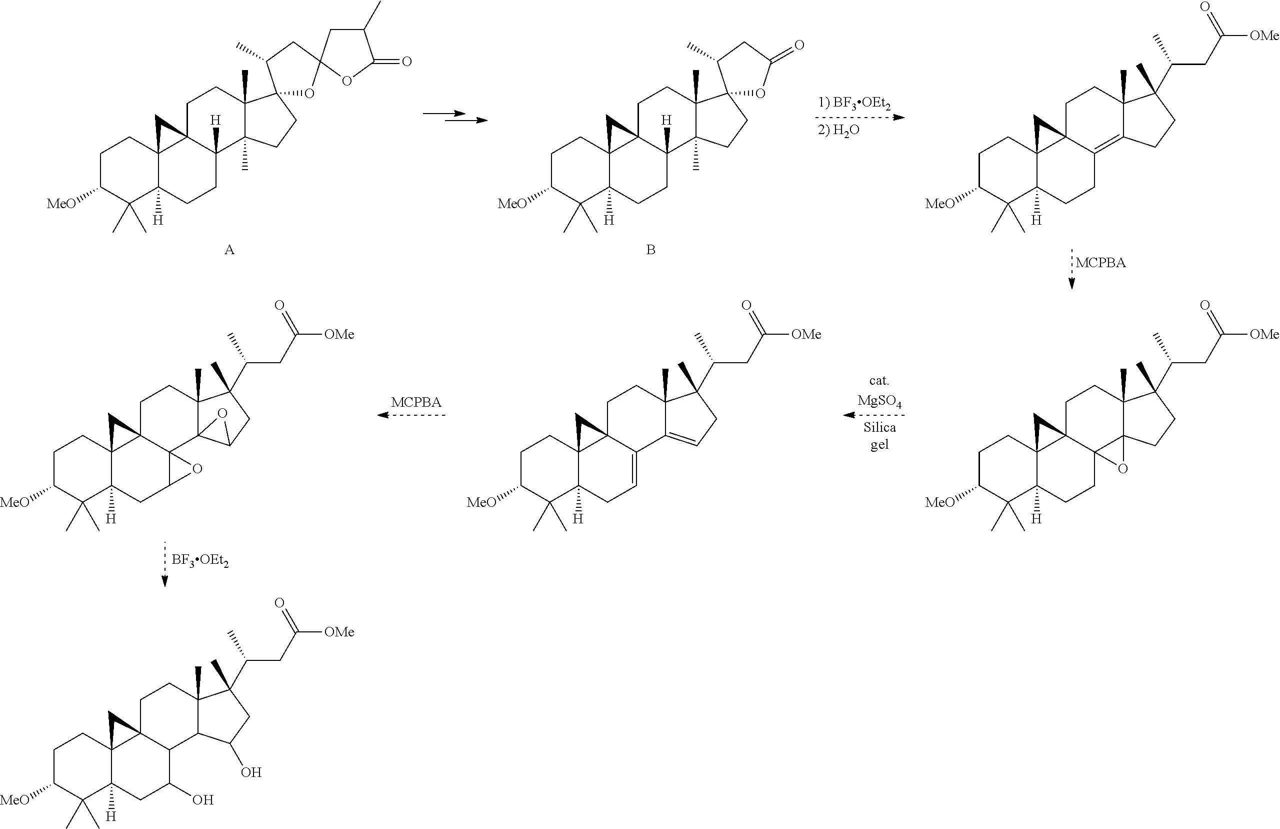

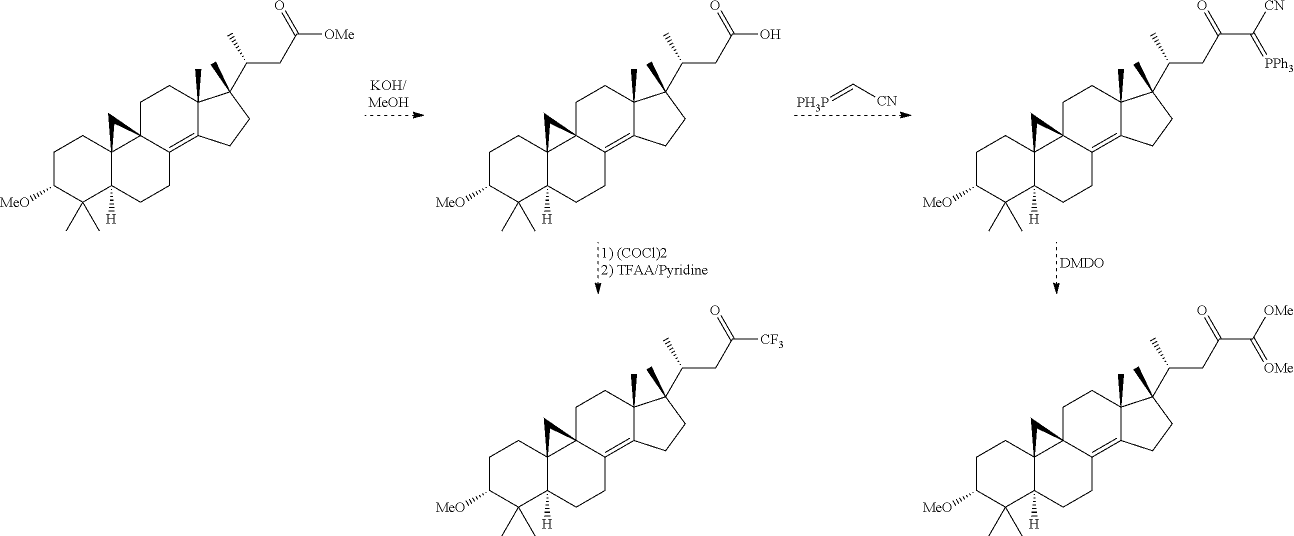

TABLE 2
Melting point and physical description data for selected compounds of formula I.

| Compound | | Melting point (° C.), |
|---|---|---|
| Number | Structure | physical description |
| I-1 + I-2 | 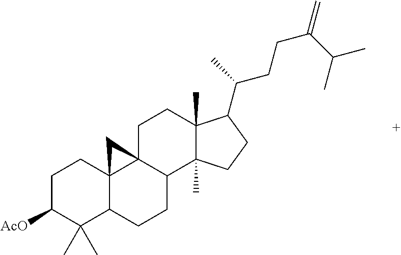 | 102-104, white solid |
| | 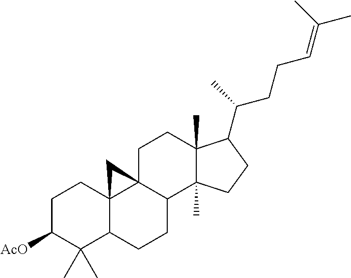 | |
| I-3 | 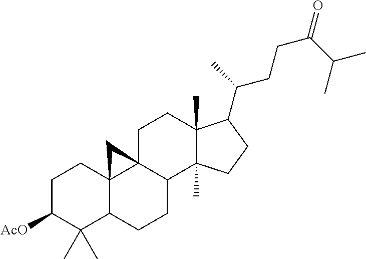 | 119-121, white solid |
| I-4 | 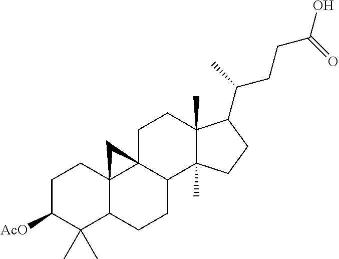 | 201-212, white solid |
| I-6 | 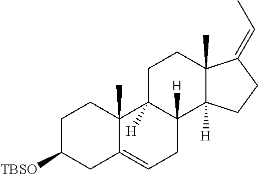 | 128-130, white solid |
| I-7 | 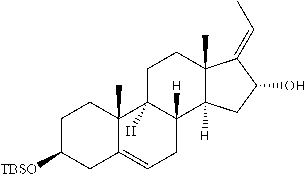 | 137-139, white solid |
| I-8 | 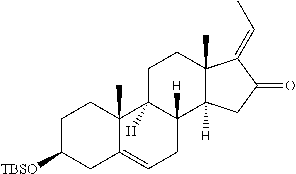 | 161-163, white solid |
| I-10 | 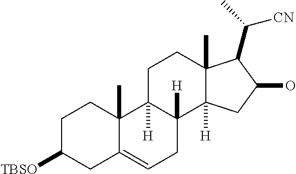 | 290-292 |
| I-13 | 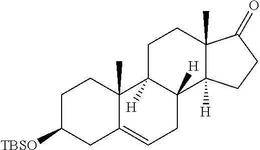 | 150-152, white solid |
| I-14 | 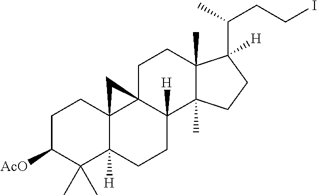 | 147-149, white solid |
| I-17 | 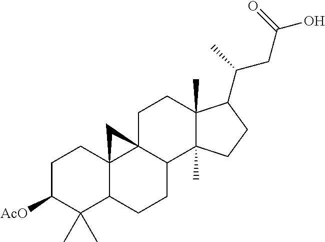 | 222-224, white solid |
| I-18 | 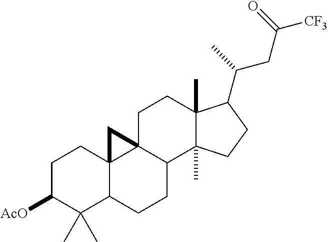 | 170-173, white solid |
| I-19 | 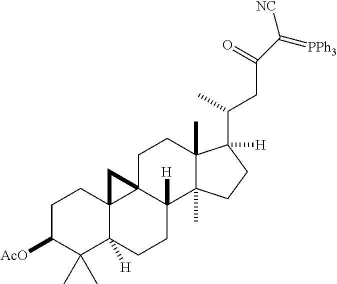 | 203-205 |
| I-20 | 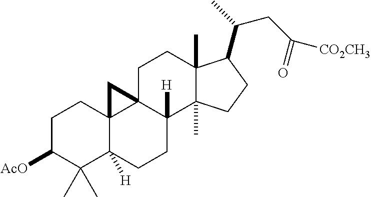 | 120-122, white solid |
| I-21 | 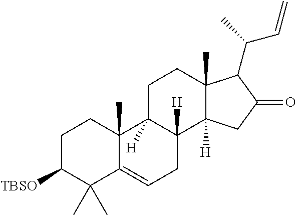 | 148-150, white solid |
| I-28 | 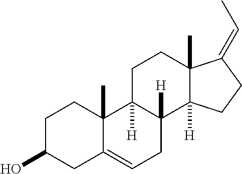 | 168-170 |
| I-29 | 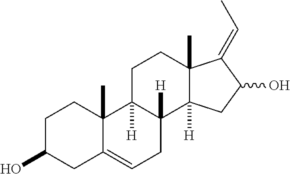 | 151-154 |
| I-30 | 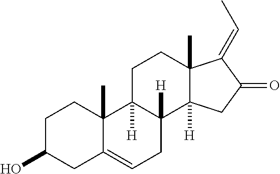 | 124-126 |
| I-31 | 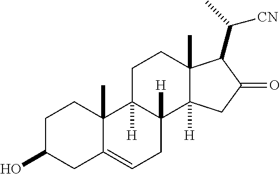 | 124-126 |
| I-40 | 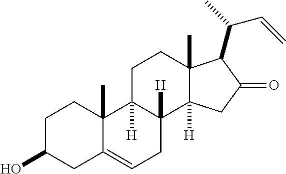 | 125-127 |
| I-41 | 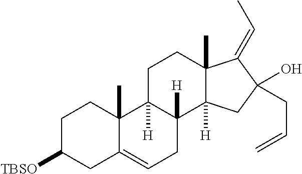 | Gum |
| I-42 | 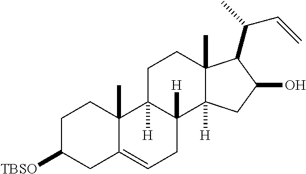 | Gum |
| I-43 | 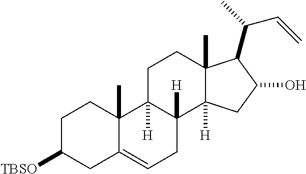 | 156-158 |
| I-44 | 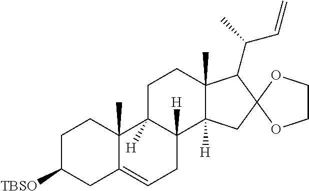 | 112-114, white solid |
| I-47 | 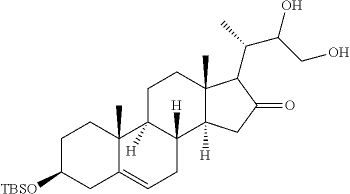 | 172-175, white solid |
| I-49 | 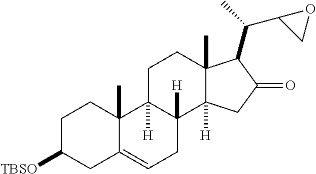 | 103-104 |
| I-51 | 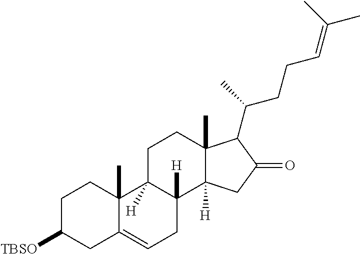 | Colorless, sticky solid |
| I-53 | 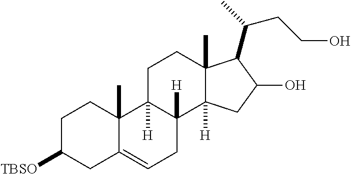 | Gum |
| I-54 | 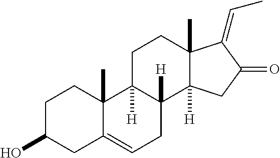 | 112-113 |
| I-58 | 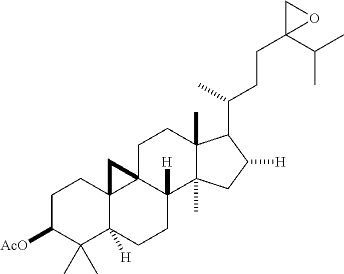 | 129-131 |
| I-59 | 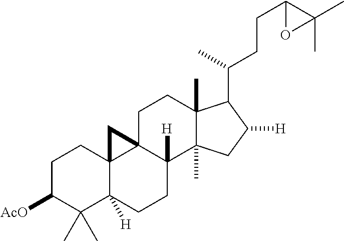 | 141-143 |
| I-62 | 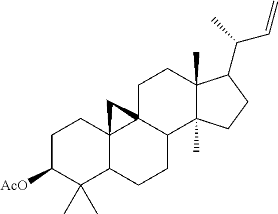 | 145-147, white solid |
| I-69 | 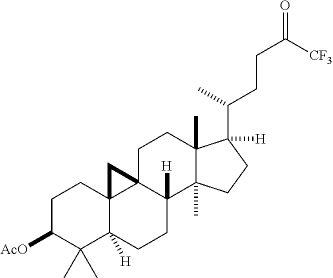 | 133-135 |
| I-70 | 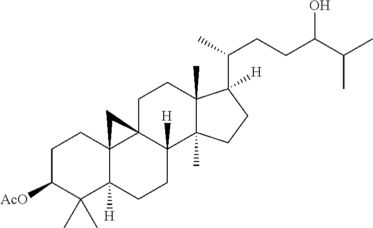 | 126-128, white solid |
| I-72 | 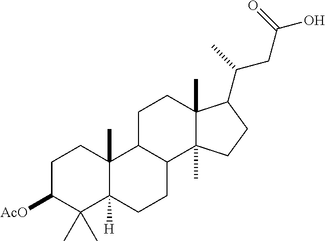 | 203-205, white solid |
| I-74 | 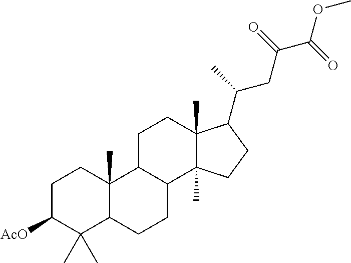 | 125-127, white solid |
| I-76 | 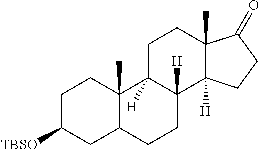 | 159-162, white solid |
| I-77 | 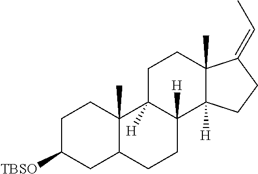 | 136-137, white solid |
| I-78 | 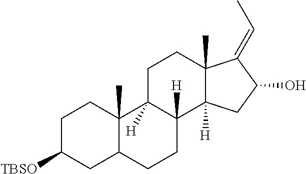 | 145-147, white solid |
| I-79 | 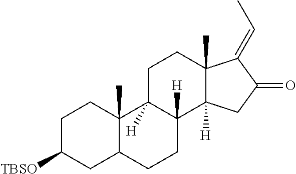 | 210-212, white solid |
| I-80 | 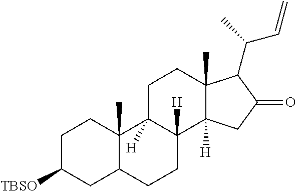 | 132-134, white solid |
| I-84 | 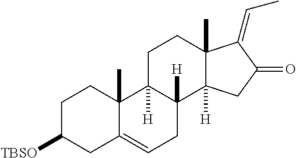 | 138-142 |
| I-86 | 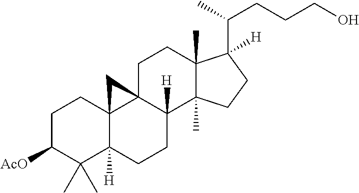 | 144-146, white solid |

### Biological Assays

### A. Assay to Determine the Ability of a Compound of Formula I to Inhibit Aβ-42

Compounds of the present invention may be assayed as inhibitors of amyloid-beta (1-42) peptide in vitro or in vivo. Such assay methods are described in detail in U.S. Pat. No. 6,649,196, the entirety of which is hereby incorporated herein by reference.

Compounds of the present invention were found to selectively lower amyloid-beta (1-42) peptide according to the cell-based assay performed in substantially the same manner as described in U.S. Pat. No. 6,649,196. In certain embodiments, compounds I-2 through I-22 were found to selectively lower amyloid-beta (1-42) peptide at 10 μM. In other embodiments, compounds I-3, I-8, I-9, I-12, and I-17 were found to selectively lower amyloid-beta (1-42) peptide at 100 nM.

### B. Assay to Determine Ability of a Compound of Formula I to Affect the Ratio of Total Aβ

Compounds of the present invention may be assayed to determine their effect on the total ration of amyloid-β(1-42) peptide in vitro using an assay protocol substantially similar to that described by Wang et al., J. Biol. Chem. 1996, 50:31894-31902, The Profile of Soluble Amyloid-β Protein in Cultured Cell Media, the entirety of which is hereby incorporated herein by reference. This assay quantifies amyloid-β protein using immunoprecipitation and mass spectrometry (IP-MS).

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

We claim:

1. A compound of formula I: 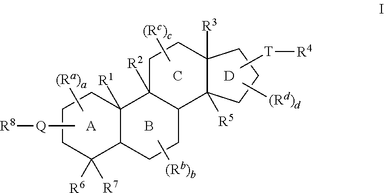
or a pharmaceutically acceptable salt thereof, wherein:
each of Ring A, Ring B, Ring C, and Ring D is independently saturated, partially unsaturated or aromatic;
R¹ and R² are each independently halogen, R, OR, a suitably protected hydroxyl group, SR, a suitably protected thiol group, N(R)₂, or a suitably protected amino group, or R¹ and R² are taken together with their intervening atoms to form a 3-7 membered saturated, partially unsaturated, or aryl ring having 0-2 heteroatoms independently selected from nitrogen, oxygen, or sulfur;
each R is independently hydrogen, an optionally substituted C₁₋₆ aliphatic group, or an optionally substituted 3-8 membered saturated, partially unsaturated, or aryl ring having 0-4 heteroatoms independently selected from nitrogen, oxygen, or sulfur, wherein:
two R on the same nitrogen atom are optionally taken together with said nitrogen atom to form a 3-8 membered saturated, partially unsaturated, or aryl ring having 1-4 heteroatoms independently selected from nitrogen, oxygen, or sulfur;
R³ and R⁵ are each independently selected from halogen, R, OR, a suitably protected hydroxyl group, SR, a suitably protected thiol group, SO₂R, OSO₂R, N(R)₂, a suitably protected amino group, NR(CO)R, NR(CO)(CO)R, NR(CO)N(R)₂, NR(CO)OR, (CO)OR, O(CO)R, (CO)N(R)₂, or O(CO)N(R)₂, wherein R³ and R^{d} optionally form an epoxide or R⁵ and R^{d} optionally form an epoxide;
R⁶ and R⁷ are each independently selected from halogen, R, OR, SR, or N(R)₂, or R⁶ and R⁷ are taken together with their intervening atoms to form a 3-7 membered saturated, partially unsaturated, or awl ring having 0-2 heteroatoms independently selected from nitrogen, oxygen, or sulfur;
T is independently a valence bond or an optionally substituted straight or branched, saturated or unsaturated, C₁₋₁₀ bivalent hydrocarbon chain wherein up to two methylene units of T are optionally and independently replaced by —O—, —N(R^{x})—, —S—, —C(O)—, —S(O)—, or —S(O)₂—, wherein two adjacent methylene units of T are optionally taken together with their intervening atoms to form an epoxide;
R⁴ is CN, C(R′)₃, C(R′)₂C(R″)₃, R, OR, a suitably protected hydroxyl group, SR, a suitably protected thiol group, SO₂R, OSO₂R, N(R)₂, a suitably protected amino group, NR(CO)R, NR(CO)(CO)R, NR(CO)N(R)₂, NR(CO)OR, (CO)OR, O(CO)R, (CO)N(R)₂, or O(CO)N(R)₂;
each of R′ and R″ is independently selected from R, OR, SR, SO₂R, OSO₂R, ═P(R)₃, N(R)₂, NR(CO)R, NR(CO)(CO)R, NR(CO)N(R)₂, NR(CO)OR, (CO)OR, O(CO)R, (CO)N(R)₂, or O(CO)N(R)₂;
each of a, b, c, and d is 0-2;
each of R^{a}, R^{b}, and R^{c} is independently halogen, CN, R, OR, a suitably protected hydroxyl group, SR, a suitably protected thiol group, SO₂R, OSO₂R, N(R)₂, a suitably protected amino group, NR(CO)R, NR(CO)(CO)R, NR(CO)N(R)₂, NR(CO)OR, (CO)OR, O(CO)R, (CO)N(R)₂, O(CO)N(R)₂, ═O, ═S, or ═NH(R), wherein:
two R^{a} groups, or an R^{a} group and R¹, on adjacent carbon atoms are optionally taken together with their intervening atoms to form an epoxide;
two R^{b} groups, or an R^{b} group and R⁵, on adjacent carbon atoms are optionally taken together with their intervening atoms to form an epoxide; or
two R^{c} groups, or an R^{c} group and either R² or R³, on adjacent carbon atoms are optionally taken together with their intervening atoms to form an epoxide;
each R^{d} is independently halogen, CN, R, OR, a suitably protected hydroxyl group, SR, a suitably protected thiol group, SO₂R, OSO₂R, N(R)₂, a suitably protected amino group, NR(CO)R, NR(CO)(CO)R, NR(CO)N(R)₂, NR(CO)OR, (CO)OR, O(CO)R, (CO)N(R)₂, O(CO)N(R)₂, ═O, ═S, or ═NH(R), wherein:
two R^{d} groups on adjacent carbon atoms of Ring D are optionally taken together with their intervening atoms to form an epoxide;
two R^{d} groups on the same carbon atom of Ring D are taken together to form an optionally substituted, straight or branched C₁₋₁₀ alkylidene group; or
two R^{d} groups on the same carbon atom of Ring D are taken together to form an optionally substituted, saturated or unsaturated, 4-7 membered ring, having 0-2 heteroatoms independently selected from oxygen, nitrogen, or sulfur, wherein said ring formed thereby is spiro fused to Ring D;
Q is a valence bond or an optionally substituted straight or branched, saturated or unsaturated, C₁₋₆ bivalent hydrocarbon chain wherein up to two methylene units of Q are optionally and independently replaced by —O—, —N(R)—, —S—, —C(O)—, —S(O)—, or —S(O)₂—; and
R⁸ is R, a suitably protected hydroxyl group, a suitably protected thiol group, a suitably protected amino group, an optionally substituted 3-8 membered saturated, partially unsaturated, or aryl monocyclic ring having 0-4 heteroatoms independently selected from nitrogen, oxygen, or sulfur, an optionally substituted 8-10 membered saturated, partially unsaturated, or aryl bicyclic ring having 0-4 heteroatoms independently selected from nitrogen, oxygen, or sulfur, a detectable moiety, a polymer residue, a peptide, or a sugar-containing or sugar-like moiety.

2. The compound according to claim 1, wherein R¹ and R² are each independently R or OR.

3. The compound according to claim 1 wherein R¹ and R² are taken together to form a 3-6 membered saturated, partially unsaturated, or aryl ring having 0-2 heteroatoms independently selected from nitrogen, oxygen, or sulfur.

4. The compound according to claim 3, wherein R¹ and R² are taken together to form a 3-6 membered saturated carbocyclic ring.

5. The compound according to claim 4, wherein said compound is of formula I-a: 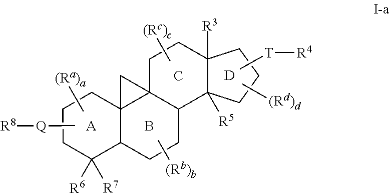
or a pharmaceutically acceptable salt thereof.

6. The compound according to claim 5, wherein said compound is of formula I-b, I-c, or I-d: 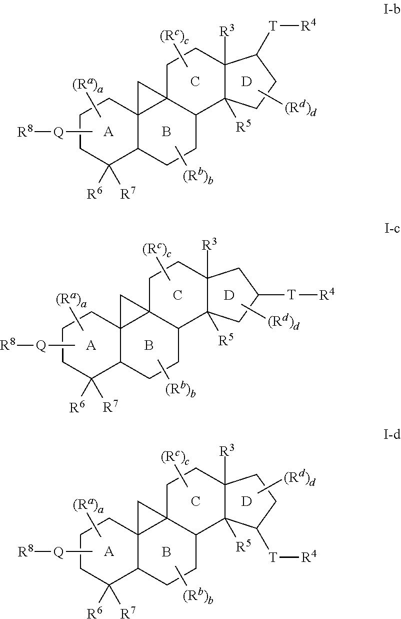
or a pharmaceutically acceptable salt thereof.

7. The compound according to any of claims 1 to 6, wherein R^{d} is R, OR, CN, ═O, or a suitably protected hydroxyl group.

8. The compound according to any of claims 1-6, wherein d is 2 and two R^{d} groups are taken together with their intervening atoms to form an epoxide to form a compound of formula I-e or I-f: 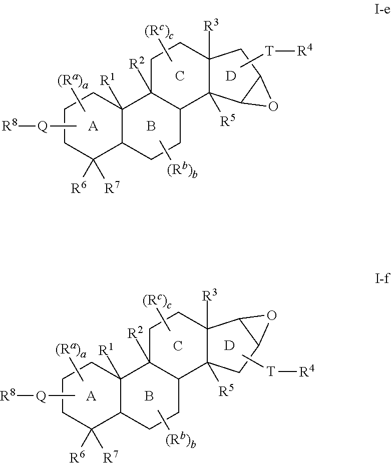
or a pharmaceutically acceptable salt thereof.

9. The compound according to any of claims 1-6, wherein d is 2 and two R^{d} groups on the same carbon atom are taken together to form an optionally substituted, straight or branched C₁₋₁₀ alkylidene group.

10. The compound according to any of claims 1 to 9, wherein T is a straight or branched C₁₋₄ bivalent hydrocarbon chain wherein one or two methylene units of T is replaced by —C(O)—, —O—, —N(R)—, or —S—.

11. The compound according to any of claims 1 to 9, wherein T is a straight or branched C₁₋₄ bivalent hydrocarbon chain, wherein two adjacent methylene units of T are taken together to form an epoxide.

12. The compound according to any of claims 1 to 10, wherein T is —CH(CH₃)CH₂CH₂C(═O)CH(CH₃)—, —CH(CH₃)CH₂CH₂CH═CH—, —CH(CH₃)CH₂CH₂—, ═CH—, —CH(CH₃)CH₂C(═O)—, or —CH(CH₃)CH═CH—.

13. The compound according to any of claims 1 to 12, wherein R⁴ is R, halogen, OR, C(O)OR, or CN.

14. The compound according to any of claims 1 to 13, wherein b is 1 and said compound is of formula I-h: 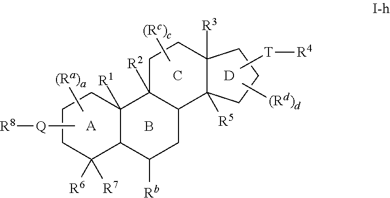
or a pharmaceutically acceptable salt thereof.

15. The compound according to claim 14, wherein R′ is ═O, R, OR, or a suitably protected hydroxyl group.

16. The compound according to any of claims 1 to 15, wherein Q is a an optionally substituted straight or branched, saturated or unsaturated, C₁₋₂ bivalent hydrocarbon chain wherein up to one methylene unit of Q is optionally replaced by —O—, —N(R)—, or —S—.

17. The compound according to any of claims 1 to 16, wherein R⁸ is a suitably protected hydroxyl group.

18. The compound according to any of claims 1 to 17, wherein said compound is of formula III, III-a, IV, or IV-a: 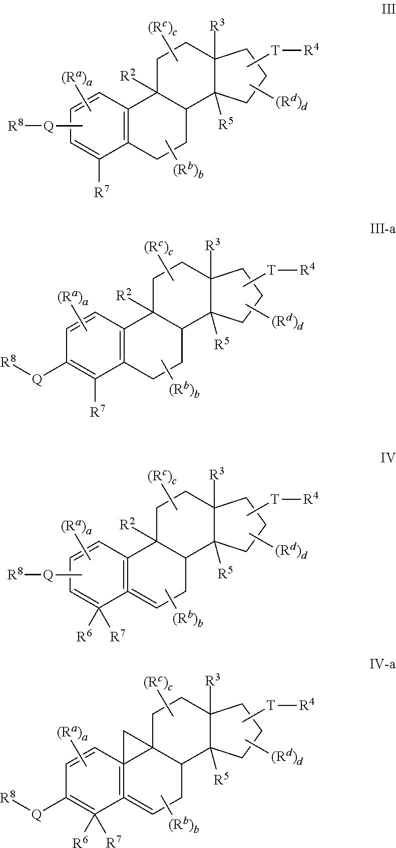
or a pharmaceutically acceptable salt thereof.

19. The compound according to any of claims 1 to 17, wherein said compound is of formula V-a, V-b, V-c, V-d, VI-a, or VI-b: 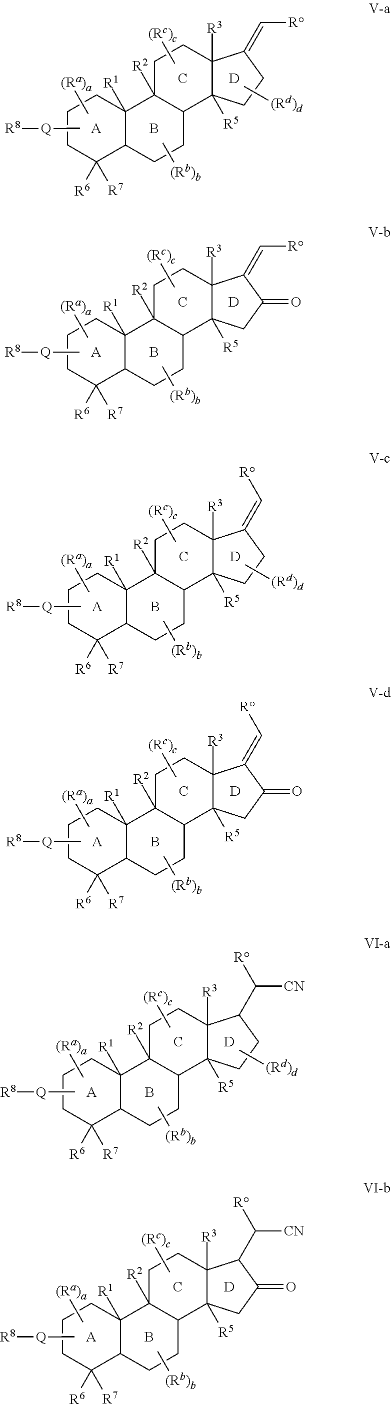
or a pharmaceutically acceptable salt thereof.

20. A compound selected from the group consisting of:

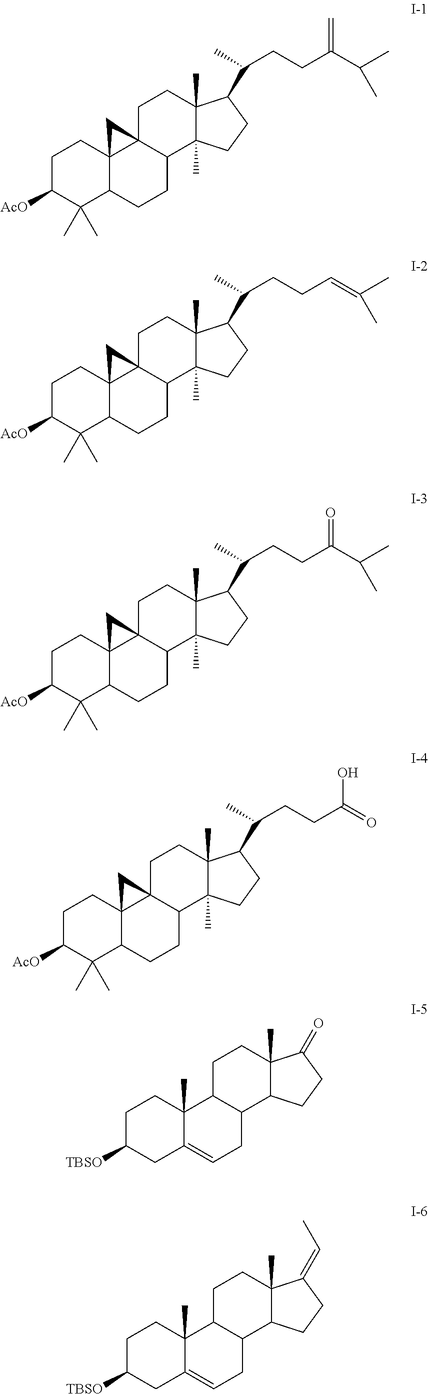

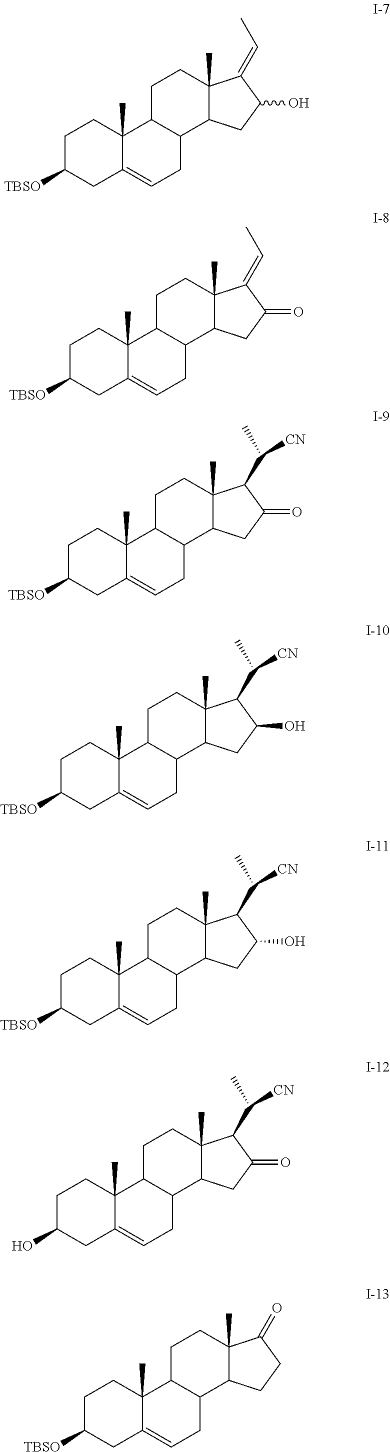

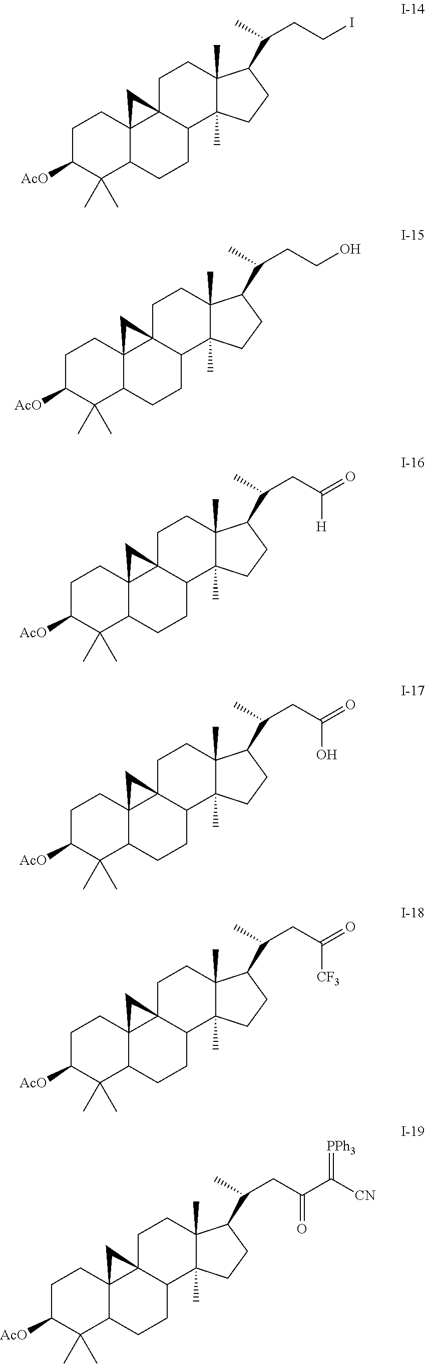

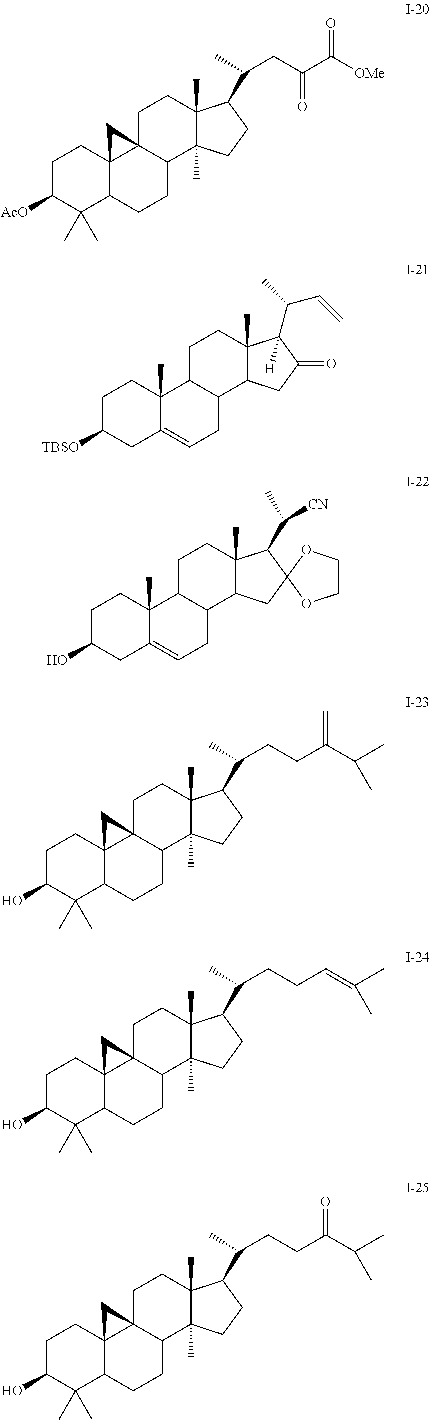

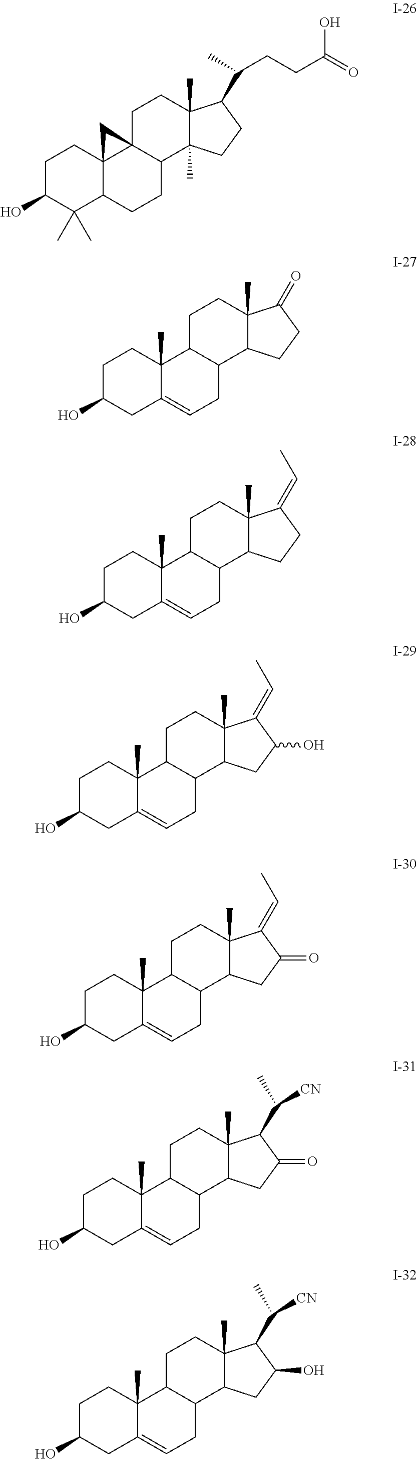

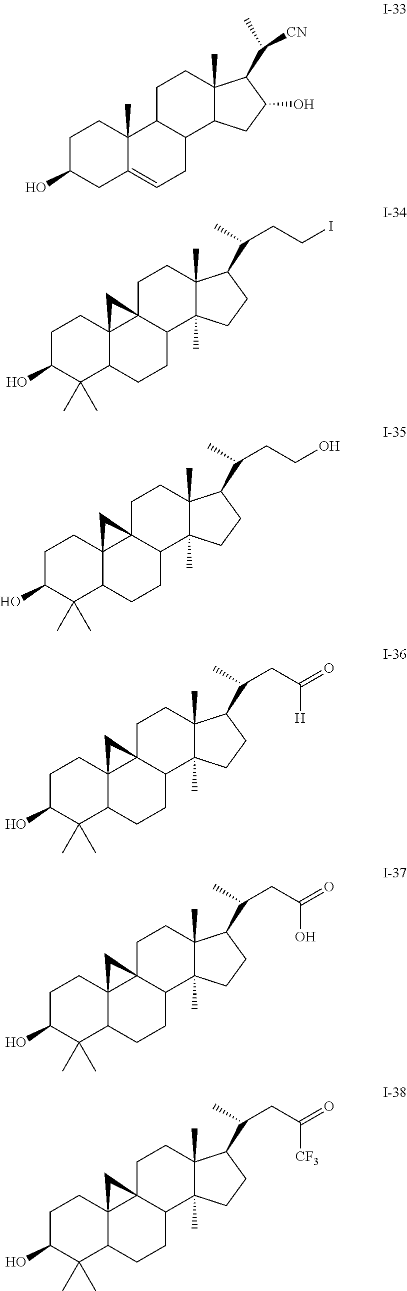

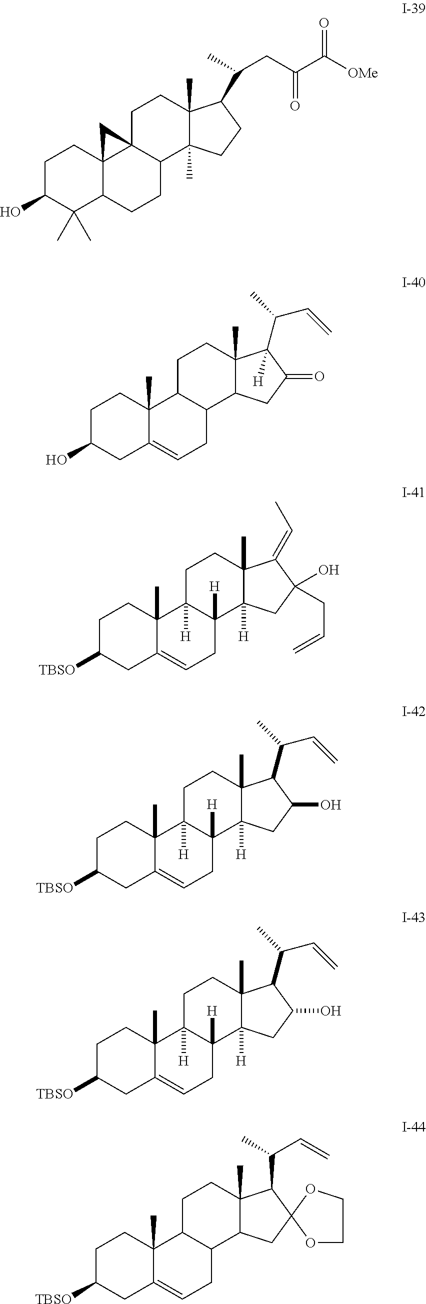

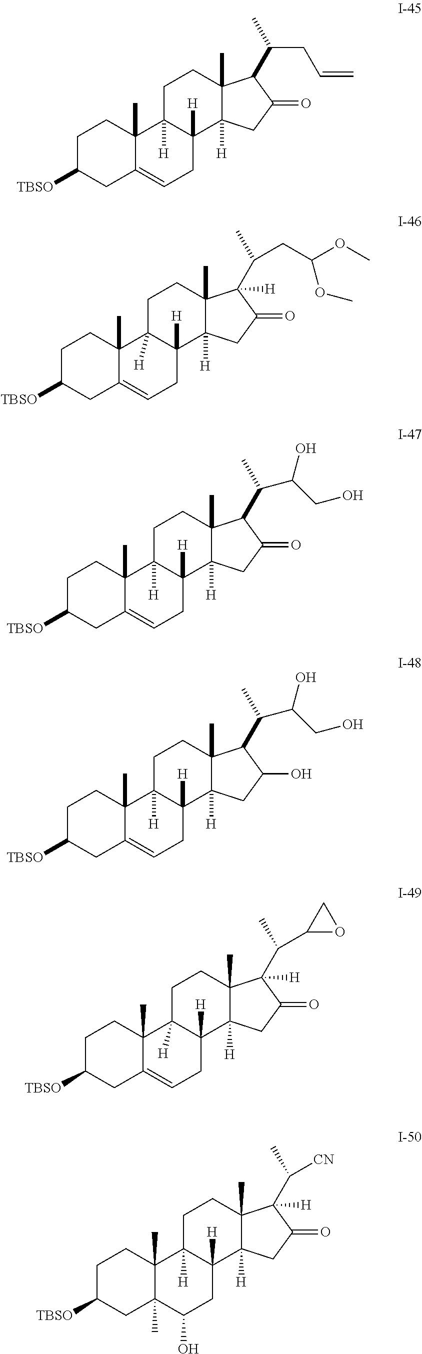

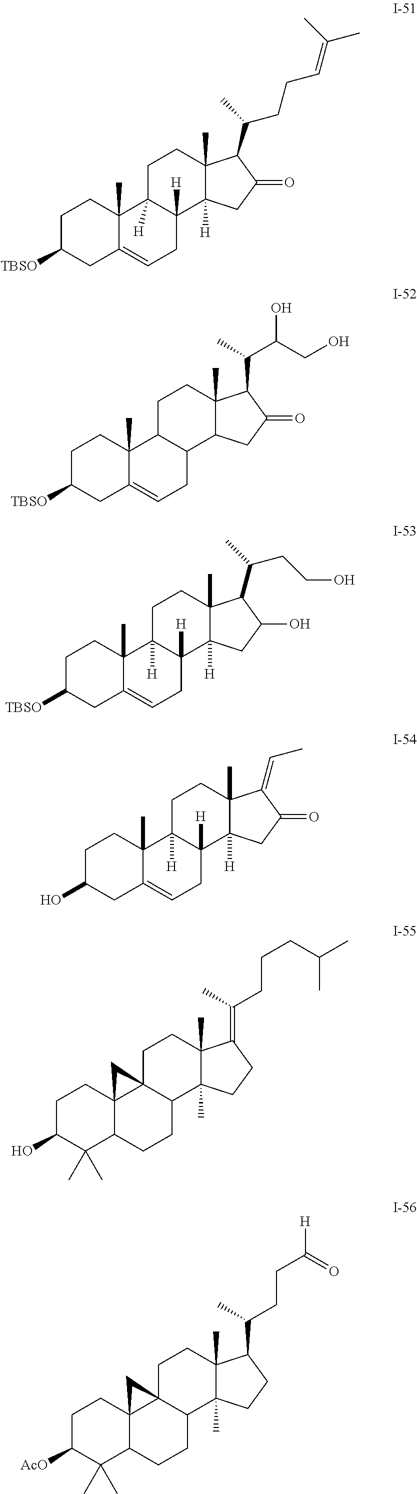

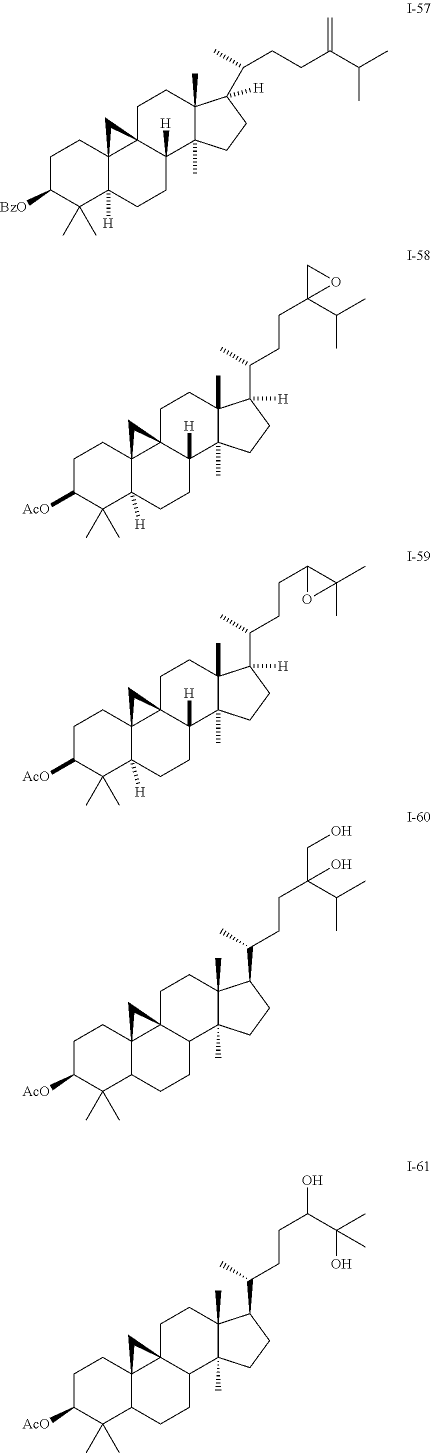

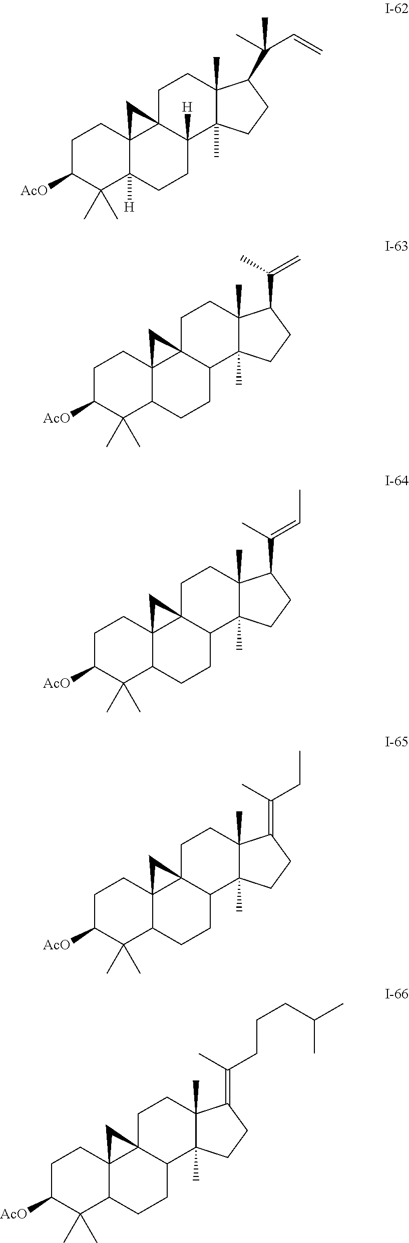

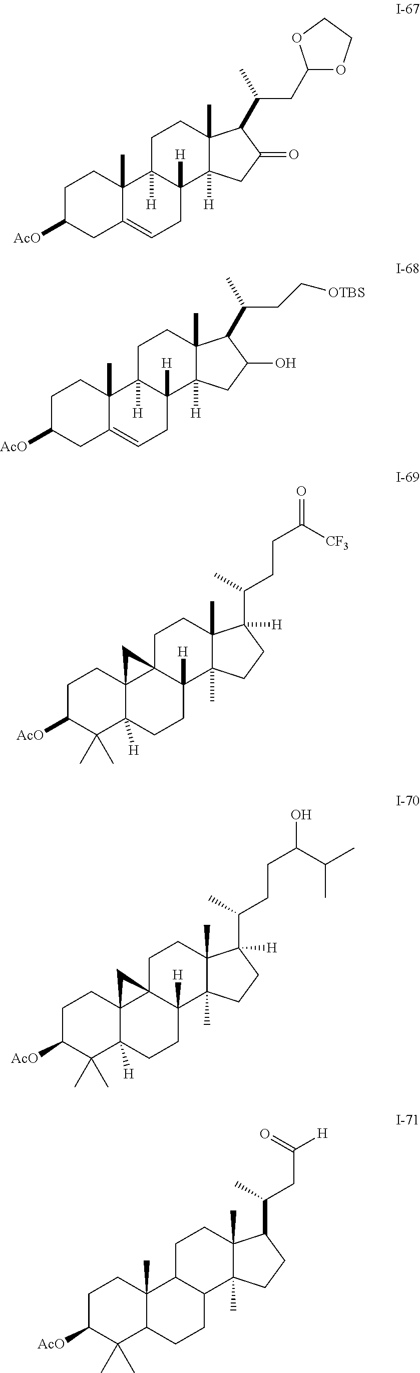

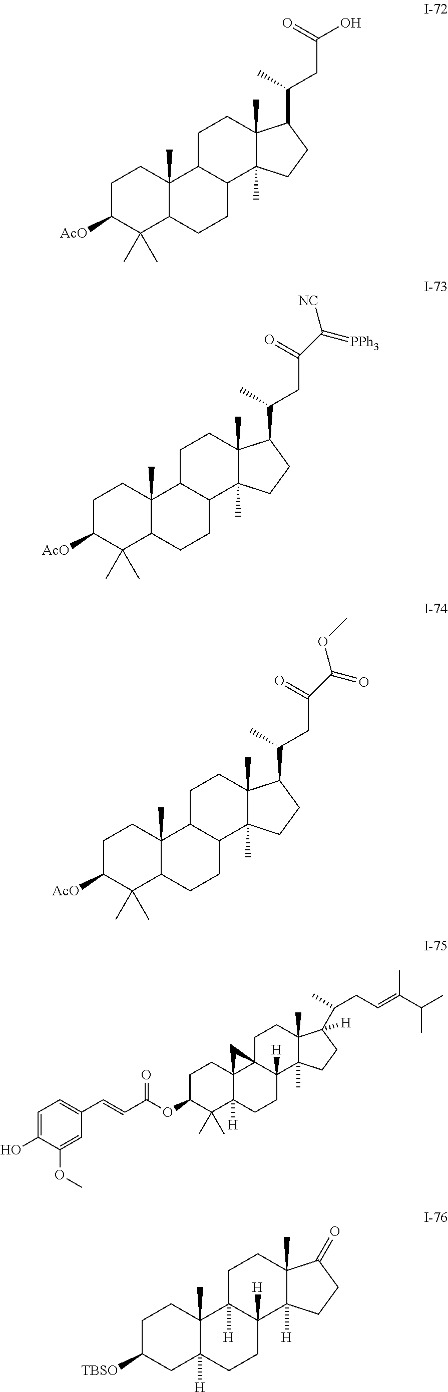

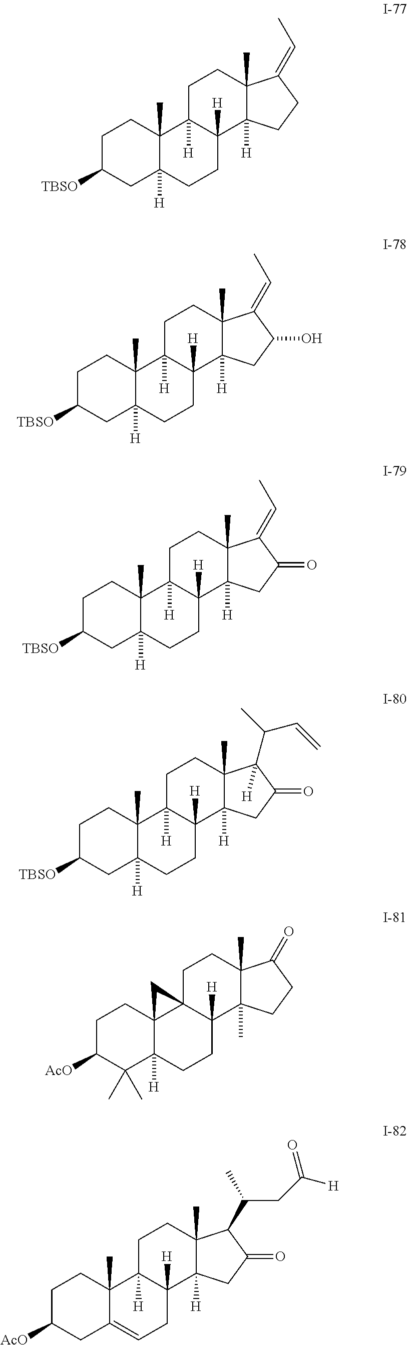

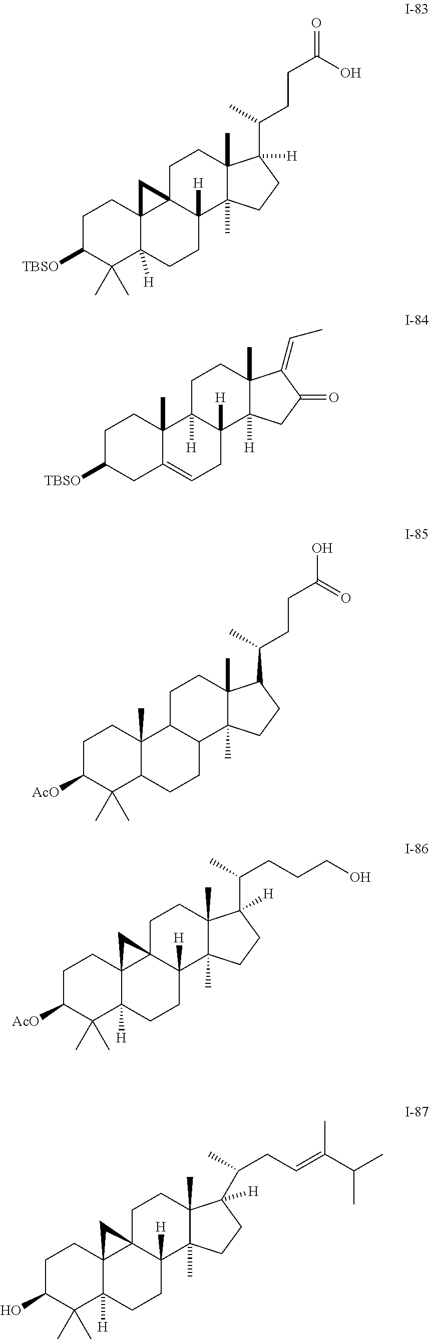

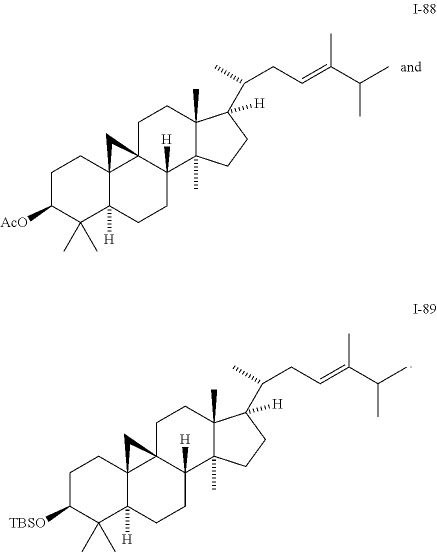

21. A composition comprising a compound according to any of claims 1 to 20, and a pharmaceutically acceptable carrier, adjuvant, or vehicle.

22. A method for inhibiting amyloid-beta peptide production in a patient, wherein said method comprises administering to said patient a composition according to claim 21.

23. The method according to claim 22, wherein said method does not affect Notch processing.

24. The method according to claim 22, wherein amyloid-beta (1-42) peptide levels are reduced and amyloid-beta (1-40) peptide levels are not substantially reduced.

25. The method according to claim 24, wherein the level of at least one of amyloid-beta (1-37) and amyloid-beta (1-39) is increased.

26. A method for treating or lessening the severity of a disorder associated with amyloid-beta (1-42) peptide, wherein said method comprises administering to a patient a composition according to claim 21.

27. The method according to claim 26, wherein said disorder is Alzheimer's disease, Parkinson's disease, Down's syndrome, inclusion body myositis, cerebral amyloid angiopathy, mild cognitive impairment, Lewy body dementia, Parkinson's disease, cataract, a Tauopathy, Huntington's disease, ALS/Lou Gerhig's disease, Type 2 diabetes, Transthyretin amyloid disease, prion disease, or CJD.

28. The method according to claim 27, wherein said disorder is Alzheimer's disease, Parkinson's disease, or Down's syndrome.

29. A method for reducing amyloid-beta (1-42) peptide levels in a patient, wherein said method comprises administering to said patient a composition according to claim 21.

30. A method for reducing amyloid-beta (1-42) peptide levels in a cell, comprising contacting said cell with a compound according to any of claims 1 to 20.
